(19) United States
(12) Patent Application Publication
(10) Pub. No.: US 2010/0111884 A1
(43) Pub. Date: May 6, 2010

(54) UV ABSORBER FORMULATIONS

(75) Inventors: Stephanie Acker, Dietwiller (FR); Sebastien Mongiat, Sierentz (FR)

Correspondence Address: Ciba Corporation;Patent Department, 540 White Plains Road, P.O. Box 2005, Tarrytown, NY, 10591, US

(73) Assignee: CIBA CORPORATION, TARRYTOWN, NY (US)

(21) Appl. No.: 12/520,894

(22) PCT Filed: Dec. 21, 2007
(86) PCT No.: PCT/EP2007/064412

§ 371 Date: Jan. 4, 2010

(30) Foreign Application Priority Data
Jan. 4, 2007 (EP) 07100108.5

(51) Int. Cl. A61K 8/49; A61Q 17/04

(52) U.S. Cl. 424/60; 424/59

(57) ABSTRACT

Disclosed is a UV filter composition comprising (a) an organic UV absorber of formula (1), wherein R₁ and R₂, independently of one another, are C₃-C₁₈alkyl; C₂-C₁₈alkenyl; a radical of the formula —CH₂—CH(OH)—CH₂—O-T₁; or R₁ and R₂ are a radical of the formula (4a); R₇ is a direct bond; a straight-chain or branched C₁-C₄alkylene radical or a radical of the formula (I) or (II); R₈, R₉ and R₁₀, independently of one another, are C₁-C₁₈alkyl; C₁-C₁₈alkoxy or a radical of the formula (III); R₁₁ is C₁-C₅ alkyl; m₁ is a number from 1 to 4; p₁ is 0; or a number from 1 to 5; A₁ is a radical of the formula (1b), (1c) or (1d); R₃ is hydrogen; C₁-C₁₀alkyl; —(CH₂CHR₅—O)n₁-T₂; or —CH₂—CH(—OH)—CH₂—O-T₂; R₄ is hydrogen; M; C₁-C₅alkyl; or —(CH₂)m₂—O-T₃; R₅ is hydrogen; or methyl; T₁, T₂ and T₃ independently from each other are hydrogen; or C₁-C₁₈alkyl; Q₁ is C₁-C₁₈alkyl, M is a metal cation; m₂ is 1 to 4; and n₁ is 1-16, (b) dimethyl capramide, (c) a cosmetically acceptable carrier; and water.

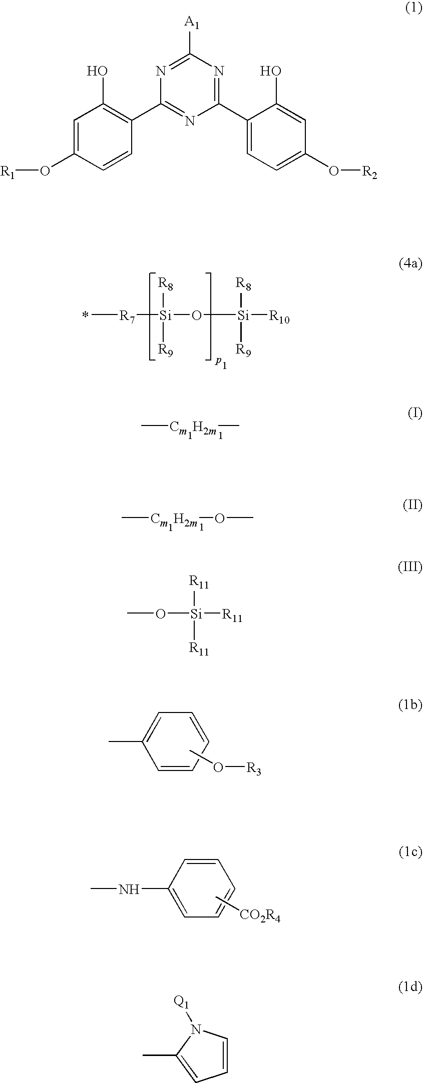

The present invention relates to improved formulations of oil-soluble organic UV absorbers.

It is well known that ultraviolet radiation (light) is harmful to human skin. Depending on the wavelength the UV radiation causes different types of skin damage. UV-B radiation (about 290 to about 320 nm) is responsible for sunburn and can cause skin cancer. UV-A radiation (about 320 to about 400 nm) while producing tanning of the skin, contributes also to sunburn and the induction of skin cancers. Moreover, the harmful effects of the UV-B radiation may be aggravated by UV-A radiation.

Therefore, an effective sunscreen formulation preferably comprises both at least one UV-A and UV-B filter and a broad band UV filter covering the full range of about 290 nm to about 400 nm to prevent the human skin from damaging by the sunlight.

A very effective organic UV braodbandfilter is Bis-Ethylhexyloxyphenol Methoxyphenyl Triazine (Tinosorb S).

Unfortunately, this UV absorber has a poor oil-solubility at a certain concentration and tends to crystallization. As a consequence the UV protection efficacy is significantly decreased.

Moreover the oil soluble UV bradband filter should be included in cosmetic sun care products without any impact on the sensorial characteristic of the emulsion. For that reason the optimal distribution of the UV absorber within the hydro-lipidic film left on the skin after spreading should be guaranteed.

It is therefore an object of the present invention to find UV absorber formulations which have an improved properties regarding the UV absorber.

Surprisingly it was found that dimethylcapramide corresponding to the formula

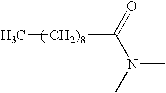

guarantees the optimal distribution of the organic UV absorber within the hydro-lipidic film of the UV absorber formulation left on the skin after spreading and promotes the highest degree of solubilisation of the organic UV absorber.

Therefore, the present invention relates to a UV absorber composition, comprising
(a) an organic UV absorber of formula

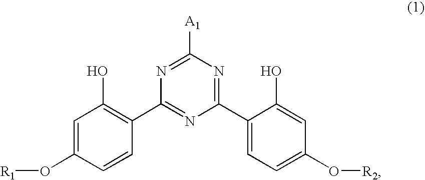

wherein
R₁ and R₂, independently of one another, are C₃-C₁₈alkyl; C₂-C₁₈alkenyl; a radical of the formula —CH₂—CH(OH)—CH₂—O-T₁; or
R₁ and R₂ are a radical of the formula (4a)

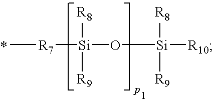

R₇ is a direct bond; a straight-chain or branched C₁-C₄alkylene radical or a radical of the formula

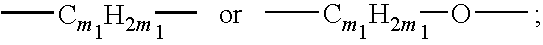

R₈, R₉ and R₁₀, independently of one another, are C₁-C₁₈alkyl; C₁-C₁₈alkoxy or a radical of the formula

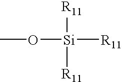

R₁₁ is C₁-C₅alkyl;
m₁ is a number from 1 to 4;
p₁ is 0; or a number from 1 to 5;
A₁ is a radical of the formula

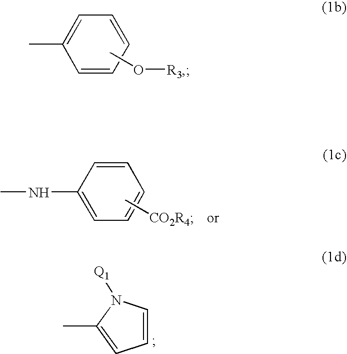

R₃ is hydrogen; C₁-C₁₀alkyl; —(CH₂CHR₅—O)ₙ_{₁}-T₂; or —CH₂—CH(—OH)—CH₂—O-T₂;
R₄ is hydrogen; M; C₁-C₅alkyl; or —(CH₂)ₘ_{₂}—O-T₃;
R₅ is hydrogen; or methyl;
T₁, T₂ and T₃ independently from each other are hydrogen; or C₁-C₈alkyl;
Q₁ is C₁-C₁₈alkyl;
M is a metal cation;
m₂ is 1 to 4; and
n₁ is 1-16;
(b) dimethyl capramide;
(c) a cosmetically acceptable carrier; and
water.

C₁-C₅alkyl, C₁-C₈alkyl, C₁-C₁₀alkyl and C₃-C₁₈alkyl are straight-chain or branched alkyl radicals such as methyl, ethyl, n-propyl, isopropyl, n-butyl, sec-butyl, tert. butyl, amyl, isoamyl tert. amyl, heptyl, octyl, isooctyl, nonyl, decyl, undecyl, dodecyl, tetradecyl, pentadecyl, hexadecyl, heptadecyl or octadecyl.

C₂-C₁₈alkenyl is, for example, allyl, methallyl, isopropenyl, 2-butenyl, 3-butenyl, isobutenyl, n-penta-2,4-dienyl, 3-methyl-but-2-enyl, n-oct-2-enyl, n-dodec-2-enyl, iso-dodecenyl, n-dodec-2-enyl or n-octadec-4-enyl.

Preferred bisresorcinyl compounds of the formula (1) are those in which A₁ is a radical of the formula

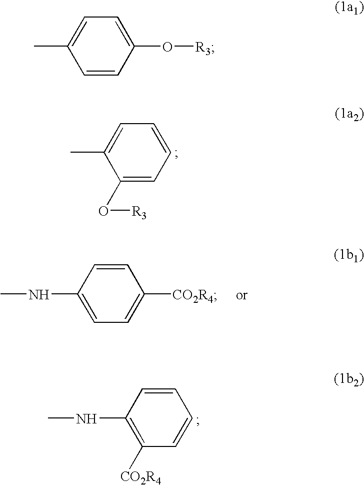

wherein
R₃ and R₄ are as defined in formulae (1a) and (1b).

Important bisresorcinyl compounds according to the invention correspond to the formula

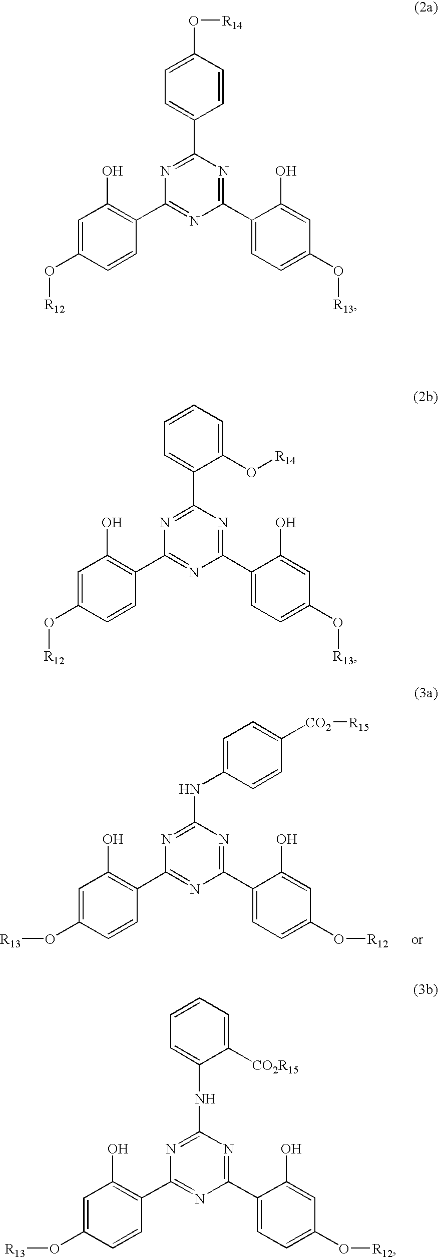

in which
R₁₂ and R₁₃, independently of one another, are C₃-C₁₈alkyl; or —CH₂—CH(—OH)—CH₂—O-T₁;
R₁₄ is C₁-C₁₀alkyl; or a radical of the formula

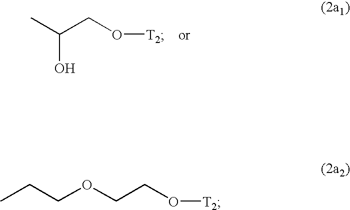

R₁₅ is hydrogen; M; C₁-C₅alkyl; or a radical of the formula —(CH₂)ₘ—O-T₂;
M is an alkali metal cation;
T₁ and T₂, independently of one another, are hydrogen; or C₁-C₅alkyl; and
m is 1 to 4.

Uppermost of interest are compounds of the formulae (2a) and (2b), wherein

R₁₂ and R₁₃, independently of one another, are C₃-C₁₈alkyl or —CH₂—CH(—OH)—CH₂—O-T₁;
R₁₄ is C₁-C₁₀alkyl; and
T₁ is hydrogen; or C₁-C₅alkyl;
and compounds of the formulae (3a) and (3b), wherein
R₁₂ and R₁₃, independently of one another, are C₃-C₁₈alkyl or —CH₂—CH(—OH)—CH₂—O-T₁;
R₁₅ is hydrogen; M; C₁-C₅alkyl; and
T₁ is hydrogen; or C₁-C₅alkyl.

Very particularly preferred are triazine compounds of the formula (2) or (3), in which R₁₂ and R₁₃ have the same meaning.

Examples of compounds of the formula (1) which may be mentioned are:
2,4-Bis-{[4-(2-ethyl-hexyloxy)-2-hydroxy]-phenyl}-6-(4-methoxyphenyl)-1,3,5-triazine;
2-(4′-methoxyphenyl)-4,6-bis(2′-hydroxy-4′-n-octyloxyphenyl)-1,3,5-triazine;
2,4-bis{[4-(3-(2-propyloxy)-2-hydroxypropyloxy)-2-hydroxy]phenyl}-6-(4-methoxyphenyl)-1,3,5-triazine;
2,4-bis{[4-(2-ethylhexyloxy)-2-hydroxy]phenyl}-6-[4-(2-methoxyethylcarboxyl)phenylamino]-1,3,5-triazine;
2,4-bis{[4-(tris(trimethylsiloxysilylpropyloxy)-2-hydroxy]phenyl}-6-(4-methoxyphenyl)-1,3,5-triazine;
2,4-bis{[4-(2″methylpropenyloxy)-2-hydroxy]phenyl}-6-(4-methoxyphenyl)-1,3,5-triazine;
2,4-bis{[4-(1′,1′,1′,3′,5′,5′,5′-heptamethyltrisilyl-2″-methylpropyloxy)-2-hydroxy]phenyl}-6-(4-methoxyphenyl)-1,3,5-triazine;
2,4-bis{[4-(3-(2-propyloxy)-2-hydroxypropyloxy)-2-hydroxy]phenyl}-6-[4-ethylcarboxyl)-phenylamino]-1,3,5-triazine; or
2,4-bis{[4-(2-ethylhexyloxy)-2-hydroxy]phenyl}-6-(1-methylpyrrol-2-yl)-1,3,5-triazine.

The bis(resorcinyl)triazines as used in the present invention can be prepared by manners known per se, as described for example in EP 775698 B1.

The UV absorber composition according to the present invention may comprise one or more than one additional UV absorbers (component (d)) as described in the Tables 1 and 2.

Preference is given to the use of mixing ratios of the compound of formula (1) according to the present invention and optionally further UV absorbers as described in the Table 1 and 2 from 1:99 to 99:1, preferably from 1:95 to 95:1 and most preferably from 10:90 to 90:10, based on weight.

Of special interest are mixing ratios of from 20:80 to 80:20 and most preferably from 40:60 to 60:40.

TABLE 1
Suitable UV filter substances which can be additionally used with the UV absorbers
according to the present invention

p-aminobenzoic acid derivatives, for example 4-dimethylaminobenzoic acid 2-ethylhexyl ester;
salicylic acid derivatives, for example salicylic acid 2-ethylhexyl ester;
benzophenone derivatives, for example 2-hydroxy-4-methoxybenzophenone and its
5-sulfonic acid derivative;
diphenylacrylates, for example 2-ethylhexyl 2-cyano-3,3-diphenylacrylate, and 3-(benzo-
furanyl) 2-cyanoacrylate;
3-imidazol-4-ylacrylic acid and esters;
benzofuran derivatives, especially 2-(p-aminophenyl)benzofuran derivatives, described in
EP-A-582 189, U.S. Pat. No. 5,338,539, U.S. Pat. No. 5,518,713 and EP-A-613 893;
polymeric UV absorbers, for example the benzylidene malonate derivatives described in
EP-A-709 080;
cinnamic acid derivatives, for example the 4-methoxycinnamic acid 2-ethylhexyl ester and
isoamyl ester or cinnamic acid derivatives described in U.S. Pat. No. 5,601,811 and WO 97/00851;
camphor derivatives, for example 3-(4′-methyl)benzylidene-bornan-2-one, 3-benzylidene-
bornan-2-one, N-[2(and 4)-2-oxyborn-3-ylidene-methyl)-benzyl]acrylamide polymer, 3-(4′-
trimethylammonium)-benzylidene-bornan-2-one methyl sulfate, 3,3′-(1,4-phenylene-
dimethine)-bis(7,7-dimethyl-2-oxo-bicyclo[2.2.1]heptane-1-methanesulfonic acid) and salts,
3-(4′-sulfo)benzylidene-bornan-2-one and salts; camphorbenzalkonium methosulfate;
hydroxyphenyltriazine compounds, for example 2-(4′-methoxyphenyl)-4,6-bis(2′-hydroxy-4′-n-
octyloxyphenyl)-1,3,5-triazine; 2,4-bis{[4-(3-(2-propyloxy)-2-hydroxy-propyloxy)-2-hydroxy]-
phenyl}-6-(4-methoxyphenyl)-1,3,5-triazine; 2,4-bis{[4-(2-ethyl-hexyloxy)-2-hydroxy]-phenyl}-
6-[4-(2-methoxyethyl-carboxyl)-phenylamino]-1,3,5-triazine; 2,4-bis{[4-(tris(trimethylsilyloxy-
silylpropyloxy)-2-hydroxy]-phenyl}-6-(4-methoxyphenyl)-1,3,5-triazine; 2,4-bis{[4-(2″-
methylpropenyloxy)-2-hydroxy]-phenyl}-6-(4-methoxyphenyl)-1,3,5-triazine; 2,4-bis{[4-
(1′,1′,1′,3′,5′,5′,5′-heptamethyltrisilyl-2″-methyl-propyloxy)-2-hydroxy]-phenyl}-6-(4-
methoxyphenyl)-1,3,5-triazine; 2,4-bis{[4-(3-(2-propyloxy)-2-hydroxy-propyloxy)-2-hydroxy]-
phenyl}-6-[4-ethylcarboxy)-phenylamino]-1,3,5-triazine;
physical sunscreens, coated or not coated, such as titanium dioxide, zinc oxide, iron oxides,
mica, MnO, Fe₂O₃, Ce₂O₃, Al₂O₃, ZrO₂ (surface coatings: polymethylmethacrylate, methicone
(methylhydrogenpolysiloxane as described in CAS 9004-73-3), dimethicone, isopropyl titanium
triisostearate (as described in CAS 61417-49-0), metal soaps such as magnesium
stearate (as described in CAS 4086-70-8), perfluoroalcohol phosphate such as C₉-C₁₅
fluoroalcohol phosphate (as described in CAS 74499-44-8; JP 5-86984; JP 4-330007)). The
primary particle size is, on average, 15 nm-35 nm and the particle size distribution is in the
range 100 nm-300 nm.
aminohydroxy-benzophenone derivatives disclosed in DE 100 11 317, EP 1 133 980 and
EP 1 046 391
phenyl-benzimidazole derivatives as disclosed in EP 1 167 358

TABLE 2
Suitable UV filter substances and adjuvants which can be additionally used with the
UV absorbers according to the present invention

| No. | Chemical Name | CAS No. |
|---|---|---|
| 1 | (+/−)-1,7,7-trimethyl-3-[(4-methylphenyl)methylene]bicyclo- | 36861-47-9 |
| | [2.2.1]heptan-2-one; p-methyl benzylidene camphor | |
| 2 | 1,7,7-trimethyl-3-(phenylmethylene)bicyclo[2.2.1]heptan-2-one; ben- | 15087-24-8 |
| | zylidene camphor | |
| 3 | (2-Hydroxy-4-methoxyphenyl)(4-methylphenyl)methanone | 1641-17-4 |
| 4 | 2,4-dihydroxybenzophenone | 131-56-6 |
| 5 | 2,2′,4,4′-tetrahydroxybenzophenone | 131-55-5 |
| 6 | 2-Hydroxy-4-methoxy benzophenone; | 131-57-7 |
| 7 | 2-Hydroxy-4-methoxy benzophenone-5-sulfonic acid | 4065-45-6 |
| 8 | 2,2′-dihydroxy-4,4′-dimethoxybenzophenone | 131-54-4 |
| 9 | 2,2′-Dihydroxy-4-methoxybenzophenone | 131-53-3 |
| 10 | Alpha-(2-oxoborn-3-ylidene)toluene-4-sulphonic acid and its salts; | 56039-58-8 |
| | Mexoryl SL | |
| 11 | 1-[4-(1,1-dimethylethyl)phenyl]-3-(4-methoxyphenyl)propane-1,3- | 70356-09-1 |
| | dione; avobenzone | |
| 12 | Methyl N,N,N-trimethyl-4-[(4,7,7-trimethyl-3-oxobicyclo[2,2,1]hept-2- | 52793-97-2 |
| | ylidene)methyl]anilinium sulphate; Mexoryl SO | |
| 22 | 3,3,5-Trimethyl cyclohexyl-2-hydroxy benzoate; homosalate | 118-56-9 |
| 23 | Isopentyl p-methoxycinnamate; isoamyl methoxy cinnamate | 71617-10-2 |
| 27 | Menthyl-o-aminobenzoate | 134-09-8 |
| 28 | Menthyl salicylate | 89-46-3 |
| 29 | 2-Ethylhexyl 2-cyano,3,3-diphenylacrylate; octocrylene | 6197-30-4 |
| 30 | 2-ethylhexyl 4-(dimethylamino)benzoate | 21245-02-3 |
| 31 | 2-ethylhexyl 4- methoxycinnamate; octyl methoxy cinnamate | 5466-77-3 |
| 32 | 2-ethylhexyl salicylate | 118-60-5 |
| 33 | Benzoic acid, 4,4′,4″-(1,3,5- triazine-2,4,6-triyltriimino)tris(2- | 88122-99-0 |
| | ethylhexyl)ester; 2,4,6-Trianilino-(p-carbo-2′-ethylhexyl-1′-oxi)-1,3,5- | |
| | triazine; octyl triazone | |
| 34 | 4-aminobenzoic acid | 150-13-0 |
| 35 | Benzoic acid, 4-amino-, ethyl ester, polymer with oxirane | 113010-52-9 |
| 38 | 2-phenyl-1H-benzimidazole-5-sulphonic acid; phenylbenzimida- | 27503-81-7 |
| | zolsulfonic acid | |
| 39 | 2-Propenamide, N-[[4-[(4,7,7-trimethyl-3-oxobicyclo[2.2.1]hept-2- | 147897-12-9 |
| | ylidene)methyl]phenyl]methyl]-, homopolymer | |
| 40 | Triethanolamine salicylate | 2174-16-5 |
| 41 | 3,3′-(1,4-phenylenedimethylene)bis[7,7-dimethyl-2-oxo-bicy- | 90457-82-2 |
| | clo[2.2.1]heptane-1 methanesulfonic acid]; Cibafast H | |
| 42 | Titanium dioxide | 13463-67-7 |
| 44 | Zinc oxide | 1314-13-2 |
| 45 | 2,2′-Methylene-bis-[6-(2H-benzotriazol-2-yl)-4-(1,1,3,3-tetramethyl- | 103597-45-1 |
| | butyl)-phenol]; Tinosorb M | |
| 46 | 1H-Benzimidazole-4,6-disulfonic acid, 2,2′-(1,4-phenylene)bis-, | 180898-37-7 |
| | disodium salt | |
| 47 | Benzoic acid, 4,4′-[[6-[[4-[[(1,1- | 154702-15-5 |
| | dimethylethyl)amino]carbonyl]phenyl]amino]1,3,5-triazine-2,4- | |
| | diyl]diimino]bis-, bis(2-ethylhexyl)ester; diethylhexyl butamido tria- | |
| | zone; Uvasorb HEB | |
| 48 | Phenol, 2-(2 H-benzotriazol-2-yl)-4-methyl-6-[2-methyl-3-[1,3,3,3- | 155633-54-8 |
| | tetramethyl-1-[(trimethylsilyl)oxy]disiloxanyl]propyl]-; drometrizole | |
| | trisiloxane; Mexoryl XL | |
| 49 | Dimethicodiethylbenzalmalonate; Polysilicone 15; Parsol SLX | 207574-74-1 |
| 50 | Benzenesulfonic acid, 3-(2H-benzotriazol-2-yl)-4-hydroxy-5-(1- | 92484-48-5 |
| | methylpropyl)-, monosodium salt; Tinogard HS | |
| 51 | Benzoic acid, 2-[4-(diethylamino)-2-hydroxybenzoyl]-, hexyl ester; | 302776-68-7 |
| | Uvinul a plus | |
| 52 | 1-Dodecanaminium, N-[3-[[4-(dimethylamino)benzoyl]amino]propyl]- | 156679-41-3 |
| | N,N-dimethyl-, salt with 4-methylbenzenesulfonic acid (1:1); Escalol | |
| | HP610 | |
| 53 | 1-Propanaminium, N,N,N-trimethyl-3-[(1-oxo-3-phenyl-2-propenyl)- | 177190-98-6 |
| | amino]-, chloride | |
| 54 | 1H-Benzimidazole-4,6-disulfonic acid, 2,2′-(1,4-phenylene)bis- | 170864-82-1 |
| 55 | 1,3,5-Triazine, 2,4,6-tris(4-methoxyphenyl)- | 7753-12-0 |
| 56 | 1,3,5-Triazine, 2,4,6-tris[4-[(2-ethylhexyl)oxy]phenyl]- | 208114-14-1 |
| 57 | 1-Propanaminium, 3-[[3-[3-(2H-benzotriazol-2-yl)-5-(1,1- | 340964-15-0 |
| | dimethylethyl)-4-hydroxyphenyl]-1-oxopropyl]amino]-N,N-diethyl-N- | |
| | methyl-, methyl sulfate (salt) | |
| 58 | 2-Propenoic acid, 3-(1H-imidazol-4-yl)- | 104-98-3 |
| 59 | Benzoic acid, 2-hydroxy-, [4-(1-methylethyl)phenyl]methyl ester | 94134-93-7 |
| 60 | 1,2,3-Propanetriol, 1-(4-aminobenzoate); glyceryl PABA | 136-44-7 |
| 61 | Benzeneacetic acid, 3,4-dimethoxy-a-oxo- | 4732-70-1 |
| 62 | 2-Propenoic acid, 2-cyano-3,3-diphenyl-, ethyl ester | 5232-99-5 |
| 63 | Anthralinic acid, p-menth-3-yl ester | 134-09-8 |
| 64 | 2,2′-bis(1,4-phenylene)-1H-benzimidazole-4,6-disulphonic acid mo- | 349580-12-7, |
| | no sodium salt or Disodium phenyl dibenzimidazole tetrasulfonate or | |
| | Neoheliopan AP | |
| 65 | 1,3,5-Triazine-2,4,6-triamine, N,N′-bis[4-[5-(1,1-dimethylpropyl)-2- | 288254-16-0 |
| | benzoxazolyl]phenyl]-N″-(2-ethylhexyl)- or Uvasorb K2A | |
| 66 | Merocyanine derivatives as described in WO 2004006878 and in | |
| | IPCOM000022279D | |
| 67 | 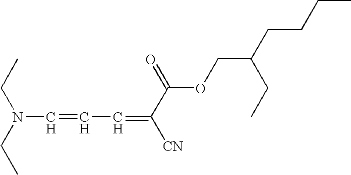 | |
| 68 | sterols (cholesterol, lanosterol, phytosterols), as described in | |
| | WO0341675 | |
| 69 | mycosporines and/or mycosporine-like amino acids as described in | |
| | WO2002039974, e.g. Helioguard 365 from Milbelle AG, isolated my- | |
| | cosporine like amino acids from the red alga porphyra umbilicalis | |
| | (INCI: Porphyra Umbilicalis) that are encapsulated into liposomes,) | |
| 70 | alpha-lipoic-acid as described in DE 10229995 | |
| 71 | synthetic organic polymers as described in EP 1371358, [0033]- | |
| | [0041] | |
| 72 | phyllosilicates as described in EP 1371357 [0034]-[0037] | |
| 73 | silica compounds as described in EP1371356, [0033]-[0041] | |
| 74 | inorganic particles as described in DE10138496 [0043]-[0055] | |
| 75 | latex particles as described in DE10138496 [0027]-[0040] | |
| 76 | 1H-Benzimidazole-4,6-disulfonic acid, 2,2′-(1,4-phenylene)bis-, | 180898-37-7 |
| | disodium salt; Bisimidazylate; Neo Heliopan APC | |
| 77 | 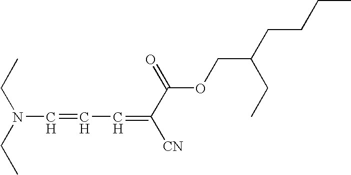 | |
| 78 | 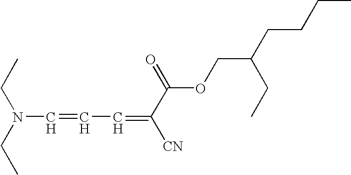 | |
| 79 | 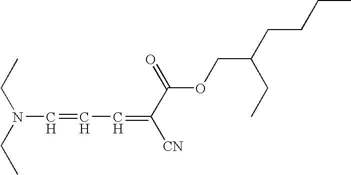 | |
| 80 | 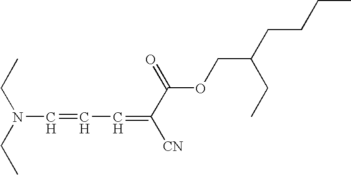 | |
| 81 | 2,4,6-Tris(p-biphenylyl)-s-triazine | 31274-51-8 |
| 82 | 2,4,6-Tris-1,1′,4′,1″-terphenyl-4-yl-1,3,5-triazine | |
| 83 | Di-2-ethylhexyl-3,5-dimethoxy-4-hydroxy-benzalmalonate (Oxynex | |
| | ST, EMD Chemicals, as described in US 20040247536) | |

The composition of the present invention preferably comprises

20 to 70% of component (a),
25 to 75% b.w. of component (b), and
5 to 20% b.w. of component (c),
based on the total weight of the UV absorber composition.

Furthermore, the present invention relates to a cosmetic end-product composition, which preferably comprises

1 to 50% of component (a),
5 to 60% b.w. of component (b), and
20 to 90% b.w. of component (c),
based on the total weight of the UV absorber composition.

Dimethyl capramide (component (b)) is preferably used for enhancing the solubility of the organic UV absorbers present in the oil phase of the UV absorber composition of the present invention and for enhancing the distribution of organic UV absorbers.

Most preferably dimethyl capramide is used for enhancing the solubility and the distribution within the hydro-lipidic film left on the skin of organic UV absorbers of formula (1).

Nevertheless, additional solubilizing agents such as lipophilic solvents may be added to component (b) as single solubilizers or mixture thereof.

Examples of additional solubilizing emollients are all solvents that have good dissolving properties for organic UV absorbers and that are represented by lipophilic esters such as benzoate esters and derivatives; phenylethyl benzoate, ethylhexyl benzoate, C₁₆₋₁₇ alkyl benzoate or any other aliphatic benzoic acids.

Further examples are any compounds with high content of aromatic groups in their molecule like the compounds of general formula:

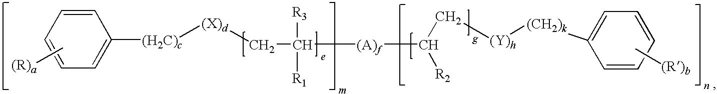

wherein
R and R′ independently from each other are hydrogen; C₁-C₅alkyl; or C₁-C₅alkoxy;
R₁, R₂ and R₃ independently from each other are hydrogen; or C₁-C₅alkyl;
X and Y independently from each other are —O—; —OC(O)—; or —(O)C—O—;
A is A_{α} which is —O—C(O)—O—; A_{β} which is R₄—CH₂C(CH₂—)₃; or A_{γ}, which is —C(H)-isopropyl, where R₄═R_{4δ}═—CH₃; R_{4ε}═—[CH₂CHR₁]ₑ—(Y_{d})—(CH₂)_{c}-Ph(R)ₐ;
a and b independently from each other are a number from 1 to 5;
c and k independently from each other are a number from 0 to 5;
d and h independently from each other are 0; or 1;
e and g independently from each other are 0; or 1;
f is 0; or 1;
m is a number from 1 to 3; and
n is 0; or 1,
or diesters or polyesters of a naphthalene dicarboxylic acid selected from the group consisting of compounds of formula

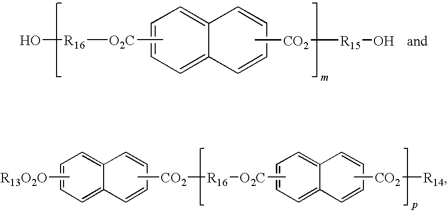

wherein
R₁₃ and R₁₄ independently from each other are C₁-C₂₂alkyl; diols having the structure HO—R₁₆—OH, and polyglycols having the structure HO—R₁₆—(—O—R₁₅)ₙ—OH; wherein R₁₅ and R₁₆ independently from each other are C₁-C₆alkyl; and wherein
m and n are in the range of 1 to 100; and
p is in the range from 0 to 100.
and/or combinations thereof.

Preferred are also pyrrolidone-based surfactants of formula

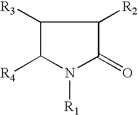

wherein
R₁ is substituted or unsubstituted C₆-C₁₂alkyl;
R₂, R₃ and R₄ independently from each other hydrogen; substituted or unsubstituted C₁-C₄alkyl, preferably hydrogen; unsubstituted C₁-C₂alkyl or substituted C₁-C₂alkyl, and more preferably hydrogen.

All alkyl groups present may be linear or branched.

Suitable substituents for the alkyl groups are for example Cl, Br, F, OH, CN, SO₃H, COOH, NH₂ and N(alkyl)₂.

A preferred N-alkyl substituted pyrrolidone is a pyrrolidone compound of formula

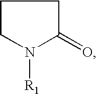

wherein
R is a hydrophobic C₁-C₂₀alkyl.

The main categories of lipophilic emollients are represented as follows:

### Esters:

Esters (includes only esters not otherwise classified as alkoxylated carboxylic acids, glyceryl esters, isethionates, lanolin derivatives, phosphorus compounds, sulfosuccinates or sulfuric acid esters).

Chemically, esters are the covalent compounds formed between acids and alcohols. Esters can be formed from almost all acids (inorganic and carboxylic) and any alcohol. Thus, the number of naturally occurring and synthetically prepared esters used in cosmetics is very large. In order to subdivide the list of esters, most have been assigned to other chemical classes on the basis of some additional chemical features: Alkoxylated fatty acids, alkyl ether sulfates, alkyl sulfates, fats and oils, glyceryl esters, phosphorus compounds, isethionates, sorbitan derivatives, sulfosuccinates, sulfuric acid derivatives, and waxes.

The esters that belong to this specific class are derived from carboxylic acids and have the class RCO—OR′ wherein RCO— represents the carboxylic acid radical and wherein —OR′ is the alcohol residue. Included are low and medium molecular weight compounds, e.g., butyl acetate or isopropyl myristate; some polymers, e.g., polyvinyl Acetate. They are subject to hydrolysis into alcohols and acids at extreme pHs and at elevated temperatures.

Esters in this group find diverse uses in cosmetics as emollients and conditioners (isopropyl myristate), as solvents (ethyl acetate), as fragrance components (methyl salicylate), and as preservatives (propylparaben).

Examples are: acetyl triethylhexyl citrate, acetyl trihexyl citrate, acetylated hydrogenated castor oil, acetylated sucrose distearate, arachidyl erucate, arachidyl propionate, ascorbyl tetraisopalmitate, ascorbyl tocopheryl maleate, behenyl benzoate, benzyl laurate, benzyl ursolate, bis(trioctyldodecyl citryl)dilinoleate, bis-behenyl/isostearyl/phytosteryl dimer dilinoleyl dimer dilinoleate, bis-behenyl/phytosteryl dimer dilinoleate, bis-isostearyl dimer dilinoleyl dimer dilinoleate, butoxyethyl stearate, butyl acetyl ricinoleate, butyl babassuate, butyl isostearate, butyl myristate, butyl oleate, butyl stearate, butylene glycol behenate, butylene glycol laurate, butylene glycol myristate, butylene glycol oleate, butylene glycol palmitate, butylene glycol stearate, butyloctyl benzoate, c10-40 isoalkyl acid phytosterol esters, c11-15 pareth-12 stearate, c11-15 pareth-3 oleate, c11-15 pareth-3 stearate, c12-13 alkyl ethylhexanoate, c12-13 alkyl lactate, c12-15 alkyl benzoate, c12-15 alkyl ethylhexanoate, c12-15 alkyl lactate, c12-15 pareth-12 oleate, c12-15 pareth-3 benzoate, c12-15 pareth-9 hydrogenated tallowate, c14-15 dialkyl carbonate, c14-16 glycol palmitate, c14-18 acid glycol ester, c14-18 alkyl ethylhexanoate, c16-17 alkyl benzoate, c18-20 glycol isostearate, c18-28 alkyl acetate, c18-30 acid glycol ester, c18-36 acid glycol ester, c20-30 glycol isostearate, c23-43 acid pentaerythritol tetraester, c32-36 isoalkyl stearate, castor oil benzoate, ceteareth-6 olivate, cetearyl ethylhexanoate, cetearyl isononanoate, cetearyl palmitate, cetyl acetate, cetyl babassuate, cetyl c12-15 pareth-8 carboxylate, cetyl c12-15-pareth-9 carboxylate, cetyl caprate, cetyl caprylate, cetyl dimethyloctanoate, cetyl ethylhexanoate, cetyl glycol isostearate, cetyl isononanoate, cetyl kombo butterate, cetyl lactate, cetyl laurate, cetyl oleate, cetyl PPG-2 isodeceth-7 carboxylate, chimyl isostearate, chimyl stearate, cholesteryl succinate, coco-caprylate/caprate, coco-rapeseedate, cycloartenyl ferulate, decyl castorate, decyl hempseedate, decyl isostearate, decyl jojobate, decyl oleate, decyl palmitate, decyl succinate, dibutyl adipate, dibutyl sebacate, dibutyldecyl ipdi, dibutyloctyl ipdi, dibutyloctyl malate, dibutyloctyl sebacate, di-c12-13 alkyl malate, di-c12-13 alkyl tartrate, di-c12-15 alkyl adipate, di-c12-15 alkyl fumarate, di-c12-15 alkyl maleate, di-c14-15 alkyl tartrate, dicaprylyl carbonate, dicaprylyl maleate, dicetyl adipate, dicocoyl pentaerythrityl distearyl citrate, diethyl adipate, diethyl palmitoyl aspartate, diethyl sebacate, diethylene glycol diethylhexanoate/diisononanoate, diethylene glycol diisononanoate, diethylhexyl 2,6-naphthalate, diethylhexyl adipate, diethylhexyl carbonate, diethylhexyl fumarate, diethylhexyl malate, diethylhexyl maleate, diethylhexyl succinate, diglyceryl sebacate/isopalmitate, diglyceryl stearate malate, diheptylundecyl adipate, dihexyl adipate, dihexyldecyl adipate, dihexyldecyl ipdi, dihexyldecyl lauroyl glutamate, dihexyldecyl sebacate, dihydrocholesteryl butyrate, dihydrocholesteryl isostearate, dihydrocholesteryl macadamiate, dihydrocholesteryl nonanoate, dihydrocholesteryl octyldecanoate, dihydrocholesteryl oleate, diisoamyl malate, diisobutyl adipate, diisocetyl adipate, diisocetyl dodecanedioate, diisodecyl adipate, diisononyl adipate, diisooctyl adipate, diisopropyl adipate, diisopropyl dimer dilinoleate, diisopropyl sebacate, diisostearyl adipate, diisostearyl fumarate, diisostearyl glutarate, diisostearyl malate, diisostearyl sebacate, dilauryl citrate, dimer dilinoleyl diisostearate, dimer dilinoleyl dimer dilinoleate, dimer dilinoleyl isostearate, dimethicone peg-7 avocadoate, dimethicone peg-7 cocoate, dimethicone peg-7 isostearate, dimethicone peg-7 octyldodecyl citrate, dimethicone peg-7 olivate, dimethicone peg-7 phthalate, dimethicone peg-8 adipate, dimethicone peg-8 beeswax, dimethicone peg-8 borageate, dimethicone peg-8 meadowfoamate, dimethiconol borageate, dimethiconol dhupa butterate, dimethiconol illipe butterate, dimethiconol kokum butterate, dimethiconol lactate, dimethiconol meadowfoamate, dimethiconol mohwa butterate, dimethiconol sal butterate, dimethyl adipate, dimethyl succinate, dimyristyl tartrate, dioctadecanyl didecyltetradecanoate, dioctadecanyl ditetradecyloctadecanoate, dioctyldodeceth-2 lauroyl glutamate, dioctyldodeceth-5 lauroyl glutamate, dioctyldodecyl adipate, dioctyldodecyl lauroyl glutamate, dioctyldodecyl sebacate, dipentaerythrityl hexahydroxystearate/hexastearate/hexahydrogenated rosinate, dipentaerythrityl pentaisononanoate, dipentaerythrityl pentaisostearate, di-PPG-2 myreth-10 adipate, di-PPG-3 ceteth-4 adipate, di-PPG-3 myristyl ether adipate, dipropyl adipate, dipropylene glycol caprylate, dipropylene glycol dibenzoate, dipropylene glycol salicylate, disteareth-2 lauroyl glutamate, disteareth-5 lauroyl glutamate, distearyl citrate, ditridecyl adipate, ditrimethylolpropane isostearate/sebacate, ditrimethylolpropane myristate/stearate, ditrimethylolpropane triethylhexanoate, erythrityl triethylhexanoate, ethoxydiglycol behenate, ethoxydiglycol isostearate, ethoxydiglycol oleate, ethyl almondate, ethyl apricot kernelate, ethyl arachidonate, ethyl avocadate, ethyl caprate, ethyl isostearate, ethyl jojobate, ethyl laurate, ethyl linoleate, ethyl linolenate, ethyl macadamiate, ethyl minkate, ethyl morrhuate, ethyl myristate, ethyl oleate, ethyl olivate, ethyl palmitate, ethyl pelargonate, ethyl persate, ethyl ricinoleate, ethyl stearate, ethyl turtlate, ethyl undecylenate, ethyl wheat germate, ethylhexyl acetoxystearate, ethylhexyl adipate/palmitate/stearate, ethylhexyl benzoate, ethylhexyl benzyldioxopiperazyl acetate, ethylhexyl caprylate/caprate, ethylhexyl cocoate, ethylhexyl ethylhexanoate, ethylhexyl hydroxystearate, ethylhexyl hydroxystearate benzoate, ethylhexyl hydroxystearoyl hydroxystearate, ethylhexyl isononanoate, ethylhexyl isopalmitate, ethylhexyl isostearate, ethylhexyl laurate, ethylhexyl linoleoyl stearate, ethylhexyl myristate, ethylhexyl neopentanoate, ethylhexyl oleate, ethylhexyl palmitate, ethylhexyl pelargonate, ethylhexyl stearate, glycereth-5 lactate, glycereth-7 benzoate, glycereth-7 diisononanoate, glycereth-7 lactate, glyceryl stearate dimer dilinoleate copolymer, glyceryl triacetyl hydroxystearate, glyceryl triacetyl ricinoleate, glycol cetearate, glycol dipalmate/palm kernelate/olivate/macadamiate, glycol dipivalate, glycol ethylhexanoate, glycol hydroxystearate, glycol montanate, glycol oleate, glycol palmitate, glycol ricinoleate, glycol stearate, glycol tallate, glycol/butylene glycol montanate, heptylundecyl hydroxystearate, hexanediol beeswax, hexanediol disalicylate, hexanetriol beeswax, hexyl isostearate, hexyl laurate, hexyldecyl benzoate, hexyldecyl ethylhexanoate, hexyldecyl hexyldecanoate, hexyldecyl laurate, hexyldecyl myristoyl methylaminopropionate, hexyldecyl stearate, hydrogenated ethylhexyl olivate, hydrogenated ethylhexyl sesamate, hydroxycetyl isostearate, hydroxypropyl dimethicone behenate, isoamyl laurate, isobutyl myristate, isobutyl palmitate, isobutyl pelargonate, isobutyl stearate, isobutyl tallowate, isocetyl ethylhexanoate, isocetyl isodecanoate, isocetyl isostearate, isocetyl laurate, isocetyl linoleoyl stearate, isocetyl palmitate, isocetyl stearate, isodeceth-2 cocoate, isodecyl citrate, isodecyl cocoate, isodecyl ethylhexanoate, isodecyl hydroxystearate, isodecyl isononanoate, isodecyl laurate, isodecyl myristate. isodecyl neopentanoate, isodecyl oleate, isodecyl palmitate, isodecyl stearate, isohexyl caprate, isohexyl laurate, isohexyl neopentanoate, isohexyl palmitate, isomerized jojoba oil, isononyl ferulate, isononyl isononanoate, isopropyl arachidate, isopropyl avocadate, isopropyl babassuate, isopropyl behenate, isopropyl c12-15-pareth-9 carboxylate, isopropyl hydroxystearate, isopropyl isostearate, isopropyl jojobate, isopropyl laurate, isopropyl linoleate, isopropyl myristate, isopropyl oleate, isopropyl palmitate, isopropyl PPG-2 isodeceth-7 carboxylate, isopropyl ricinoleate, isopropyl stearate, isopropyl tallowate, isosorbide dicaprylate, isosorbide laurate, isosteareth-10 stearate, isostearyl acetate, isostearyl acetyl glutaminate, isostearyl avocadate, isostearyl benzoate, isostearyl ethylhexanoate, isostearyl ferulate. isostearyl glycolate, isostearyl hydroxystearate, isostearyl isononanoate, isostearyl isostearate, isostearyl lactate, isostearyl laurate, isostearyl linoleate, isostearyl myristate, isostearyl neopentanoate, isostearyl palmitate, isotridecyl isononanoate, isotridecyl stearate, kojic dipalmitate, kojic isopalmitate, laneth-10 acetate, laneth-9 acetate, laureth-2 acetate, laureth-2 benzoate, laureth-2 ethylhexanoate, lauryl cocoate, lauryl ethylhexanoate, lauryl isostearate, lauryl lactate, lauryl-gluconamide palmitates, lignoceryl erucate, linoleyl lactate, meadowfoam estolide, methyl acetyl ricinoleate, methyl behenate, methyl benzoate, methyl caproate, methyl caprylate, methyl caprylate/caprate, methyl cocoate, methyl dehydroabietate, methyl gluceth-20 benzoate, methyl glucose caprylate/caprate, methyl glucose dioleate, methyl glucose isostearate, methyl glucose laurate, methyl glucose sesquicaprylate/sesquicaprate, methyl glucose sesquicocoate, methyl glucose sesquiisostearate, methyl glucose sesquilaurate, methyl glucose sesquioleate, methyl glucose sesquistearate, methyl hydrogenated rosinate, methyl hydroxystearate, methyl isostearate, methyl laurate, methyl linoleate, methyl myristate, methyl oleate, methyl palmate, methyl palmitate, methyl pelargonate, methyl ricinoleate, methyl rosinate, methyl soyate, methyl stearate, methyl sunflowerseedate, monoglycerides acetate, monoglycerides citrate, monoglycerides lactate, monoglycerides succinate, monoglycerides/diacetyl tartarate esters, myreth-2 myristate, myreth-3 caprate, myreth-3 ethylhexanoate, myreth-3 laurate, myreth-3 myristate, myreth-3 palmitate, myristyl acetate, myristyl alaninate, myristyl ethylhexanoate, myristyl isostearate, myristyl lactate, myristyl lignocerate, myristyl neopentanoate, myristyl propionate, neopentyl glycol dicaprate, neopentyl glycol dicaprylate/dicaprate, neopentyl glycol dicaprylate/dipelargonate/dicaprate, neopentyl glycol diethylhexanoate, neopentyl glycol diheptanoate, neopentyl glycol dilaurate, nonyl acetate, octyldodecyl beeswax, octyldodecyl benzoate, octyldodecyl ethylhexanoate, octyldodecyl lactate, octyldodecyl neodecanoate, octyldodecyl neopentanoate, octyldodecyl pca, octyldodecyl/PPG-3 myristyl ether dimer dilinoleate, oleth-2 benzoate, oleuropein aglycone, oleyl acetate, oleyl lactate, oleyl oleate, palmitoyl inulin, peg-20 methyl glucose distearate, peg-20 methyl glucose sesquicaprylate/sesquicaprate, peg-20 methyl glucose sesquilaurate, peg-3 butylene glycol laurate, peg-3 trimethylolpropane tristearate, peg-4 trimethylolpropane distearate, peg-5 tricapryl citrate, peg-5 tricetyl citrate, peg-5 trilauryl citrate, peg-5 trimethylolpropane trimyristate, peg-5 trimyristyl citrate, peg-5 tristearyl citrate, peg-6 butylene glycol laurate, peg-6 propylene glycol caprylate/caprate, peg-8 propylene glycol cocoate, peg-80 methyl glucose laurate, pentaerythrityl dioleate, pentaerythrityl distearate, pentaerythrityl rosinate, pentaerythrityl stearate, pentaerythrityl tetrabehenate/benzoate/ethylhexanoate, pentaerythrityl tetrabenzoate, pentaerythrityl tetraethylhexanoate/benzoate, pentaerythrityl tetraethylhexanoate/tetramethoxycinnamate, pentaerythrityl trioleate, phenethyl benzoate, phytosteryl hydroxystearate, phytosteryl isostearyl dimer dilinoleate, phytosteryl rice branate, poloxamer 182 dibenzoate, polyperfluoroethoxymethoxy difluoroethyl peg diisostearate, PPG-15 stearyl ether benzoate, PPG-2 isoceteth-20 acetate, PPG-20 methyl glucose ether distearate, PPG-3 dipivalate, PPG-3 myristyl ether neoheptanoate, propyl c12-15 pareth-8 carboxylate, propylene glycol behenate, propylene glycol caprylate, propylene glycol ceteth-3 acetate, propylene glycol ceteth-3 propionate, propylene glycol cocoate, propylene glycol dibenzoate, propylene glycol heptanoate, propylene glycol hydroxystearate, propylene glycol isoceteth-3 acetate, propylene glycol isostearate, propylene glycol laurate, propylene glycol linoleate, propylene glycol linolenate, propylene glycol myristate, propylene glycol myristyl ether acetate, propylene glycol oleate, propylene glycol ricinoleate, propylene glycol soyate, propylene glycol stearate, quercetin caprylate, raffinose myristate, raffinose oleate, retinyl formyl aspartamate, salicylyl beeswax, starch tallowate, steareth-9 stearate, stearyl acetate, stearyl acetyl glutaminate, stearyl benzoate, stearyl citrate, stearyl glycol isostearate, stearyl lactate, stearyl/PPG-3 myristyl ether dimer dilinoleate, sucrose acetate/stearate, sucrose cocoate, sucrose dilaurate, sucrose distearate, sucrose hexaerucate, sucrose laurate, sucrose mortierellate, sucrose myristate. sucrose oleate, sucrose palmitate, sucrose pentaerucate, sucrose polybehenate, sucrose polycottonseedate, sucrose polylaurate, sucrose polylinoleate, sucrose polyoleate, sucrose polypalmate, sucrose polysoyate, sucrose polystearate, sucrose ricinoleate, sucrose stearate, sucrose tetraisostearate, sucrose tetrastearate triacetate, sucrose tribehenate, sucrose tristearate, tetradecylpropionates, tetrahydrofurfuryl ricinoleate, tocopheryl retinoate, tribenzoyl triricinolein, tri-c12-13 alkyl citrate, tri-c14-15 alkyl citrate, tricaprylyl/capryl trimellitate, tridecyl ethyl hexanoate, tridecyl isononanoate, tridecyl neopentanoate, tridecyl stearate, triethylhexyl citrate, triethylhexyl trimellitate, triisodecyl trimellitate, triisopropyl citrate, trioleyl citrate.

### Glyceryl Esters and Derivatives (Excluding Fats and Oils)

Glyceryl Esters comprise a subgroup of esters that are primarily Fatty Acid mono- and diglycerides or triglycerides modified by reaction with other Alcohols and the like. Also included are the fatty acid esters of polyglycerin, the complex dehydration product of glycerin, e.g., Polyglyceryl-4 Isostearate. Like natural fats and oils, these Glyceryl Esters are subject to hydrolysis. They are used in cosmetics as emollients, lubricants, vehicles, and thickening agents. In addition, the monoglycerides (e.g., Glyceryl Stearate) are important emulsifiers.

acetyl glyceryl ricinoleate, acetylated lard glyceride, acetylated palm kernel glycerides, argan oil peg-8 esters, bis-diglyceryl polyacyladipate-1, bis-diglyceryl polyacyladipate-2, c10-40 isoalkyl acid glyceride, c10-40 isoalkyl acid triglyceride, canola oil glyceride, caprylic/capric glycerides, caprylic/capric/coco glycerides, caprylic/capric/succinic triglyceride, cocoglycerides.

Cottonseed glyceride, datem, di-glyceryl stearate dimer dilinoeate, diglyceryl stearate malate, diisostearoyl polyglyceryl-3 dimer dilinoleate, ethylhexyloxyglyceryl palmitate, fish glycerides, glycereth-18 benzoate, glycereth-20 benzoate, glycereth-6 c10-18 acidate, glycereth-7 triacetate, glycerin/phthalic acid copolymer hydroxypropyl c9-11 isoalkyl acidate, glyceryl abietate, glyceryl abietate/maleate, glyceryl adipate, glyceryl alginate, glyceryl arachidate, glyceryl arachidonate, glyceryl behenate, glyceryl behenate/eicosadioate, glyceryl caprate, glyceryl caprylate, glyceryl caprylate/caprate, glyceryl citrate/lactate/linoleate/oleate, glyceryl cocoate, glyceryl cocoate/citrate/lactate, glyceryl collagenate, glyceryl diarachidate, glyceryl dibehenate, glyceryl dibehenate/hydrogenated rosinate, glyceryl dierucate, glyceryl di-hydroxystearate, glyceryl diisopalmitate, glyceryl diisostearate, glyceryl dilaurate, glyceryl dilinoleate, glyceryl dimyristate, glyceryl dimyristate/hydrogenated rosinate, glyceryl dioleate, glyceryl dipalmitate, glyceryl dipalmitoleate, glyceryl diricinoleate, glyceryl distearate, glyceryl erucate, glyceryl heptanoate, glyceryl hydrogenated rosinate, glyceryl hydrogenated soyate, glyceryl hydroxystearate, glyceryl isopalmitate, glyceryl isostearate, glyceryl isostearate/myristate, glyceryl isostearates, glyceryl isotridecanoate/stearate/adipate, glyceryl lanolate, glyceryl laurate, glyceryl laurate/oleate, glyceryl linoleate, glyceryl linolenate, glyceryl montanate, glyceryl myristate, glyceryl oleate, glyceryl oleate/elaidate, glyceryl olivate, glyceryl palmitate, glyceryl palmitate lactate, glyceryl palmitate/stearate, glyceryl palmitoleate, glyceryl ricinoleate, glyceryl ricinoleate se, glyceryl rosinate, glyceryl sesquioleate, glyceryl stearate, glyceryl stearate citrate, glyceryl stearate lactate, glyceryl stearate succinate, glyceryl stearate/acetate, glyceryl stearate/malate, glyceryl stearates, glyceryl tallowate, glyceryl tripalmate/palm kernelate/olivate/macadamiate/rapeseedate, glyceryl undecylenate, glyceryl/sorbitol oleate/hydroxystearate, hydrogenated castor oil/sebacic acid copolymer, hydrogenated coco-glycerides, hydrogenated lard glyceride, hydrogenated lard glycerides, hydrogenated palm glycerides, hydrogenated palm glycerides citrate, hydrogenated palm kernel glycerides, hydrogenated soy glycerides, hydrogenated tallow glyceride citrate, hydrogenated tallow glyceride lactate, hydrogenated tallow glycerides, hydrogenated tallow glycerides citrate, hydrogenated vegetable glycerides, hydrogenated vegetable glycerides citrate, hydroxylated milk glycerides, hydroxystearic/linolenic/oleic polyglycerides, isostearic/myristic glycerides, lard glycerides, lauryl polyglyceryl-6 cetearyl glycol ether, methylsilanol PEG-7 glyceryl cocoate, methylsilanol tri-PEG-8 glyceryl cocoate, oleic/linoleic/linolenic polyglycerides, palm glycerides, palm kernel glycerides, PEG-10 castor oil, PEG-10 glyceryl triisostearate, PEG-10 glyceryl trioleate, PEG-10 glyceryl tristearate, PEG-10 hydrogenated castor oil, PEG-10 olive glycerides, PEG-10 sunflower glycerides, PEG-11 avocado glycerides, PEG-11 babassu glycerides, PEG-11 castor oil, PEG-11 cocoa butter glycerides, PEG-12 glyceryl dioleate, PEG-12 glyceryl distearate, PEG-12 palm kernel glycerides, PEG-13 mink glycerides, PEG-13 Sunflower Glycerides, PEG-14 avocado glycerides, PEG-15 castor oil, PEG-15 glyceryl laurate, PEG-15 glyceryl triisostearate, PEG-15 glyceryl trioleate, PEG-16 castor oil, PEG-16 hydrogenated castor oil, PEG-16 macadamia glycerides, PEG-18 glyceryl oleate/cocoate, PEG-18 palm glycerides, PEG-192 apricot kernel glycerides, PEG-2 castor oil, PEG-2 hydrogenated castor oil, PEG-2 olive glycerides, PEG-2 sunflower glycerides, PEG-20 almond glycerides, PEG-20 corn glycerides, PEG-20 evening primrose glycerides, PEG-20 glyceryl stearate, PEG-20 glyceryl triisostearate, PEG-20 glyceryl tristearate, PEG-20 hydrogenated palm glycerides, PEG-25 glyceryl trioleate, PEG-25 moringa glycerides, PEG-3 castor oil, PEG-3 glyceryl cocoate, PEG-3 glyceryl triisostearate, PEG-3 glyceryl trioleate, PEG-3 glyceryl tristearate, PEG-30 glyceryl triisostearate, PEG-35 almond glycerides, PEG-4 caprylic/capric glycerides, PEG-4 castor oil, PEG-4 glyceryl distearate, PEG-4 glyceryl tristearate, PEG-40 glyceryl triisostearate, PEG-40 olive glycerides, PEG-42 babassu glycerides, PEG-42 mushroom glycerides, PEG-45 palm kernel glycerides, PEG-45 safflower glycerides, PEG-5 castor oil, PEG-5 glyceryl triisostearate, PEG-5 glyceryl trioleate, PEG-5 glyceryl tristearate, PEG-5 hydrogenated castor oil, PEG-5 hydrogenated corn glycerides, PEG-50 glyceryl triisostearate, PEG-6 almond glycerides, PEG-6 caprylic/capric glycerides, PEG-6 glyceryl caprate, PEG-6 glyceryl tristearate, PEG-6 hydrogenated castor oil, PEG-6 hydrogenated palm/palm kernel glyceride, PEG-6 olive glycerides, PEG-60 almond glycerides, PEG-60 corn glycerides, PEG-60 glyceryl triisostearate, PEG-60 shea butter glycerides, PEG-65 hydrogenated castor oil, PEG-7 caprylic/capric glycerides, PEG-7 glyceryl cocoate, PEG-7 glyceryl laurate, PEG-7 glyceryl soyate, PEG-7 hydrogenated castor oil, PEG-7 olive glycerides, PEG-7 sunflower glycerides, PEG-70 mango glycerides, PEG-75 cocoa butter glycerides, PEG-75 meadowfoam oil, PEG-75 shea butter glycerides, PEG-75 shorea butter glycerides, PEG-75 soy glycerides, PEG-8 caprylic/capric glycerides, PEG-8 castor oil, PEG-8 glyceryl laurate, PEG-8 hydrogenated castor oil, PEG-8 hydrogenated fish glycerides, PEG-9 castor oil, PEG-9 cocoglycerides, polyglyceryl-10 caprate, polyglyceryl-10 decahydroxystearate, polyglyceryl-10 decaisostearate, polyglyceryl-10 decalinoleate, polyglyceryl-10 decamacadamiate, polyglyceryl-10 decaoleate, polyglyceryl-10 decastearate, polyglyceryl-10 didecanoate, polyglyceryl-10 diisostearate, polyglyceryl-10 dioleate, polyglyceryl-10 dipalmitate, polyglyceryl-10 distearate, polyglyceryl-10 dodecacaprate, polyglyceryl-10 dodecacaprylate, polyglyceryl-10 heptaoleate, polyglyceryl-10 heptastearate, polyglyceryl-10 hexaoleate, polyglyceryl-10 hydroxystearate/stearate/eicosadioate, polyglyceryl-10 isostearate, polyglyceryl-10 mono/dioleate, polyglyceryl-10 nonaerucate, polyglyceryl-10 nonaisostearate, polyglyceryl-10 palmitate, polyglyceryl-10 pentahydroxystearate, polyglyceryl-10 pentaisostearate, polyglyceryl-10 pentalaurate, polyglyceryl-10 pentalinoleate, polyglyceryl-10 pentaoleate, polyglyceryl-10 pentaricinoleate, polyglyceryl-10 pentastearate, polyglyceryl-10 tetraoleate, polyglyceryl-10 tridecanoate, polyglyceryl-10 tristearate, polyglyceryl-2 caprate, polyglyceryl-2 caprylate, polyglyceryl-2 diisostearate, polyglyceryl-2 dioleate, polyglyceryl-2 isopalmitate, polyglyceryl-2 isostearate, polyglyceryl-2 laurate, polyglyceryl-2 myristate, polyglyceryl-2 oleate, polyglyceryl-2 palmitate, polyglyceryl-2 sesquicaprylate, polyglyceryl-2 sesquiisostearate, polyglyceryl-2 sesquioleate, polyglyceryl-2 sesquistearate, polyglyceryl-2 stearate, polyglyceryl-2 tetraisostearate, polyglyceryl-2 tetrastearate, polyglyceryl-2 triisostearate, polyglyceryl-3 caprate, polyglyceryl-3 dicaprate, polyglyceryl-3 dicocoate, polyglyceryl-3 di-hydroxystearate, polyglyceryl-3 diisostearate, polyglyceryl-3 dioleate, polyglyceryl-3 distearate, polyglyceryl-3 isostearate, polyglyceryl-3 laurate, polyglyceryl-3 methylglucose distearate, polyglyceryl-3 myristate, polyglyceryl-3 oleate, polyglyceryl-3 palmitate, polyglyceryl-3 pentaricinoleate, polyglyceryl-3 ricinoleate, polyglyceryl-3 stearate, polyglyceryl-3 triisostearate, polyglyceryl-4 caprate, polyglyceryl-4 dilaurate, polyglyceryl-4 isostearate, polyglyceryl-4 laurate, polyglyceryl-4 oleate, polyglyceryl-4 pentaoleate, polyglyceryl-4 pentastearate, polyglyceryl-4 tristearate, polyglyceryl-5 caprate, polyglyceryl-5 hexastearate, polyglyceryl-5 isostearate, polyglyceryl-5 laurate, polyglyceryl-5 myristate, polyglyceryl-5 oleate, polyglyceryl-5 pentamyristate, polyglyceryl-5 tribehenate, polyglyceryl-5 trimyristate, polyglyceryl-5 trioleate, polyglyceryl-6 caprylate, polyglyceryl-6 dicaprate, polyglyceryl-6 diisostearate, polyglyceryl-6 dioleate, polyglyceryl-6 dipalmitate, polyglyceryl-6 distearate, polyglyceryl-6 hexaoleate, polyglyceryl-6 hexastearate, polyglyceryl-6 isostearate, polyglyceryl-6 laurate, polyglyceryl-6 myristate, polyglyceryl-6 octastearate, polyglyceryl-6 oleate, polyglyceryl-6 palmitate, polyglyceryl-6 pentaoleate, polyglyceryl-6 pentaricinoleate, polyglyceryl-6 pentastearate, polyglyceryl-6 polyricinoleate, polyglyceryl-6 stearate, polyglyceryl-6 tetrabehenate, polyglyceryl-6 tetraoleate, polyglyceryl-8 decaerucate/decaisostearate/decaricinoleate, PPG-3 hydrogenated castor oil, PPG-5 lanolin wax glyceride, PPG-67 glyceryl ether, PPG-70 glyceryl ether, rapeseed glyceride, rapeseed glycerides, rubus occidentalisseed oil, sunflower seed oil glyceride, sunflower seed oil glycerides, tall oil glycerides, tallow glycerides, tribenzoyl triricinolein, triisostearoyl polyglyceryl-3 dimer dilinoleate, vegetable glycerides phosphate, wheat germ glycerides.

### Alkoxylated Alcohols Esters

C12-15 pareth-3 benzoate, cetyl c12-15 pareth-8 carboxylate, di-PPG-2 myreth-10 adipate, di-PPG-3 ceteth-4 adipate, di-PPG-3 myristyl ether adipate, glycereth-5 lactate, glycereth-7 benzoate, glycereth-7 diisononanoate, glycereth-7 lactate, oleth-2 benzoate, PEG-3 butylene glycol laurate, PEG-5 trilauryl citrate, PEG-5 trimyristyl citrate, PEG-5 tristearyl citrate, PEG-6 butylene glycol laurate, PEG-6 propylene glycol caprylate/caprate, PEG-8 propylene glycol cocoate, PPG-15 stearyl ether benzoate, PPG-20 methyl glucose ether distearate, PPG-3 myristyl ether neoheptanoate, methyl gluceth-20 benzoate.

### Alkoxylated Alcohols Glyceryl Esters and Derivatives

glycereth-18 benzoate, glycereth-20 benzoate, glycereth-7 triacetate, PEG-10 castor oil, PEG-10 hydrogenated castor oil, PEG-10 olive glycerides, PEG-10 sunflower glycerides, PEG-11 avocado glycerides, PEG-11 babassu glycerides, PEG-11 castor oil, PEG-11 cocoa butter glycerides, PEG-12 glyceryl dioleate, PEG-12 glyceryl distearate, PEG-12 palm kernel glycerides, PEG-13 mink glycerides, PEG-13 sunflower glycerides, PEG-14 avocado glycerides, PEG-15 castor oil, PEG-15 glyceryl laurate, PEG-16 castor oil, PEG-16 hydrogenated castor oil, PEG-16 macadamia glycerides, PEG-18 glyceryl oleate/cocoate, PEG-18 palm glycerides, PEG-192 apricot kernel glycerides, PEG-2 castor oil, PEG-2 hydrogenated castor oil, PEG-2 olive glycerides, PEG-2 sunflower glycerides, PEG-20 almond glycerides, PEG-20 corn glycerides, PEG-20 evening primrose glycerides, PEG-20 glyceryl stearate, PEG-20 hydrogenated palm glycerides, PEG-25 moringa glycerides, PEG-3 castor oil, PEG-3 glyceryl cocoate, PEG-4 castor oil, PEG-4 glyceryl distearate, PEG-40 olive glycerides, PEG-42 babassu glycerides, PEG-42 mushroom glycerides, PEG-45 palm kernel glycerides, PEG-45 safflower glycerides, PEG-5 castor oil, PEG-5 hydrogenated castor oil, PEG-5 hydrogenated corn glycerides, PEG-50 glyceryl triisostearate, PEG-6 almond glycerides, PEG-6 glyceryl caprate, PEG-60 almond glycerides, PEG-60 corn glycerides, PEG-60 shea butter glycerides, PEG-65 hydrogenated castor oil, PEG-7 glyceryl cocoate, PEG-7 glyceryl laurate, PEG-7 glyceryl soyate, PEG-7 hydrogenated castor oil, PEG-7 olive glycerides, PEG-7 sunflower glycerides, PEG-70 mango glycerides, PEG-75 cocoa butter glycerides, PEG-75 meadowfoam oil, PEG-75 shea butter glycerides, PEG-75 shorea butter glycerides, PEG-75 soy glycerides, PEG-8 castor oil, PEG-8 glyceryl laurate, PEG-8 hydrogenated castor oil, PEG-8 hydrogenated fish glycerides, PEG-9 castor oil, PEG-9 cocoglycerides, PPG-3 hydrogenated castor oil, PPG-5 lanolin wax glyceride.

### Alkoxylated Alcohol Ethers

PPG-9 diglyceryl ether glycereth-7 trimethyl ether, PPG-10 cetyl ether, PPG-10 oleyl ether, PPG-11 stearyl ether, PPG-14 butyl ether, PPG-15 stearyl ether, PPG-20 cetyl ether, PPG-20 oleyl ether, PPG-23 oleyl ether, PPG-28 cetyl ether, PPG-3 benzyl ether myristate, PPG-3 myristyl ether, PPG-30 cetyl ether, PPG-30 isocetyl ether, PPG-30 oleyl ether, PPG-37 oleyl ether, PPG-3-isodeceth-1, PPG-4 lauryl ether, PPG-4 myristyl ether, PPG-5 pentaerythrityl ether, PPG-50 cetyl ether, PPG-50 oleyl ether, PPG-65-PEG-5 pentaerythrityl ether, PPG-9-steareth-3.

### Fats and Oils

Fats and Oils, as a group, are the glyceryl esters of Fatty Acids (triglycerides) normally found in animal and plant tissues, including those which have been hydrogenated to reduce or eliminate unsaturation. Also included are synthetically-prepared esters of glycerin and fatty acids. They possess the class:

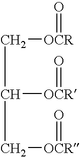

in which —COR, —COR′, —COR″ may be the same or different fatty acid radicals.

Naturally occurring fats and oils in this group are Esters and can be hydrolyzed to yield glycerin and mixtures of fatty acids. These fatty acids may serve as raw materials for a variety of other chemicals useful in cosmetics. Alternately, isolated and purified fatty acids can be esterified with glycerin to yield mono-, di-, and triglycerides. Fats and Oils are used directly in cosmetics as emollient vehicles and lubricants. Volatile oils obtained by the distillation of plant material are classified as Essential Oils.

### With Occlusive Properties

Occlusive are cosmetic ingredients that slow down the evaporation of water from the skin surface. This function is different from Antiperspirant Agents that interfere with the delivery of liquid water to the skin surface. By blocking the evaporative loss of water, occlusive materials increase the water content of skin. Occlusive agents are generally lipids that tend to remain on the skin surface

Argania spinosa kernel oil, argemone mexicana oil, astrocaryum vulgare fruit oil, astrocaryum vulgare kernel oil, avena sativa (oat) kernel oil, balanites roxburghii seed oil, bassia butyracea seed butter, bassia latifolia seed butter, brassica campestris (rapeseed) seed oil, buffalo fat, butter, butyrospermum parkii (shea butter), butyrospermum parkii (shea butter) oil, c10-18 triglycerides, c12-18 acid triglyceride, c8-12 acid triglyceride, calendula officinalis seed oil, calophyllum tacamahaca seed oil, camellia japonica seed oil, camellia kissi seed oil, camellia sinensis seed oil, canola oil, caprylic/capric triglyceride, caprylic/capric/lauric triglyceride, caprylic/capric/linoleic triglyceride, caprylic/capric/myristic/stearic triglyceride, caprylic/capric/stearic triglyceride, carthamus tinctorius (safflower) seed oil, carya illinoensis (pecan) seed oil, caryocar coriaceum seed oil, castor oil benzoate, cibotium barometz oil, citrullus vulgaris (watermelon) seed oil, cocos nucifera (coconut) oil, cod liver oil, cod liver/mink/tallow triglyceride, coix lacryma-jobi (job's tears) seed oil, corylus americana (hazel) seed oil, crambe abyssinica seed oil, cucurbita pepo (pumpkin) seed oil, deer fat, elaeis guineensis (palm) kernel oil, elaeis guineensis (palm) oil, emu oil, euterpe oleracea fruit oil, fish liver oil, garcinia indica seed butter, gevuina avellana oil, glycine soja (soybean) lipids, glycine soja (soybean) oil, goat butter, goat fat, gossypium herbaceum (cotton) seed oil, helianthus annuus (sunflower) seed oil, hippophae rhamnoides oil, horse fat, hydrogenated apricot kernel oil, hydrogenated c12-18 triglycerides, hydrogenated canola oil, hydrogenated castor oil, hydrogenated castor oil laurate, hydrogenated castor oil triisostearate, hydrogenated coconut oil, hydrogenated cottonseed oil, hydrogenated fish oil, hydrogenated lard, hydrogenated menhaden oil, hydrogenated milk lipids, hydrogenated mink oil, hydrogenated olive oil, hydrogenated orange roughy oil, hydrogenated ostrich oil, hydrogenated palm kernel oil, hydrogenated palm oil, hydrogenated peanut oil, hydrogenated pistachio seed oil, hydrogenated rapeseed oil, hydrogenated rice bran oil, hydrogenated safflower seed oil, hydrogenated shark liver oil, hydrogenated shea butter, hydrogenated soybean oil, hydrogenated sunflower seed oil, hydrogenated sweet almond oil, hydrogenated tallow, hydrogenated vegetable oil, hydrogenated wheat germ oil, irvingia gabonensis kernel butter, isatis tinctoria seed oil, juglans regia (walnut) seed oil, kyounin yu, lard, lauric/palmitic/oleic triglyceride, limnanthes alba (meadowfoam) seed oil, linum usitatissimum (linseed) seed oil, luffa cylindrica seed oil, lupinus albus seed oil, macadamia integrifolia seed oil, macadamia ternifolia seed oil, mangifera indica (mango) seed butter, mangifera indica (mango) seed oil, marmot oil, melia azadirachta seed oil, melissa officinalis seed oil, menhaden oil, mink oil, moringa pterygosperma seed oil, mortierella oil, natane yu, neatsfoot oil, nigella sativa seed oil, oenocarpus bacaba fruit oil, oenocarpus bataua fruit oil, olea europaea (olive) fruit oil, olea europaea (olive) husk oil, oleic/linoleic triglyceride, oleic/palmitic/lauric/myristic/linoleic triglyceride, omental lipids, orange roughy oil, orbignya cohune seed oil, orbignya oleifera seed oil, oryza sativa (rice) bran oil, oryza sativa (rice) germ oil, ostrich oil, papaver orientale (poppy) seed oil, passiflora edulis seed oil, persea gratissima (avocado) butter, persea gratissima (avocado) oil, pinus koraiensis seed oil, pistacia vera seed oil, prunus amygdalus dulcis (sweet almond) oil, prunus armeniaca (apricot) kernel oil, prunus avium (sweet cherry) seed oil, prunus domestica seed oil, prunus insititia seed oil, prunus persica (peach) kernel oil, rabbit fat, ricinoleic/caproic/caprylic/capric triglyceride, ricinus communis (castor) seed oil, rosa eglentaria seed oil, salmon oil, salvia hispanica seed oil, santalum album (sandalwood) seed oil, schleichera trijuga seed oil, sesamum indicum (sesame) seed oil, shark liver oil, shorea robusta seed butter, solanum lycopersicum (tomato) fruit oil, solanum lycopersicum (tomato) seed oil, tall oil, tallow, telphairia pedata oil, theobroma cacao (cocoa) seed butter, theobroma grandiflorum seed butter, triarachidin, tribehenin, tricaprin, tricaprylin, trichodesma zeylanicum oil, trierucin, triethylhexanoin, triheptanoin, triheptylundecanoin, rihydroxymethoxystearin, trihydroxystearin, triisononanoin, triisopalmitin, triisostearin, trilaurin, trilinolein, trilinolenin, trimyristin, triolein, tripalmitin, tripalmitolein, triricinolein, trisebacin, tristearin, triticum vulgare (wheat) bran lipids, triticum vulgare (wheat) germ oil, triundecanoin, turtle oil, vaccinium macrocarpon (cranberry) seed oil, vateria indica seed butter, virola sebifera nut oil, zea mays (corn) germ oil, zea mays (corn) oil.

### With Emollient Properties

Emollient are cosmetic ingredients that help to maintain the soft, smooth, and pliable appearance of skin. Emollients function, via their ability to remain on the skin surface or in the stratum corneum, and act as lubricants to reduce flaking, and improve the skin's appearance.

Acetylated hydrogenated castor oil, actinidia chinensis (kiwi) seed oil, adansonia digitata seed oil, argania spinosa kernel oil, argemone mexicana oil, astrocaryum murumuru butter, bertholletia excelsa seed oil, bixa orellana seed oil, borago officinalis seed oil, brain lipids, brassica oleracea italica (broccoli) seed oil, c10-40 isoalkyl acid triglyceride, calodendrum capense nut oil, calophyllum tacamahaca seed oil, camellia kissi seed oil, carica papaya seed oil, caryocar coriaceum seed oil, castor oil benzoate, chenopodium quinoa seed oil, citrullus lanatus (watermelon) seed oil, cod liver/mink/tallow triglyceride, coix lacryma-jobi (job's tears) seed oil, cucumis sativus (cucumber) oil, daucus carota sativa (carrot) seed oil, glyceryl triacetyl hydroxystearate, glyceryl triacetyl ricinoleate, haematococcus pluvialis oil, hydrogenated egg oil, hydrogenated evening primrose oil, hydrogenated grapeseed oil, hydrogenated hazelnut oil, hydrogenated hemp oil, hydrogenated hemp seed oil, hydrogenated macadamia seed oil, hydrogenated sesame seed oil, isatis tinctoria seed oil, lithospermum officinale seed oil, lupinus albus seed oil, lycium barbarum seed oil, mamushi oil, marrow lipids, milk lipids, morinda citrifolia seed oil, mucor circinelloides oil, mustelic/palmitic triglyceride, odontella aurita oil, orbignya speciosa kernel oil, oxycoccus palustris seed oil, papaver somniferum seed oil, parinari curatellifolia seed oil, passiflora edulis seed oil, pinus parviflora seed oil, pinus pentaphylla seed oil, plukenetia volubilis seed oil, prunus insititia seed oil, punica granatum seed oil, pycnanthus angolensis nut oil, pyrus malus (apple) seed oil, ribes nigrum (black currant) seed oil, rosa moschata seed oil, rosa rubiginosa seed oil, rubus glaucus seed oil, rubus idaeus (raspberry) seed oil, schinziophyton rautanenii kernel oil, sclerocarya birrea seed oil, silkworm chrysalis oil, soybean glycerides, spinal cord lipids, sterculia africana seed oil, torreya nucifera seed oil, vaccinium vitis-idaea seed oil, vanilla planifolia fruit oil, virola sebifera nut oil.

### Fatty Alcohols

Fatty Alcohols are higher molecular weight nonvolatile alcohols. They are produced from natural Fats and Oils by reduction of the Fatty Acid —COOH grouping to the hydroxyl function. Alternately, several completely synthetic routes yield fatty alcohols that may be structurally identical or similar to the nature-derived alcohols. Fatty Alcohols generally are primary alcohols conforming to the class RCH₂OH; those fatty alcohols prepared from naturally occurring fatty acids normally contain an even number of carbon atoms.

Fatty Alcohols are used as emollients in numerous types of cosmetics. They are valuable as co-emulsifiers and are employed to increase the viscosity of emulsions, shampoos, and other products. In addition, these alcohols are used for the synthesis of some widely used cosmetic ingredients (e.g., alkoxylated alcohols, alkyl sulfates, alkyl ether sulfates, and phosphorus compounds). arachidyl alcohol, behenyl alcohol, C₉₋₁₁ alcohols, c₁₂₋₁₃ alcohols, .C₁₂₋₁₅ alcohols, .C₁₂₋₁₆ alcohols, .C₁₄₋₁₅ alcohols, .C₁₄₋₂₂ alcohols, .C₂₀₋₂₂ alcohols, .caprylic alcohol, cetearyl alcohol, .cetyl alcohol, coconut alcohol, .C₆₋₁₂ perfluoroalkylethanol, .decyl alcohol, .hydrogenated jojoba alcohol, hydrogenated rapeseed alcohol, hydrogenated tallow alcohol, jojoba alcohol, lauryl alcohol, myristyl alcohol, oleyl alcohol, olive alcohol, palm alcohol, .palm kernel alcohol, stearyl alcohol, tallow alcohol and tridecyl alcohol.

### Ethers

Ethers are the organic compounds possessing a C—O—C system of bonds. If this C—O—C system is part of a ring, the corresponding compound is classified with the heterocyclics, e.g., tocopherol. Sorbitan derivatives are also heterocyclic ethers. Most ethers used in cosmetics are prepared synthetically. Alkoxylated derivatives are ethers, but are herein classed as alkoxylated alcohols, alkoxylated amides, alkoxylated amines, alkoxylated carboxylic acids, and polymeric ethers. The esterified polyglyceryl ethers resulting from the dehydration of glycerin are included in the group of glyceryl esters.

### With Emollient Properties

Emollient are cosmetic ingredients that help to maintain the soft, smooth, and pliable appearance of skin. Emollients function, via their ability to remain on the skin surface or in the stratum corneum, and act as lubricants to reduce flaking, and improve the skin's appearance.

Examples are butoxyethyl stearate, c12-14 hydroxyalkyl hydroxydimerdilinoleyl ether, c12-14 hydroxyalkyl maltitol ether, c16-18 hydroxyalkyl hydroxydimerdilinoleyl ether, cetyl glyceryl ether, cetyloxypropyl glyceryl methoxypropyl myristamide, chimyl alcohol, chimyl isostearate, chimyl stearate, dicaprylyl ether, diretinyl ether, ethoxydiglycol behenate, ethoxydiglycol isostearate, ethoxydiglycol oleate, ethylhexyloxyglyceryl palmitate, eugenyl glucoside, hydrolyzed proanthocyanidin, hydroxyethyl palmityl oxyhydroxypropyl palmitamide, isopolyglycerin-3 dimethicone, isopropyl hydroxycetyl ether, isostearyl ferulate, isostearyl glyceryl ether, isostearyl glyceryl pentaerythrityl ether, lauryl polyglyceryl-6 cetearyl glycol ether, oleyl glyceryl ether, polyglyceryl-2 lanolin alcohol ether, polyglyceryl-2 oleyl ether, polyglyceryl-3 decyltetradecyl ether, polyglyceryl-3 hydroxylauryl ether, polyglyceryl-4 lauryl ether, polyglyceryl-4 oleyl ether, ppg-9 diglyceryl ether, propylene glycol myristyl ether

### With Solvent Properties

Butoxyethyl acetate, diethoxydiglycol, dimethoxydiglycol, dimethyl ether, dimethyl isosorbide, dioxolane, dipropylene glycol, dipropylene glycol dimethyl ether, ethoxydiglycol acetate, ethoxyethanol acetate, ethyl ether, ethyl perfluorobutyl ether, ethyl perfluoroisobutyl ether, methoxydiglycol, methoxyethanol acetate, methoxyisopropyl acetate, methyl hexyl ether, methyl perfluorobutyl ether, methyl perfluoroisobutyl ether, methylal, propylene glycol t-butyl ether.

### Hydrocarbons

Hydrocarbons are the group of compounds containing only carbon and hydrogen. Hydrocarbons are generally derived from petrochemicals, but some of them are found in the plant and animal kingdom (e.g., Azulene or Squalene). Their class can vary widely, and include aliphatic, alicyclic, and aromatic compounds. This category also includes some materials of complex composition which can be a source of a variety of hydrocarbons.

As a rule these compounds are chemically inert. Typical are petrolatum, paraffin, and mineral oil, all of which are widely used as emollients and vehicles in cosmetics because of their nonvolatility and unctuous character. Lower molecular weight volatile hydrocarbons are used as solvents (e.g., mineral spirits). The hydrocarbons which are normally gases at room temperature (e.g., butane or propane) are used as aerosol propellants. Finally, it should be noted that many synthetic polymers are hydrocarbons (e.g. polyethylene).

### With Emollient Properties

Emollient are cosmetic ingredients that help to maintain the soft, smooth, and pliable appearance of skin. Emollients function, via their ability to remain on the skin surface or in the stratum corneum, and act as lubricants to reduce flaking, and improve the skin's appearance.

C₁₂₋₂₀ isoparaffin, C₂₀₋₄₀ isoparaffin, C₉₋₁₆ isoparaffin, eicosane, hydrogenated polydecene, hydrogenated polydodecene, hydrogenated polyisobutene, isoeicosane, isohexadecane, mineral oil, octadecane, squalene.

### With Occlusive Properties

Occlusive are cosmetic ingredients that slow down the evaporation of water from the skin surface. This function is different from antiperspirant agents that interfere with the delivery of liquid water to the skin surface. By blocking the evaporative loss of water, occlusive materials increase the water content of skin. Occlusive agents are generally lipids that tend to remain on the skin surface

C₁₈₋₂₆ olefin, C₁₈₋₇₀ isoparaffin, C₂₀₋₂₄ olefin, C₂₄₋₂₈ olefin, C₂₄₋₃₀ olefin, C₂₆₋₅₄ olefin, C₂₈₋₃₆ olefin, C₃₀₋₄₅ Olefin, C₄₋₁₂ polyolefin, didecene, diethylhexylcyclohexane, mineral oil, paraffin, pentahydrosqualene, petrolatum, pristane, squalane.

With Solvent Properties

C₁₀₋₁₁ isoparaffin, C₁₀₋₁₂ isoparaffin, C₁₁₋₁₂ isoparaffin, C₁₁₋₁₃ isoparaffin, C₁₁₋₁₄ Isoparaffin, C₁₂₋₁₄ isoparaffin, C₁₃₋₁₄ isoparaffin, C₁₃₋₁₆ isoparaffin, decane, decene, dodecene, heptane, hexadecene, hexane, isododecane, limonene, octadecene, octene, petroleum distillates, tetradecene.

### Heterocyclic Compounds (Including Salts; Excluding Polymers and Imidazolines)

Heterocyclic compounds are aromatic or alicyclic compounds in which the ring contains one or more atoms other than carbon. The most common elements found in heterocyclics are nitrogen (e.g., PCA, zinc pyrithione, or pyridoxine), oxygen (e.g., furfural or dimethyl isosorbide), or sulfur (e.g., dibenzothiophene). Multiple replacement of carbon in rings is not uncommon, as in methylchloroisothiazolinone [N+S], guanosine [4N+O], or morpholine [N+O]; caprylyl pyrrolidone, lauryl pyrrolidone, myristyl nicotinate, pyridoxine dicaprylate, pyridoxine dilaurate, pyridoxine dioctenoate, pyridoxine dipalmitate.

The UV absorber Composition according to the present invention preferably comprises

1 to 80% of component (a),
10 to 90% b.w. of component (b), and
0 to 20% b.w. of component (c),
based on the total weight of the UV absorber composition.

The cosmetic or pharmaceutical compositions according to the present invention can be prepared by physically mixing the UV absorber(s) with the adjuvant using customary methods, for example by simply stirring together the individual components, especially by making use of the dissolution properties of already known cosmetic UV absorbers, like octyl methoxy cinnamate, salicylic acid isooctyl ester, etc. The UV absorber can be used, for example, without further treatment, or in the micronised state, or in the form of a powder.

Cosmetic or pharmaceutical preparations contain from 0.05-40% by weight, based on the total weight of the composition, of the UV absorber composition according to the present invention.

The cosmetic or pharmaceutical preparations may be, for example, creams, gels, lotions, alcoholic and aqueous/alcoholic solutions, emulsions, wax/fat compositions, stick preparations, powders or ointments. In addition to the above mentioned UV filters, the cosmetic or pharmaceutical preparations may contain further adjuvants as described below.

As water- and oil-containing emulsions (e.g. W/O, O/W, O/W/O and W/O/W emulsions or microemulsions) the preparations contain, for example, from 0.1 to 30% by weight, preferably from 0.1 to 15% by weight and especially from 0.5 to 10% by weight, based on the total weight of the composition, of one or more UV absorbers, from 1 to 60% by weight, especially from 5 to 50% by weight and preferably from 10 to 35% by weight, based on the total weight of the composition, of at least one oil component, from 0 to 30% by weight, especially from 1 to 30% by weight and preferably from 4 to 20% by weight, based on the total weight of the composition, of at least one emulsifier, from 10 to 90% by weight, especially from 30 to 90% by weight, based on the total weight of the composition, of water, and from 0 to 88.9% by weight, especially from 1 to 50% by weight, of further cosmetically acceptable adjuvants.

The cosmetic or pharmaceutical compositions/preparations according to the invention may also contain one or one more additional compounds like fatty alcohols, esters of fatty acids, natural or synthetic triglycerides including glyceryl esters and derivatives, pearlescent waxes: hydrocarbon oils: silicones or siloxanes, organosubstituted super-fatting agents, surfactants consistency regulators/thickeners and rheology modifiers, polymers, biogenic active ingredients, deodorising active ingredients, anti-dandruff agents, antioxidants, hydrotropic agents, preservatives and bacteria-inhibiting agents, perfume oils, colourants, polymeric beads or hollow spheres as spf enhancers.

### Cosmetic or Pharmaceutical Preparations

Cosmetic or pharmaceutical formulations are contained in a wide variety of cosmetic preparations. There come into consideration, for example, especially the following preparations:
skin-care preparations, e.g. skin-washing and cleansing preparations in the form of tablet-form or liquid soaps, soapless detergents or washing pastes,
bath preparations, e.g. liquid (foam baths, milks, shower preparations) or solid bath preparations, e.g. bath cubes and bath salts;
skin-care preparations, e.g. skin emulsions, multi-emulsions or skin oils;
cosmetic personal care preparations, e.g. facial make-up in the form of day creams or powder creams, face powder (loose or pressed), rouge or cream make-up, eye-care preparations, e.g. eyeshadow preparations, mascara, eyeliner, eye creams or eye-fix creams; lip-care preparations, e.g. lipsticks, lip gloss, lip contour pencils, nail-care preparations, such as nail varnish, nail varnish removers, nail hardeners or cuticle removers;
foot-care preparations, e.g. foot baths, foot powders, foot creams or foot balsams, special deodorants and antiperspirants or callus-removing preparations;
light-protective preparations, such as sun milks, lotions, creams or oils, sunblocks or tropicals, pre-tanning preparations or after-sun preparations;
skin-tanning preparations, e.g. self-tanning creams;
depigmenting preparations, e.g. preparations for bleaching the skin or skin-lightening preparations;
insect-repellents, e.g. insect-repellent oils, lotions, sprays or sticks;
deodorants, such as deodorant sprays, pump-action sprays, deodorant gels, sticks or roll-ons;
antiperspirants, e.g. antiperspirant sticks, creams or roll-ons;
preparations for cleansing and caring for blemished skin, e.g. synthetic detergents (solid or liquid), peeling or scrub preparations or peeling masks;
hair-removal preparations in chemical form (depilation), e.g. hair-removing powders, liquid hair-removing preparations, cream- or paste-form hair-removing preparations, hair-removing preparations in gel form or aerosol foams;
shaving preparations, e.g. shaving soap, foaming shaving creams, non-foaming shaving creams, foams and gels, preshave preparations for dry shaving, aftershaves or aftershave lotions;
fragrance preparations, e.g. fragrances (eau de Cologne, eau de toilette, eau de parfum, parfum de toilette, perfume), perfume oils or perfume creams;
cosmetic hair-treatment preparations, e.g. hair-washing preparations in the form of shampoos and conditioners, hair-care preparations, e.g. pretreatment preparations, hair tonics, styling creams, styling gels, pomades, hair rinses, treatment packs, intensive hair treatments, hair-structuring preparations, e.g. hair-waving preparations for permanent waves (hot wave, mild wave, cold wave), hair-straightening preparations, liquid hair-setting preparations, hair foams, hairsprays, bleaching preparations, e.g. hydrogen peroxide solutions, lightening shampoos, bleaching creams, bleaching powders, bleaching pastes or oils, temporary, semi-permanent or permanent hair colourants, preparations containing self-oxidising dyes, or natural hair colourants, such as henna or chamomile.

### Presentation Forms

The final formulations listed may exist in a wide variety of presentation forms, for example:
in the form of liquid preparations as a W/O, O/W, O/W/O, W/O/W or PIT emulsion and all kinds of microemulsions,
in the form of a gel,
in the form of an oil, a cream, milk or lotion,
in the form of a powder, a lacquer, a tablet or make-up,
in the form of a stick,
in the form of a spray (spray with propellent gas or pump-action spray) or an aerosol,
in the form of a foam, or
in the form of a paste.

Of special importance as cosmetic preparations for the skin are light-protective preparations, such as sun milks, lotions, creams, oils, sunblocks or tropicals, pretanning preparations or after-sun preparations, also skin-tanning preparations, for example self-tanning creams. Of particular interest are sun protection creams, sun protection lotions, sun protection milk and sun protection preparations in the form of a spray.

Of special importance as cosmetic preparations for the hair are the above-mentioned preparations for hair treatment, especially hair-washing preparations in the form of shampoos, hair conditioners, hair-care preparations, e.g. pretreatment preparations, hair tonics, styling creams, styling gels, pomades, hair rinses, treatment packs, intensive hair treatments, hair-straightening preparations, liquid hair-setting preparations, hair foams and hairsprays. Of special interest are hair-washing preparations in the form of shampoos.

A shampoo has, for example, the following composition: from 0.01 to 5% by weight of a UV absorber according to the invention, 12.0% by weight of sodium laureth-2-sulfate, 4.0% by weight of cocamidopropyl betaine, 3.0% by weight of sodium chloride, and water ad 100%.

Other typical ingredients in such formulations are preservatives, bactericides and bacteriostatic agents, perfumes, dyes, pigments, thickening agents, moisturizing agents, humectants, fats, oils, waxes or other typical ingredients of cosmetic and personal care formulations such as alcohols, poly-alcohols, polymers, electrolytes, organic solvents, silicon derivatives, emollients, emulsifiers or emulsifying surfactants, surfactants, dispersing agents, antioxidants, anti-irritants and anti-inflammatory agents etc.

The cosmetic preparation according to the invention is distinguished by excellent protection of human skin against the damaging effect of sunlight.

### EXAMPLES

### Formulation Examples

Example 1: Emulsion High Protection

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Ethylhexyl Methoxycinnnamate | 5.00 | 5.00 | 5.00 | 5.00 | 5.00 | 5.00 | 5.00 | 5.00 | 5.00 |
| | Cyclomethicone | 2.00 | 2.00 | 2.00 | 2.00 | 2.00 | 2.00 | 2.00 | 2.00 | 2.00 |
| | Octadecene/MA Copolymer | | 3.00 | | | | | | | |
| | (and) Methyl Acethyl Ricinoleate | | | | | | | | | |
| | (and) Di-methylhepthyl | | | | | | | | | |
| | Adipate | | | | | | | | | |
| | C30-38 olefin/isopropyl | | | 2.00 | | | | | | |
| | maleate/MA copolymer | | | | | | | | | |
| | Hydrogenated Dimer Dilinoleyl/ | | | | 3.00 | | | | | |
| | Dimethylcarbonate Co- | | | | | | | | | |
| | polymer | | | | | | | | | |
| | Cyclopentasiloxane (and) Acrylates/ | | | | | 7.00 | | | | |
| | Polytrimethylsiloxymethacrylate | | | | | | | | | |
| | Copolymer | | | | | | | | | |
| | Isododecane (and) Acrylates/ | | | | | | 8.00 | | | |
| | Polytrimethylsiloxymethacrylate | | | | | | | | | |
| | Copolymer | | | | | | | | | |
| | Poly(Glycol Adipate)/Bis- | | | | | | | 5.00 | | |
| | Hydroxyethyoxypropyl Dimethicone | | | | | | | | | |
| | Copolymer | | | | | | | | | |
| | Ethylhexyl Palmitate | 2.00 | 2.00 | 2.00 | 2.00 | 2.00 | 2.00 | 2.00 | 2.00 | 2.00 |
| | Ethylhexyl Triazone | 2.00 | 2.00 | 2.00 | 2.00 | 2.00 | 2.00 | 2.00 | 2.00 | 2.00 |
| | Pentaerythrityl Distearate | | 1.50 | | | | | 3.00 | | 5.00 |
| | Glyceryl Dibehenate (and) Tri- | | | 2.00 | | 4.00 | | | | |
| | behenin (and) Glyceryl Behenate | | | | | | | | | |
| | Glyceryl Stearate | 1.50 | | | 1.50 | | 1.50 | | 1.50 | |
| | C20-22 Alkyl Phosphate (and) | | 3.00 | | | | 2.50 | | | |
| | C20-22 Alcohols | | | | | | | | | |
| | Candelilla/Jojoba/Rice Bran | | | 6.00 | | | | 4.00 | | |
| | Polyglyceryl-3 esters (and) | | | | | | | | | |
| | glyceryl stearate (and) Cetearyl | | | | | | | | | |
| | alcohol (and) Sodium | | | | | | | | | |
| | stearoyl lactylate | | | | | | | | | |
| | Glyceryl oleate citrate (and) | | | | 5.00 | | | | 6.00 | |
| | Caprylic/Capric Triglycerides | | | | | | | | | |
| | Potassium Cetyl Phosphate | 1.80 | | | | 1.80 | | | | 1.80 |
| | VP/Eicosene Copolymer | 0.60 | | | | 0.60 | | | | |
| | Dimethyl capramide | 2.00 | 4.00 | 6.00 | 8.00 | 9.00 | 12.00 | 14.00 | 16.00 | 10.00 |
| | Bis-Ethylhexyloxyphenol | 1.00 | 2.00 | 3.00 | 4.00 | 5.00 | 6.00 | 7.00 | 2.00 | 4.00 |
| | Methoxyphenyl Triazine (Tinosorb | | | | | | | | | |
| | S) | | | | | | | | | |
| Part B | Aqua | Qs | Qs | Qs | Qs | Qs to 100 | Qs to 100 | Qs to 100 | Qs to 100 | Qs to 100 |
| | | to 100 | to 100 | to 100 | to 100 | | | | | |
| | Methylene Bis-Benzotriazolyl | 2.00 | 2.00 | 2.00 | 2.00 | 2.00 | 2.00 | 2.00 | 2.00 | 2.00 |
| | Tetramethylbutylphenol (and) | | | | | | | | | |
| | Aqua (and) Decyl Glucoside | | | | | | | | | |
| | (and) Propylene Glycol (and) | | | | | | | | | |
| | Xanthan Gum | | | | | | | | | |
| | PVP/dimethylconylacrylate/polycarbamyl/ | | | | | | | | | 5.00 |
| | polyglycol ester | | | | | | | | | |
| | Polyester-5 | | | | | | | | 3.00 | |
| | Acrylates/Palmeth-25 Acrylate | 0.25 | | | | | | | | 0.25 |
| | Copolymer | | | | | | | | | |
| | Disteareth-75 IPDI | | 0.25 | 3.00 | | | | | | |
| | Disteareth-100 IPDI | | | | 0.25 | 3.00 | | | | |
| | Acrylates/Vinyl Neodecanoate | | | | | | 1.50 | 5.00 | 8.00 | |
| | Crosspolymer | | | | | | | | | |
| | Glycerin | 2.00 | 2.00 | 2.00 | 2.00 | 2.00 | 2.00 | 2.00 | 2.00 | 2.00 |
| | Disodium EDTA | 0.20 | 0.20 | 0.20 | 0.20 | 0.20 | 0.20 | 0.20 | 0.20 | 0.20 |
| Part C | Phenoxyethanol (and) Methyl- | 0.70 | 0.70 | 0.70 | 0.70 | 0.70 | 0.70 | 0.70 | 0.70 | 0.70 |
| | paraben (and) Ethylparaben | | | | | | | | | |
| | (and) Butylparaben (and) Propylparaben | | | | | | | | | |
| | (and) Isobutylparaben | | | | | | | | | |
| | Tocopheryl Acetate | 0.30 | 0.30 | 0.30 | 0.30 | 0.30 | 0.30 | 0.30 | 0.30 | 0.30 |

Example 2: Sun Milk

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | C12-15 Alkyl Benzoate | 4.00 | 4.00 | 4.00 | 4.00 | 4.00 | 4.00 | 4.00 | 4.00 | 4.00 |
| | Butyl Methoxydibenzoyl- | 1.00 | 1.00 | 1.00 | 1.00 | 1.00 | 1.00 | 1.00 | 1.00 | 1.00 |
| | methane | | | | | | | | | |
| | Octocrylene | 7.50 | 7.50 | 7.50 | 7.50 | 7.50 | 7.50 | 7.50 | 7.50 | 7.50 |
| | Cyclopentasiloxane | 2.00 | 2.00 | 2.00 | 2.00 | 2.00 | 2.00 | 2.00 | 2.00 | 2.00 |
| | Terephthalylidene Dicamphor | 0.30 | 0.30 | 0.30 | 0.30 | 0.30 | 0.30 | 0.30 | 0.30 | 0.30 |
| | Sulfonic Acid | | | | | | | | | |
| | Titanium Dioxide particles size | 1.70 | 1.70 | 1.70 | 1.70 | 1.70 | 1.70 | 1.70 | 1.70 | 1.70 |
| | 10 to 100 nm | | | | | | | | | |
| | Pentaerythrityl Distearate | | 1.50 | | | | | 1.00 | | 2.00 |
| | Glyceryl Dibehenate (and) | | | 2.00 | | 4.00 | | | | |
| | Tribehenin (and) Glyceryl | | | | | | | | | |
| | Behenate | | | | | | | | | |
| | Stearic Acid | 0.50 | 0.50 | 0.50 | 0.50 | 0.50 | 0.50 | 0.50 | 0.50 | 0.50 |
| | PEG-100 Stearate (and) Glyceryl | 0.80 | 0.80 | 0.80 | 0.80 | 0.80 | 0.80 | 0.80 | 0.80 | 0.80 |
| | Stearate | | | | | | | | | |
| | Octadecene/MA Copolymer | | 3.00 | | | | | | | |
| | (and) Methyl Acethyl | | | | | | | | | |
| | Ricinoleate | | | | | | | | | |
| | (and) Di-methylhepthyl | | | | | | | | | |
| | Adipate | | | | | | | | | |
| | C30-38 olefin/isopropyl | | | 2.00 | | | | | | |
| | maleate/MA copolymer | | | | | | | | | |
| | Hydrogenated Dimer Dilinoleyl/ | | | | 3.00 | | | | | |
| | Dimethylcarbonate Co- | | | | | | | | | |
| | polymer | | | | | | | | | |
| | Cyclopentasiloxane (and) | | | | | 7.00 | | | | |
| | Acrylates/Polytrimethylsiloxy- | | | | | | | | | |
| | methacrylate Copolymer | | | | | | | | | |
| | Isododecane (and) Acrylates/ | | | | | | 8.00 | | | |
| | Polytrimethylsiloxymethacrylate | | | | | | | | | |
| | Copolymer | | | | | | | | | |
| | Poly(Glycol Adipate)/Bis- | | | | | | | 5.00 | | |
| | Hydroxyethyoxypropyl | | | | | | | | | |
| | Dimethicone | | | | | | | | | |
| | Copolymer | | | | | | | | | |
| | C20-22 Alkyl Phosphate (and) | | 2.50 | | | | 3.00 | | | |
| | C20-22 Alcohols | | | | | | | | | |
| | Candelilla/Jojoba/Rice Bran | | | 5.00 | | | | 6.00 | | |
| | Polyglyceryl-3 esters (and) | | | | | | | | | |
| | glyceryl stearate (and) Cetearyl | | | | | | | | | |
| | alcohol (and) Sodium | | | | | | | | | |
| | stearoyl lactylate | | | | | | | | | |
| | Glyceryl oleate citrate (and) | | | | 4.00 | | | | 5.00 | |
| | Caprylic/Capric Triglycerides | | | | | | | | | |
| | Potassium Cetyl Phosphate | 1.30 | | | | 1.30 | | | | 1.30 |
| | PVP/Eicosene Copolymer | 1.00 | | | | | | | | |
| | Dimethyl capramide | 2.00 | 4.00 | 6.00 | 8.00 | 9.00 | 12.00 | 14.00 | 16.00 | 10.00 |
| | Bis-Ethylhexyloxyphenol | 1.00 | 2.00 | 3.00 | 4.00 | 5.00 | 6.00 | 7.00 | 2.00 | 4.00 |
| | Methoxyphenyl | | | | | | | | | |
| | Triazine (Tinosorb | | | | | | | | | |
| | S) | | | | | | | | | |
| Part B | Aqua | Qs to 100 | Qs to 100 | Qs to 100 | Qs to 100 | Qs to 100 | Qs to 100 | Qs to 100 | Qs to 100 | Qs to 100 |
| | Glycerin | 2.00 | 2.00 | 2.00 | 2.00 | 2.00 | 2.00 | 2.00 | 2.00 | 2.00 |
| | Xanthan Gum | 0.20 | 0.20 | 0.20 | 0.20 | 0.20 | 0.20 | 0.20 | 0.20 | 0.20 |
| | PVP/dimethylconylacrylate/ | | | | | | | | | 6.00 |
| | polycarbamyl/ | | | | | | | | | |
| | polyglycol ester | | | | | | | | | |
| | Polyester-5 | | | | | | | | 3.00 | |
| | Disteareth-75 IPDI | | 0.25 | 3.00 | | | | | | |
| | Disteareth-100 IPDI | | | | 0.25 | 3.00 | | | | |
| | Acrylates/Vinyl Neodecanoate | | | | | | 1.50 | 5.00 | 8.00 | |
| | Crosspolymer | | | | | | | | | |
| | Acrylates/C10-30 Alkyl Acrylate | 0.15 | | | | | | | | 0.15 |
| | Crosspolymer | | | | | | | | | |
| | Disodium EDTA | 0.15 | 0.15 | 0.15 | 0.15 | 0.15 | 0.15 | 0.15 | 0.15 | 0.15 |
| Part C | Triethanolamine | Qs | Qs | Qs | Qs | Qs | Qs | Qs | Qs | Qs |
| | Dimethicone | 0.40 | 0.40 | 0.40 | 0.40 | 0.40 | 0.40 | 0.40 | 0.40 | 0.40 |
| | Phenoxyethanol (and) Methyl- | 0.70 | 0.70 | 0.70 | 0.70 | 0.70 | 0.70 | 0.70 | 0.70 | 0.70 |
| | paraben (and) Ethylparaben | | | | | | | | | |
| | (and) Butylparaben (and) | | | | | | | | | |
| | Propylparaben | | | | | | | | | |
| | (and) Isobutylparaben | | | | | | | | | |
| | Tocopheryl Acetate | 0.90 | 0.90 | 0.90 | 0.90 | 0.90 | 0.90 | 0.90 | 0.90 | 0.90 |

Example 3: Sun Milk

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Butylene Glycol Dicaprylate/ | 5.00 | 5.00 | 5.00 | 5.00 | 5.00 | 5.00 | 5.00 | 5.00 | 5.00 |
| | Dicaprate | | | | | | | | | |
| | Butyl | 4.50 | 4.50 | 4.50 | 4.50 | 4.50 | 4.50 | 4.50 | 4.50 | 4.50 |
| | Methoxydibenzoylmethane | | | | | | | | | |
| | Octocrylene | 3.50 | 3.50 | 3.50 | 3.50 | 3.50 | 3.50 | 3.50 | 3.50 | 3.50 |
| | Titanium Dioxide particles size | 3.00 | 3.00 | 3.00 | 3.00 | 3.00 | 3.00 | 3.00 | 3.00 | 3.00 |
| | 10 to 100 nm | | | | | | | | | |
| | Ethylhexyl Methoxycinnamate | 2.00 | 2.00 | 2.00 | 2.00 | 2.00 | 2.00 | 2.00 | 2.00 | 2.00 |
| | Octadecene/MA Copolymer | | 3.00 | | | | | | | |
| | (and) Methyl Acethyl | | | | | | | | | |
| | Ricinoleate | | | | | | | | | |
| | (and) Di-methylhepthyl | | | | | | | | | |
| | Adipate | | | | | | | | | |
| | C30-38 olefin/isopropyl | | | 2.00 | | | | | | |
| | maleate/MA copolymer | | | | | | | | | |
| | Hydrogenated Dimer Dilinoleyl/ | | | | 3.00 | | | | | |
| | Dimethylcarbonate Co- | | | | | | | | | |
| | polymer | | | | | | | | | |
| | Cyclopentasiloxane (and) | | | | | 7.00 | | | | |
| | Acrylates/ | | | | | | | | | |
| | Polytrimethylsiloxymethacrylate | | | | | | | | | |
| | Copolymer | | | | | | | | | |
| | Isododecane (and) Acrylates/ | | | | | | 8.00 | | | |
| | Polytrimethylsiloxymethacrylate | | | | | | | | | |
| | Copolymer | | | | | | | | | |
| | Poly(Glycol Adipate)/Bis- | | | | | | | 5.00 | | |
| | Hydroxyethyoxypropyl | | | | | | | | | |
| | Dimethicone | | | | | | | | | |
| | Copolymer | | | | | | | | | |
| | Dicaprylyl Carbonate | 2.00 | 2.00 | 2.00 | 2.00 | 2.00 | 2.00 | 2.00 | 2.00 | 2.00 |
| | C20-22 Alkyl Phosphate (and) | | 6.00 | | | | 6.00 | | | |
| | C20-22 Alcohols | | | | | | | | | |
| | Candelilla/Jojoba/Rice Bran | | | 5.00 | | | | 5.00 | | |
| | Polyglyceryl-3 esters (and) | | | | | | | | | |
| | glyceryl | | | | | | | | | |
| | stearate (and) Cetearyl | | | | | | | | | |
| | alcohol (and) Sodium stearoyl | | | | | | | | | |
| | lactylate | | | | | | | | | |
| | Glyceryl oleate citrate (and) | | | | 6.00 | | | | 6.00 | |
| | Caprylic/Capric Triglycerides | | | | | | | | | |
| | Glyceryl Stearate Citrate | 2.00 | | | | 2.00 | | | | 2.00 |
| | Glyceryl Dibehenate (and) Tri- | | | 2.00 | | 4.00 | | | | |
| | behenin (and) Glyceryl Behenate | | | | | | | | | |
| | Pentaerythrityl Distearate | | 1.50 | | | | | 3.00 | | |
| | Stearyl Alcohol | 1.80 | 0.50 | | 1.80 | | 1.80 | | 1.80 | |
| | Hydrogenated Coco-Glycerides | 1.60 | 1.20 | 1.60 | 1.60 | 1.60 | 1.60 | 1.00 | 1.60 | 0.80 |
| | PVP/Hexadecene Copolymer | 0.50 | | | | | | | | 0.50 |
| | Dimethyl capramide | 2.00 | 4.00 | 6.00 | 8.00 | 9.00 | 12.00 | 14.00 | 16.00 | 10.00 |
| | Bis-Ethylhexyloxyphenol | 1.00 | 2.00 | 3.00 | 4.00 | 5.00 | 6.00 | 7.00 | 2.00 | 4.00 |
| | Methoxyphenyl | | | | | | | | | |
| | Triazine (Tinosorb | | | | | | | | | |
| | S) | | | | | | | | | |
| Part B | Aqua | Qs to 100 | Qs to 100 | Qs to 100 | Qs to 100 | Qs to 100 | Qs to 100 | Qs to 100 | Qs to 100 | Qs to 100 |
| | PVP/dimethylconylacrylate/ | | | | | | | | | 6.00 |
| | polycarbamyl/ | | | | | | | | | |
| | polyglycol ester | | | | | | | | | |
| | Polyester-5 | | | | | | | | 3.00 | |
| | Xanthan Gum | 0.25 | 0.25 | 0.25 | 0.25 | 0.25 | 0.25 | 0.25 | 0.25 | 0.25 |
| | Disteareth-75 IPDI | | 0.25 | 3.00 | | | | | | |
| | Disteareth-100 IPDI | | | | 0.25 | 3.00 | | | | |
| | Acrylates/Vinyl Neodecanoate | | | | | | 1.50 | 5.00 | 8.00 | |
| | Crosspolymer | | | | | | | | | |
| | Acrylates/C10-30 Alkyl Acrylate | 0.15 | | | | | | | | 0.15 |
| | Crosspolymer | | | | | | | | | |
| | Trisodium EDTA | 0.20 | 0.20 | 0.20 | 0.20 | 0.20 | 0.20 | 0.20 | 0.20 | 0.20 |
| Part C | Alcohol Denatured | 2.00 | 2.00 | 2.00 | 2.00 | 2.00 | 2.00 | 2.00 | 2.00 | 2.00 |
| Part D | Sodium Hydroxide | Qs | Qs | Qs | Qs | Qs | Qs | Qs | Qs | Qs |
| | Phenoxyethanol (and) Methyl- | 0.70 | 0.70 | 0.70 | 0.70 | 0.70 | 0.70 | 0.70 | 0.70 | 0.70 |
| | paraben (and) Ethylparaben | | | | | | | | | |
| | (and) Butylparaben (and) | | | | | | | | | |
| | Propylparaben | | | | | | | | | |
| | (and) Isobutylparaben | | | | | | | | | |
| | Tocopheryl Acetate | 1.00 | 1.00 | 1.00 | 1.00 | 1.00 | 1.00 | 1.00 | 1.00 | 1.00 |

Example 4: Emulsion High Protection

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Ethylhexyl Methoxycinnnamate | 9.80 | 9.80 | 9.80 | 9.80 | 9.80 | 9.80 | 9.80 | 9.80 | 9.80 |
| | Cyclomethicone | 1.00 | 1.00 | 1.00 | 1.00 | 1.00 | 1.00 | 1.00 | 1.00 | 1.00 |
| | Neopentyl Glycol Diheptanoate | 6.00 | | 5.00 | | | | | | |
| | Propylene Glycol Dibenzoate | 4.00 | | | | | | | | |
| | Polyester-7 and Neopenthyl | | | | | 8.00 | | | | |
| | Glycol Diheptanoate | | | | | | | | | |
| | Diethylhexyl Syringylidene- | | 6.00 | | | | | | | |
| | malonate | | | | | | | | | |
| | Polyester 8 | | | 4.00 | | | | | | |
| | Diethylhexyl Malate | | | | 7.00 | | | | | |
| | PPG-3 Myristyl Ether | | | | | | 6.00 | | | |
| | Neoheptanoate | | | | | | | | | |
| | Phenethyl Benzoate | | | | | | | 8.00 | | |
| | Isopropyl PPG-2 Isodeceth-7 | | | | | | | | 4.00 | |
| | Carboxylate | | | | | | | | | |
| | Isopropyl C12-15-Pareth-9 | | | | | | | | 4.00 | |
| | Carboxylate | | | | | | | | | |
| | Cetyl PPG-2 Isodeceth-7 | | | | | | | | 4.00 | |
| | Carboxylate | | | | | | | | | |
| | Ethylhexyl Palmitate | 3.00 | 3.00 | 3.00 | 3.00 | 3.00 | 3.00 | 3.00 | 3.00 | 3.00 |
| | Ethylhexyl Triazone | 2.00 | 2.00 | 2.00 | 2.00 | 2.00 | 2.00 | 2.00 | 2.00 | 2.00 |
| | Glyceryl Stearate | 1.50 | 1.50 | 1.50 | 1.50 | 1.50 | 1.50 | 1.50 | 1.50 | 1.50 |
| | Potassium Cetyl Phosphate | 1.80 | 1.80 | 1.80 | 1.80 | 1.80 | 1.80 | 1.80 | 1.80 | 1.80 |
| | VP/Eicosene Copolymer | 0.60 | 0.60 | 0.60 | 0.60 | 0.60 | 0.60 | 0.60 | 0.60 | 0.60 |
| | Dimethyl capramide | 2.00 | 4.00 | 6.00 | 8.00 | 9.00 | 12.0 | 14.0 | 16.0 | 10.0 |
| | Bis-Ethylhexyloxyphenol | 1.00 | 2.00 | 3.00 | 4.00 | 5.00 | 6.00 | 7.00 | 2.00 | 4.00 |
| | Methoxyphenyl | | | | | | | | | |
| | Triazine (Tinosorb S) | | | | | | | | | |
| Part B | Aqua | Qs to 100 | Qs to 100 | Qs to 100 | Qs to 100 | Qs to 100 | Qs to 100 | Qs to 100 | Qs to 100 | Qs to 100 |
| | Methylene Bis-Benzotriazolyl | 2.00 | 2.00 | 2.00 | 2.00 | 2.00 | 2.00 | 2.00 | 2.00 | 2.00 |
| | Tetramethylbutylphenol (and) | | | | | | | | | |
| | Aqua (and) Decyl Glucoside | | | | | | | | | |
| | (and) Propylene Glycol (and) | | | | | | | | | |
| | Xanthan Gum | | | | | | | | | |
| | Acrylates/Palmeth-25 Acrylate | 0.25 | 0.25 | 0.25 | 0.25 | 0.25 | 0.25 | 0.25 | 0.25 | 0.25 |
| | Copolymer | | | | | | | | | |
| | Glycerin | 2.00 | 2.00 | 2.00 | 2.00 | 2.00 | 2.00 | 2.00 | 2.00 | 2.00 |
| | Disodium EDTA | 0.20 | 0.20 | 0.20 | 0.20 | 0.20 | 0.20 | 0.20 | 0.20 | 0.20 |
| Part C | Phenoxyethanol (and) Methyl- | 0.70 | 0.70 | 0.70 | 0.70 | 0.70 | 0.70 | 0.70 | 0.70 | 0.70 |
| | paraben (and) Ethylparaben | | | | | | | | | |
| | (and) Butylparaben (and) | | | | | | | | | |
| | Propylparaben | | | | | | | | | |
| | (and) Isobutylparaben | | | | | | | | | |
| | Tocopheryl Acetate | 0.30 | 0.30 | 0.30 | 0.30 | 0.30 | 0.30 | 0.30 | 0.30 | 0.30 |

Example 5: Milk Very Hihgh Protection

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Ethylhexyl Methoxycinnnamate | 10.00 | 10.00 | 10.00 | 10.00 | 10.00 | 10.00 | 10.00 | 10.00 | 10.00 |
| | Cyclomethicone | 1.00 | 1.00 | 1.00 | 1.00 | 1.00 | 1.00 | 1.00 | 1.00 | 1.00 |
| | Titanium Dioxide particles size | 0.32 | 0.32 | 0.32 | 0.32 | 0.32 | 0.32 | 0.32 | 0.32 | 0.32 |
| | 10 to 100 nm | | | | | | | | | |
| | Neopentyl Glycol Diheptanoate | 6.00 | | 5.00 | | | | | | |
| | Propylene Glycol Dibenzoate | 4.00 | | | | | | | | |
| | Polyester-7 and Neopenthyl | | | | | 8.00 | | | | |
| | Glycol Diheptanoate | | | | | | | | | |
| | Diethylhexyl Syringylidene- | | 6.00 | | | | | | | |
| | malonate | | | | | | | | | |
| | Polyester 8 | | | 4.00 | | | | | | |
| | Diethylhexyl Malate | | | | 7.00 | | | | | |
| | PPG-3 Myristyl Ether | | | | | | 6.00 | | | |
| | Neoheptanoate | | | | | | | | | |
| | Phenethyl Benzoate | | | | | | | 8.00 | | |
| | Isopropyl PPG-2 Isodeceth-7 | | | | | | | | 4.00 | |
| | Carboxylate | | | | | | | | | |
| | Isopropyl C12-15-Pareth-9 | | | | | | | | 4.00 | |
| | Carboxylate | | | | | | | | | |
| | Cetyl PPG-2 Isodeceth-7 | | | | | | | | 4.00 | |
| | Carboxylate | | | | | | | | | |
| | Isodecyl Neopentanoate | 3.50 | 3.50 | 3.50 | 3.50 | 3.50 | 3.50 | 3.50 | 3.50 | 3.50 |
| | Ethylhexyl Triazone | 4.50 | 4.50 | 4.50 | 4.50 | 4.50 | 4.50 | 4.50 | 4.50 | 4.50 |
| | Glyceryl Stearate | 2.50 | 2.50 | 2.50 | 2.50 | 2.50 | 2.50 | 2.50 | 2.50 | 2.50 |
| | Potassium Cetyl Phosphate | 1.80 | 1.80 | 1.80 | 1.80 | 1.80 | 1.80 | 1.80 | 1.80 | 1.80 |
| | Stearyl Alcohol | 1.00 | 1.00 | 1.00 | 1.00 | 1.00 | 1.00 | 1.00 | 1.00 | 1.00 |
| | Zinc Oxide (and) Trimethoxy- | 0.21 | 0.21 | 0.21 | 0.21 | 0.21 | 0.21 | 0.21 | 0.21 | 0.21 |
| | caprylylsilane | | | | | | | | | |
| | VP/Eicosene Copolymer | 0.60 | 0.60 | 0.60 | 0.60 | 0.60 | 0.60 | 0.60 | 0.60 | 0.60 |
| | Dimethyl capramide | 2.00 | 4.00 | 6.00 | 8.00 | 9.00 | 12.00 | 14.00 | 16.00 | 10.00 |
| | Bis-Ethylhexyloxyphenol | 1.00 | 2.00 | 3.00 | 4.00 | 5.00 | 6.00 | 7.00 | 2.00 | 4.00 |
| | Methoxyphenyl | | | | | | | | | |
| | Triazine (Tinosorb | | | | | | | | | |
| | S) | | | | | | | | | |
| Part B | Aqua | Qs to 100 | Qs to 100 | Qs to 100 | Qs to 100 | Qs to 100 | Qs to 100 | Qs to 100 | Qs to 100 | Qs to 100 |
| | Methylene Bis-Benzotriazolyl | 3.10 | 3.10 | 3.10 | 3.10 | 3.10 | 3.10 | 3.10 | 3.10 | 3.10 |
| | Tetramethylbutylphenol | | | | | | | | | |
| | (and) Aqua | | | | | | | | | |
| | (and) Decyl Glucoside (and) | | | | | | | | | |
| | Propylene | | | | | | | | | |
| | Glycol (and) Xanthan | | | | | | | | | |
| | Gum | | | | | | | | | |
| | Glycerin | 2.00 | 2.00 | 2.00 | 2.00 | 2.00 | 2.00 | 2.00 | 2.00 | 2.00 |
| | Disodium EDTA | 0.30 | 0.30 | 0.30 | 0.30 | 0.30 | 0.30 | 0.30 | 0.30 | 0.30 |
| Part C | Dimethicone | 0.40 | 0.40 | 0.40 | 0.40 | 0.40 | 0.40 | 0.40 | 0.40 | 0.40 |
| | Phenoxyethanol (and) Methyl- | 0.70 | 0.70 | 0.70 | 0.70 | 0.70 | 0.70 | 0.70 | 0.70 | 0.70 |
| | paraben (and) Ethylparaben | | | | | | | | | |
| | (and) Butylparaben (and) | | | | | | | | | |
| | Propylparaben | | | | | | | | | |
| | (and) Isobutyl- | | | | | | | | | |
| | paraben | | | | | | | | | |
| | Tocopheryl Acetate | 0.30 | 0.30 | 0.30 | 0.30 | 0.30 | 0.30 | 0.30 | 0.30 | 0.30 |

Example 6: Emulsion High Protection

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Ethylhexyl Methoxycinnnamate | 9.80 | 9.80 | 9.80 | 9.80 | 9.80 | 9.80 | 9.80 | 9.80 | 9.80 |
| | Cyclomethicone | 2.00 | 2.00 | 2.00 | 2.00 | 2.00 | 2.00 | 2.00 | 2.00 | 2.00 |
| | Ethylhexyl Palmitate | 2.00 | 2.00 | 2.00 | 2.00 | 2.00 | 2.00 | 2.00 | 2.00 | 2.00 |
| | Ethylhexyl Triazone | 2.00 | 2.00 | 2.00 | 2.00 | 2.00 | 2.00 | 2.00 | 2.00 | 2.00 |
| | Glyceryl Stearate | 1.50 | 1.50 | 1.50 | 1.50 | 1.50 | 1.50 | 1.50 | 1.50 | 1.50 |
| | Butylene Glycol Cocoate | 4.00 | 6.00 | 4.00 | 3.00 | 2.00 | 1.00 | 2.50 | 2.00 | 4.00 |
| | Potassium Cetyl Phosphate | 1.80 | 1.80 | 1.80 | 1.80 | 1.80 | 1.80 | 1.80 | 1.80 | 1.80 |
| | VP/Eicosene Copolymer | 0.60 | 0.60 | 0.60 | 0.60 | 0.60 | 0.60 | 0.60 | 0.60 | 0.60 |
| | Dimethyl capramide | 2.00 | 4.00 | 6.00 | 8.00 | 9.00 | 12.00 | 14.00 | 16.00 | 10.00 |
| | Bis-Ethylhexyloxyphenol | 1.00 | 2.00 | 3.00 | 4.00 | 5.00 | 6.00 | 7.00 | 2.00 | 4.00 |
| | Methoxyphenyl | | | | | | | | | |
| | Triazine (Tinosorb S) | | | | | | | | | |
| Part B | Aqua | Qs to 100 | Qs to 100 | Qs to 100 | Qs to 100 | Qs to 100 | Qs to 100 | Qs to 100 | Qs to 100 | Qs to 100 |
| | Methylene Bis-Benzotriazolyl | 2.00 | 2.00 | 2.00 | 2.00 | 2.00 | 2.00 | 2.00 | 2.00 | 2.00 |
| | Tetramethylbutylphenol (and) | | | | | | | | | |
| | Aqua (and) Decyl Glucoside | | | | | | | | | |
| | (and) Propylene Glycol (and) | | | | | | | | | |
| | Xanthan Gum | | | | | | | | | |
| | Acrylates/Palmeth-25 Acrylate | 0.25 | 0.25 | 0.25 | 0.25 | 0.25 | 0.25 | 0.25 | 0.25 | 0.25 |
| | Copolymer | | | | | | | | | |
| | Glycerin | 2.00 | 2.00 | 2.00 | 2.00 | 2.00 | 2.00 | 2.00 | 2.00 | 2.00 |
| | Disodium EDTA | 0.20 | 0.20 | 0.20 | 0.20 | 0.20 | 0.20 | 0.20 | 0.20 | 0.20 |
| Part C | Phenoxyethanol (and) Methyl- | 0.70 | 0.70 | 0.70 | 0.70 | 0.70 | 0.70 | 0.70 | 0.70 | 0.70 |
| | paraben (and) Ethylparaben | | | | | | | | | |
| | (and) Butylparaben (and) | | | | | | | | | |
| | Propylparaben | | | | | | | | | |
| | (and) Isobutylparaben | | | | | | | | | |
| | Styrene/Acrylates Copolymer | 5.00 | | | | | 3.00 | | 1.00 | |
| | PEG-6 Isostearate (and) | | 3.00 | | | | 1.00 | 4.00 | | 1.00 |
| | Hesperetin | | | | | | | | | |
| | Laurate | | | | | | | | | |
| | Polyacrylate-15 (and) Poly- | | | 4.00 | | | | | 2.00 | |
| | acrylate-17 | | | | | | | | | |
| | Carthamus Tinctorius | | | | 5.00 | | | | | 2.00 |
| | (Safflower) | | | | | | | | | |
| | Oleosomes | | | | | | | | | |
| | PTFE | | | | | 4.00 | | | | 2.00 |
| | Tocopheryl Acetate | 0.30 | 0.30 | 0.30 | 0.30 | 0.30 | 0.30 | 0.30 | 0.30 | 0.30 |

Example 7: Sun Cream

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Cetearyl glucoside | 4.00 | | | 4.00 | | | 4.00 | | |
| | Dicaprylyl Carbonate | 8.00 | 8.00 | 8.00 | 8.00 | 8.00 | 8.00 | 8.00 | 8.00 | 8.00 |
| | Octocrylene | 10.00 | 10.00 | 10.00 | 10.00 | 10.00 | 10.00 | 10.00 | 10.00 | 10.00 |
| | Butyl Methoxydibenzoyl- | 4.00 | 4.00 | 4.00 | 4.00 | 4.00 | 4.00 | 4.00 | 4.00 | 4.00 |
| | methane | | | | | | | | | |
| | Ethylhexyl salicyate | 3.00 | 3.00 | 3.00 | 3.00 | 3.00 | 3.00 | 3.00 | 3.00 | 3.00 |
| | Octadecene/MA Copolymer | | | | 3.00 | | | | | |
| | (and) Methyl Acethyl | | | | | | | | | |
| | Ricinoleate | | | | | | | | | |
| | (and) Di-methylhepthyl | | | | | | | | | |
| | Adipate | | | | | | | | | |
| | C30-38 olefin/isopropyl | | | | | 2.00 | | | | |
| | maleate/MA copolymer | | | | | | | | | |
| | Hydrogenated Dimer Dilinoleyl/ | | | | | | 3.00 | | | |
| | Dimethylcarbonate Co- | | | | | | | | | |
| | polymer | | | | | | | | | |
| | Cyclopentasiloxane (and) | | | | | | | 7.00 | | |
| | Acrylates/ | | | | | | | | | |
| | Polytrimethylsiloxymethacrylate | | | | | | | | | |
| | Copolymer | | | | | | | | | |
| | Isododecane (and) Acrylates/ | | | | | | | | 8.00 | |
| | Polytrimethylsiloxymethacrylate | | | | | | | | | |
| | Copolymer | | | | | | | | | |
| | Poly(Glycol Adipate)/Bis- | | | | | | | | | 5.00 |
| | Hydroxyethyoxypropyl | | | | | | | | | |
| | Dimethicone | | | | | | | | | |
| | Copolymer | | | | | | | | | |
| | Pentaerythrityl Distearate | | 1.50 | | | | | 3.00 | | 5.00 |
| | Glyceryl Dibehenate (and) Tri- | | | 2.00 | | 4.00 | | | | |
| | behenin (and) Glyceryl Behenate | | | | | | | | | |
| | Ethylhexyl Methoxycinnamate | 1.80 | 1.80 | 1.80 | 1.80 | 1.80 | 1.80 | 1.80 | 1.80 | 1.80 |
| | Sorbitan stearate (and) Sorbitol | | 5.00 | | | 5.00 | | | 5.00 | |
| | monolaurate | | | | | | | | | |
| | Sucrose polystearate | | | 5.00 | | | 5.00 | | | 5.00 |
| | Dimethyl capramide | 2.00 | 4.00 | 6.00 | 8.00 | 9.00 | 12.00 | 14.00 | 16.00 | 10.00 |
| | Bis-Ethylhexyloxyphenol | 1.00 | 2.00 | 3.00 | 4.00 | 5.00 | 6.00 | 7.00 | 2.00 | 4.00 |
| | Methoxyphenyl | | | | | | | | | |
| | Triazine (Tinosorb S) | | | | | | | | | |
| Part B | Water | Qs to 100 | Qs to 100 | Qs to 100 | Qs to 100 | Qs to 100 | Qs to 100 | Qs to 100 | Qs to 100 | Qs to 100 |
| | Disodium EDTA | 0.20 | 0.20 | 0.20 | 0.20 | 0.20 | 0.20 | 0.20 | 0.20 | 0.20 |
| | Glycerin | 2.00 | 2.00 | 2.00 | 2.00 | 2.00 | 2.00 | 2.00 | 2.00 | 2.00 |
| | Disteareth-75 IPDI | | 0.25 | 3.00 | | | | | | |
| | Disteareth-100 IPDI | | | | 0.25 | 3.00 | | | | |
| | Acrylates/Vinyl Neodecanoate | | | | | | 1.50 | 5.00 | 8.00 | |
| | Crosspolymer | | | | | | | | | |
| | PVP/dimethylconylacrylate/ | 3.00 | | | | | | | | |
| | polycarbamyl/ | | | | | | | | | |
| | polyglycol ester | | | | | | | | | |
| | Polyester-5 | | 5.00 | | | | | | | |
| | Sodium polyacrylate | 0.70 | 0.70 | 0.70 | 0.70 | 0.70 | | | | 0.70 |
| Part C | Dimethicone | 3.00 | 3.00 | 3.00 | 3.00 | 3.00 | 3.00 | 3.00 | 3.00 | 3.00 |
| | Corn Starch modified | 3.00 | 3.00 | 3.00 | 3.00 | 3.00 | 3.00 | 3.00 | 3.00 | 3.00 |
| Part D | Methylene Bis-Benzotriazolyl | 4.00 | 4.00 | 4.00 | 4.00 | 4.00 | 4.00 | 4.00 | 4.00 | 4.00 |
| | Tetramethylbutylphenol (and) | | | | | | | | | |
| | Aqua (and) Decyl Glucoside | | | | | | | | | |
| | (and) Propylene Glycol (and) | | | | | | | | | |
| | Xanthan Gum | | | | | | | | | |
| Part E | Phenoxyethanol (and) Methyl- | 1.00 | 1.00 | 1.00 | 1.00 | 1.00 | 1.00 | 1.00 | 1.00 | 1.00 |
| | paraben (and) Ethylparaben | | | | | | | | | |
| | (and) Butylparaben (and) | | | | | | | | | |
| | Propylparaben | | | | | | | | | |
| | (and) Isobutyl- | | | | | | | | | |
| | paraben | | | | | | | | | |

Example 8: Sun Cream

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Tribehenin PEG-20 esters | 5.00 | | | 5.00 | | | 5.00 | | |
| | Dibutyl adipate | 5.00 | 5.00 | 5.00 | 5.00 | 5.00 | 5.00 | 5.00 | 5.00 | 5.00 |
| | Octadecene/MA Copolymer | | | | 3.00 | | | | | |
| | (and) Methyl Acethyl | | | | | | | | | |
| | Ricinoleate | | | | | | | | | |
| | (and) Di-methylhepthyl | | | | | | | | | |
| | Adipate | | | | | | | | | |
| | C30-38 olefin/isopropyl | | | | | 2.00 | | | | |
| | maleate/MA copolymer | | | | | | | | | |
| | Hydrogenated Dimer Dilinoleyl/ | | | | | | 3.00 | | | |
| | Dimethylcarbonate Co- | | | | | | | | | |
| | polymer | | | | | | | | | |
| | Cyclopentasiloxane (and) | | | | | | | 7.00 | | |
| | Acrylates/ | | | | | | | | | |
| | Polytrimethylsiloxymethacrylate | | | | | | | | | |
| | Copolymer | | | | | | | | | |
| | Isododecane (and) Acrylates/ | | | | | | | | 8.00 | |
| | Polytrimethylsiloxymethacrylate | | | | | | | | | |
| | Copolymer | | | | | | | | | |
| | Poly(Glycol Adipate)/Bis- | | | | | | | | | 5.00 |
| | Hydroxyethyoxypropyl | | | | | | | | | |
| | Dimethicone | | | | | | | | | |
| | Copolymer | | | | | | | | | |
| | PPG-2 Myristyl Ether | 5.00 | 5.00 | 5.00 | 5.00 | 5.00 | 5.00 | 5.00 | 5.00 | 5.00 |
| | Propionate | | | | | | | | | |
| | Octocrylene | 6.00 | 6.00 | 6.00 | 6.00 | 6.00 | 6.00 | 6.00 | 6.00 | 6.00 |
| | Butyl Methoxydibenzoyl- | 2.50 | 2.50 | 2.50 | 2.50 | 2.50 | 2.50 | 2.50 | 2.50 | 2.50 |
| | methane | | | | | | | | | |
| | Pentaerythrityl Distearate | | 1.50 | | | | | 3.00 | | 5.00 |
| | Glyceryl Dibehenate (and) | | | 2.00 | | 4.00 | | | | |
| | Tribehenin (and) Glyceryl | | | | | | | | | |
| | Behenate | | | | | | | | | |
| | Ethylhexyl Methoxycinnamate | 2.00 | 2.00 | 2.00 | 2.00 | 2.00 | 2.00 | 2.00 | 2.00 | 2.00 |
| | Sorbitan stearate (and) Sorbitol | | 5.00 | | | 5.00 | | | 5.00 | |
| | monolaurate | | | | | | | | | |
| | Sucrose polystearate | | | 5.00 | | | 5.00 | | | 5.00 |
| | Dimethyl capramide | 2.00 | 4.00 | 6.00 | 8.00 | 9.00 | 12.00 | 14.00 | 16.00 | 10.00 |
| | Bis-Ethylhexyloxyphenol | 1.00 | 2.00 | 3.00 | 4.00 | 5.00 | 6.00 | 7.00 | 2.00 | 4.00 |
| | Methoxyphenyl | | | | | | | | | |
| | Triazine (Tinosorb | | | | | | | | | |
| | S) | | | | | | | | | |
| Part B | Water | Qs to 100 | Qs to 100 | Qs to 100 | Qs to 100 | Qs to 100 | Qs to 100 | Qs to 100 | Qs to 100 | Qs to 100 |
| | PVP/dimethylconylacrylate/ | 3.00 | | | | | | | | |
| | polycarbamyl/ | | | | | | | | | |
| | polyglycol ester | | | | | | | | | |
| | Polyester-5 | | 5.00 | | | | | | | |
| | Disteareth-75 IPDI | | 0.25 | 3.00 | | | | | | |
| | Disteareth-100 IPDI | | | | 0.25 | 3.00 | | | | |
| | Acrylates/Vinyl Neodecanoate | | | | | | 1.50 | 5.00 | 8.00 | |
| | Crosspolymer | | | | | | | | | |
| | Disodium EDTA | 0.20 | 0.20 | 0.20 | 0.20 | 0.20 | 0.20 | 0.20 | 0.20 | 0.20 |
| | Sclerotium Gum | 2.00 | 2.00 | 2.00 | 2.00 | 2.00 | 2.00 | 2.00 | 2.00 | 2.00 |
| | Ammonium | 0.50 | 0.50 | 0.50 | 0.50 | 0.50 | | | | 0.50 |
| | Acryldimethyltaurate/ | | | | | | | | | |
| | Beneth-25 Methacrylate | | | | | | | | | |
| | Crosspolymer. | | | | | | | | | |
| Part C | Cyclopentasiloxane (and) | 4.00 | 4.00 | 4.00 | 4.00 | 4.00 | 4.00 | 4.00 | 4.00 | 4.00 |
| | cyclohexasiloxane | | | | | | | | | |
| Part D | Water | 5.00 | 5.00 | 5.00 | 5.00 | 5.00 | 5.00 | 5.00 | 5.00 | 5.00 |
| | Tromethamine | 0.90 | 0.90 | 0.90 | 0.90 | 0.90 | 0.90 | 0.90 | 0.90 | 0.90 |
| | Phenylbenzimidazole Sulfonic | 2.00 | 2.00 | 2.00 | 2.00 | 2.00 | 2.00 | 2.00 | 2.00 | 2.00 |
| | Acid | | | | | | | | | |
| Part E | Methylene Bis-Benzotriazolyl | 10.00 | 10.00 | 10.00 | 10.00 | 10.00 | 10.00 | 10.00 | 10.00 | 10.00 |
| | Tetramethylbutylphenol (and) | | | | | | | | | |
| | Aqua (and) Decyl Glucoside | | | | | | | | | |
| | (and) Propylene Glycol (and) | | | | | | | | | |
| | Xanthan Gum | | | | | | | | | |
| Part F | Aqua (and) Tocopheryl Acetate | 0.50 | 0.50 | 0.50 | 0.50 | 0.50 | 0.50 | 0.50 | 0.50 | 0.50 |
| | (and) Caprylic/Capric | | | | | | | | | |
| | Triglyceride | | | | | | | | | |
| | (and) Polysorbate 80 | | | | | | | | | |
| | (and) Lecithin | | | | | | | | | |
| | Phenoxyethanol (and) Methyl- | 1.00 | 1.00 | 1.00 | 1.00 | 1.00 | 1.00 | 1.00 | 1.00 | 1.00 |
| | paraben (and) Ethylparaben | | | | | | | | | |
| | (and) Butylparaben (and) | | | | | | | | | |
| | Propylparaben | | | | | | | | | |
| | (and) Isobutylparaben | | | | | | | | | |
| Part G | Sodium Hydroxide (and) Water | q.s.p. | q.s.p. | q.s.p. | q.s.p. | q.s.p. | q.s.p. | q.s.p. | q.s.p. | q.s.p. |
| | | pH = 7 | pH = 7 | pH = 7 | pH = 7 | pH = 7 | pH = 7 | pH = 7 | pH = 7 | pH = 7 |

Example 9: Sun Cream

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Behenyl Alcohol (and) Glyceryl | 5.25 | | | 5.25 | | | 5.25 | | |
| | Stearate (and) Glyceryl Stearate | | | | | | | | | |
| | Citrate (and) Disodium | | | | | | | | | |
| | Ethylene Di(Cocamide PEG-15 | | | | | | | | | |
| | Disulfate) | | | | | | | | | |
| | Sorbitan stearate (and) Sorbitol | | 5.00 | | | 5.00 | | | 5.00 | |
| | monolaurate | | | | | | | | | |
| | Sucrose polystearate | | | 5.00 | | | 5.00 | | | 5.00 |
| | Ethylhexyl Methoxycinnamate | 6.00 | 6.00 | 6.00 | 6.00 | 6.00 | 6.00 | 6.00 | 6.00 | 6.00 |
| | Butyl Methoxydibenzoyl- | 1.00 | 1.00 | 1.00 | 1.00 | 1.00 | 1.00 | 1.00 | 1.00 | 1.00 |
| | methane | | | | | | | | | |
| | Tridecyl Salicylate | 5.00 | 5.00 | 5.00 | 5.00 | 5.00 | 5.00 | 5.00 | 5.00 | 5.00 |
| | Octadecene/MA Copolymer | | | | 3.00 | | | | | |
| | (and) Methyl Acethyl | | | | | | | | | |
| | Ricinoleate | | | | | | | | | |
| | (and) Di-methylhepthyl | | | | | | | | | |
| | Adipate | | | | | | | | | |
| | C30-38 olefin/isopropyl | | | | | 2.00 | | | | |
| | maleate/MA copolymer | | | | | | | | | |
| | Hydrogenated Dimer Dilinoleyl/ | | | | | | 3.00 | | | |
| | Dimethylcarbonate Co- | | | | | | | | | |
| | polymer | | | | | | | | | |
| | Cyclopentasiloxane (and) | | | | | | | 7.00 | | |
| | Acrylates/ | | | | | | | | | |
| | Polytrimethylsiloxymethacrylate | | | | | | | | | |
| | Copolymer | | | | | | | | | |
| | Isododecane (and) Acrylates/ | | | | | | | | 8.00 | |
| | Polytrimethylsiloxymethacrylate | | | | | | | | | |
| | Copolymer | | | | | | | | | |
| | Poly(Glycol Adipate)/Bis- | | | | | | | | | 5.00 |
| | Hydroxyethyoxypropyl | | | | | | | | | |
| | Dimethicone Copolymer | | | | | | | | | |
| | Ethylhexyl Palmitate | 2.00 | 2.00 | 2.00 | 2.00 | 2.00 | 2.00 | 2.00 | 2.00 | 2.00 |
| | Pentaerythrityl Distearate | | 1.50 | | | | | 3.00 | | 5.00 |
| | Glyceryl Dibehenate (and) Tri- | | | 2.00 | | 4.00 | | | | |
| | behenin (and) Glyceryl Behenate | | | | | | | | | |
| | Cyclopentasiloxane (and) | 1.00 | 1.00 | 1.00 | 1.00 | 1.00 | 1.00 | 1.00 | 1.00 | 1.00 |
| | Cyclohexasiloxane | | | | | | | | | |
| | Dimethyl capramide | 2.00 | 4.00 | 6.00 | 8.00 | 9.00 | 12.00 | 14.00 | 16.00 | 10.00 |
| | Bis-Ethylhexyloxyphenol | 1.00 | 2.00 | 3.00 | 4.00 | 5.00 | 6.00 | 7.00 | 2.00 | 4.00 |
| | Methoxyphenyl | | | | | | | | | |
| | Triazine (Tinosorb S) | | | | | | | | | |
| Part B | Aqua | Qs to 100 | Qs to 100 | Qs to 100 | Qs to 100 | Qs to 100 | Qs to 100 | Qs to 100 | Qs to 100 | Qs to 100 |
| | PVP/dimethylconylacrylate/ | 3.00 | | | | | | | | |
| | polycarbamyl/ | | | | | | | | | |
| | polyglycol ester | | | | | | | | | |
| | Polyester-5 | | 5.00 | | | | | | | |
| | Disteareth-75 IPDI | | 0.25 | 3.00 | | | | | | |
| | Disteareth-100 IPDI | | | | 0.25 | 3.00 | | | | |
| | Acrylates/Vinyl Neodecanoate | | | | | | 1.50 | 5.00 | 8.00 | |
| | Crosspolymer | | | | | | | | | |
| | Xanthan Gum | 0.30 | 0.30 | 0.30 | 0.30 | 0.30 | 0.30 | 0.30 | 0.30 | 0.30 |
| | Trisodium EDTA | 0.20 | 0.20 | 0.20 | 0.20 | 0.20 | 0.20 | 0.20 | 0.20 | 0.20 |
| | Disodium Ethylene Di | 1.00 | 1.00 | 1.00 | 1.00 | 1.00 | 1.00 | 1.00 | 1.00 | 1.00 |
| | (Cocamide | | | | | | | | | |
| | PEG-15 Disulfate) (and) | | | | | | | | | |
| | Sodium Lauroyl Lactylate | | | | | | | | | |
| Part C | Carbomer | 0.25 | 0.25 | 0.25 | 0.25 | 0.25 | 0.25 | 0.25 | 0.25 | 0.25 |
| | water | 10.00 | 10.00 | 10.00 | 10.00 | 10.00 | 10.00 | 10.00 | 10.00 | 10.00 |
| | Tocopheryl Acetate | 1.00 | 1.00 | 1.00 | 1.00 | 1.00 | 1.00 | 1.00 | 1.00 | 1.00 |
| Part D | Phenoxyethanol (and) Methyl- | 0.70 | 0.70 | 0.70 | 0.70 | 0.70 | 0.70 | 0.70 | 0.70 | 0.70 |
| | paraben (and) Ethylparaben | | | | | | | | | |
| | (and) Butylparaben (and) | | | | | | | | | |
| | Propylparaben | | | | | | | | | |
| | (and) Isobutyl- | | | | | | | | | |
| | paraben | | | | | | | | | |
| | Sodium Hydoxide 10% | Qsp | Qsp | Qsp | Qsp | Qsp | Qsp | Qsp | Qsp | Qsp |

Example 10: O/W Sun Cream Nonionic

| | | A | B | C | D | E | F | G | H | I |
|---|---|---|---|---|---|---|---|---|---|---|
| | INCI-Name | % | % | % | % | % | % | % | % | % |
| Part A | Bis-PEG/PPG-16/16 PEG/- | 2.00 | | | 2.00 | | | 2.00 | | |
| | PPG-16/16 Dimethicone (and) | | | | | | | | | |
| | Caprylic/Capric Triglyceride | | | | | | | | | |
| | Sorbitan stearate (and) Sorbitol | | 5.00 | | | 5.00 | | | 5.00 | |
| | monolaurate | | | | | | | | | |
| | Sucrose polystearate | | | 5.00 | | | 5.00 | | | 5.00 |
| | Ethylhexyl Methoxycinnamate | 5.00 | 5.00 | 5.00 | 5.00 | 5.00 | 5.00 | 5.00 | 5.00 | 5.00 |
| | Isoamyl p-Methoxycinnamate | 5.00 | 5.00 | 5.00 | 5.00 | 5.00 | 5.00 | 5.00 | 5.00 | 5.00 |
| | Tocopheryl Acetate | 0.50 | 0.50 | 0.50 | 0.50 | 0.50 | 0.50 | 0.50 | 0.50 | 0.50 |
| | Octadecene/MA Copolymer | | | | 3.00 | | | | | |
| | (and) Methyl Acethyl | | | | | | | | | |
| | Ricinoleate | | | | | | | | | |
| | (and) Di-methylhepthyl | | | | | | | | | |
| | Adipate | | | | | | | | | |
| | C30-38 olefin/isopropyl | | | | | 2.00 | | | | |
| | maleate/MA copolymer | | | | | | | | | |
| | Hydrogenated Dimer Dilinoleyl/ | | | | | | 3.00 | | | |
| | Dimethylcarbonate Co- | | | | | | | | | |
| | polymer | | | | | | | | | |
| | Cyclopentasiloxane (and) | | | | | | | 7.00 | | |
| | Acryates/ | | | | | | | | | |
| | Polytrimethylsiloxymethacrylate | | | | | | | | | |
| | Copolymer | | | | | | | | | |
| | Isododecane (and) Acrylates/- | | | | | | | | 8.00 | |
| | Polytrimethylsiloxymethacrylate | | | | | | | | | |
| | Copolymer | | | | | | | | | |
| | Poly(Glycol Adipate)/Bis- | | | | | | | | | 5.00 |
| | Hydroxyethyoxypropyl | | | | | | | | | |
| | Dimethicone | | | | | | | | | |
| | Copolymer | | | | | | | | | |
| | Pentaerythrityl Distearate | | 1.50 | | | | | 3.00 | | 5.00 |
| | Glyceryl Dibehenate (and) | | | 2.00 | | 4.00 | | | | |
| | Tribehenin (and) Glyceryl | | | | | | | | | |
| | Behenate | | | | | | | | | |
| | Dimethyl capramide | 2.00 | 4.00 | 6.00 | 8.00 | 9.00 | 12.00 | 14.00 | 16.00 | 10.00 |
| | Bis-Ethylhexyloxyphenol | 1.00 | 2.00 | 3.00 | 4.00 | 5.00 | 6.00 | 7.00 | 2.00 | 4.00 |
| | Methoxyphenyl Triazine | | | | | | | | | |
| | (Tinosorb S) | | | | | | | | | |
| Part B | Aqua | Qs to 100 | Qs to 100 | Qs to 100 | Qs to 100 | Qs to 100 | Qs to 100 | Qs to 100 | Qs to 100 | Qs to 100 |
| | PVP/dimethylconylacrylate/ | 3.00 | | | | | | | | |
| | polycarbamyl/ | | | | | | | | | |
| | polyglycol ester | | | | | | | | | |
| | Polyester-5 | | 5.00 | | | | | | | |
| | Polysorbate 80 | 0.20 | 0.20 | 0.20 | 0.20 | 0.20 | 0.20 | 0.20 | 0.20 | 0.20 |
| | Disteareth-75 IPDI | | 0.25 | 3.00 | | | | | | |
| | Disteareth-100 IPDI | | | | 0.25 | 3.00 | | | | |
| | Acrylates/Vinyl Neodecanoate | | | | | | 1.50 | 5.00 | 8.00 | |
| | Crosspolymer | | | | | | | | | |
| | Trisodium EDTA | 0.15 | 0.15 | 0.15 | 0.15 | 0.15 | 0.15 | 0.15 | 0.15 | 0.15 |
| | Sodium Carboxymethyl | 0.10 | 0.10 | 0.10 | 0.10 | 0.10 | 0.10 | 0.10 | 0.10 | 0.10 |
| | Betaglucan | | | | | | | | | |
| Part C | Carbomer | 0.30 | 0.30 | 0.30 | 0.30 | 0.30 | 0.30 | 0.30 | 0.30 | 0.30 |
| | Xanthan Gum | 0.10 | 0.10 | 0.10 | 0.10 | 0.10 | 0.10 | 0.10 | 0.10 | 0.10 |
| | Isopropyl Palmitate | 1.60 | 1.60 | 1.60 | 1.60 | 1.60 | 1.60 | 1.60 | 1.60 | 1.60 |
| Part D | Methylene Bis-Benzotriazolyl | 4.00 | 4.00 | 4.00 | 4.00 | 4.00 | 4.00 | 4.00 | 4.00 | 4.00 |
| | Tetramethylbutylphenol (and) | | | | | | | | | |
| | Aqua (and) Decyl Glucoside | | | | | | | | | |
| | (and) Propylene Glycol (and) | | | | | | | | | |
| | Xanthan Gum | | | | | | | | | |
| Part E | Phenoxyethanol (and) Methyl- | 0.70 | 0.70 | 0.70 | 0.70 | 0.70 | 0.70 | 0.70 | 0.70 | 0.70 |
| | paraben (and) Ethylparaben | | | | | | | | | |
| | (and) Butylparaben (and) | | | | | | | | | |
| | Propylparaben | | | | | | | | | |
| | (and) Isobutylparaben | | | | | | | | | |
| | Sodium Hydoxide 10% | Qs | Qs | Qs | Qs | Qs | Qs | Qs | Qs | Qs |

Example 11: Sun Cream

| | | A | B | C | D | E | F | G | H | I |
|---|---|---|---|---|---|---|---|---|---|---|
| | INCI-Name | % | % | % | % | % | % | % | % | % |
| Part A | Cetearyl glucoside | 4.00 | | | 4.00 | | | 4.00 | | |
| | Dicaprylyl Carbonate | 1.00 | 1.00 | 1.00 | 1.00 | 1.00 | 1.00 | 1.00 | 1.00 | 1.00 |
| | Octocrylene | 10.00 | 10.00 | 10.00 | 10.00 | 10.00 | 10.00 | 10.00 | 10.00 | 10.00 |
| | Butyl Methoxydibenzoyl- | 4.00 | 4.00 | 4.00 | 4.00 | 4.00 | 4.00 | 4.00 | 4.00 | 4.00 |
| | methane | | | | | | | | | |
| | Ethylhexyl salicyate | 3.00 | 3.00 | 3.00 | 3.00 | 3.00 | 3.00 | 3.00 | 3.00 | 3.00 |
| | Neopentyl Glycol Diheptanoate | 6.00 | | 5.00 | | | | | | |
| | Propylene Glycol Dibenzoate | 4.00 | | | | | | | | |
| | Polyester-7 and Neopenthyl | | | | | 8.00 | | | | |
| | Glycol Diheptanoate | | | | | | | | | |
| | Diethylhexyl Syringylidene- | | 6.00 | | | | | | | |
| | malonate | | | | | | | | | |
| | Polyester 8 | | | 4.00 | | | | | | |
| | Diethylhexyl Malate | | | | 7.00 | | | | | |
| | PPG-3 Myristyl Ether | | | | | | 6.00 | | | |
| | Neoheptanoate | | | | | | | | | |
| | Phenethyl Benzoate | | | | | | | 8.00 | | |
| | Isopropyl PPG-2 Isodeceth-7 | | | | | | | | 4.00 | |
| | Carboxylate | | | | | | | | | |
| | Isopropyl C12-15-Pareth-9 | | | | | | | | 4.00 | |
| | Carboxylate | | | | | | | | | |
| | Cetyl PPG-2 Isodeceth-7 | | | | | | | | 4.00 | |
| | Carboxylate | | | | | | | | | |
| | Ethylhexyl Methoxycinnamate | 1.80 | | | 1.80 | | | 1.80 | | |
| | Sorbitan stearate (and) Sorbitol | | 5.00 | | | 5.00 | | | 5.00 | |
| | monolaurate | | | | | | | | | |
| | Sucrose polystearate | | | 5.00 | | | 5.00 | | | 5.00 |
| | Dimethyl capramide | 2.00 | 4.00 | 6.00 | 8.00 | 9.00 | 12.00 | 14.00 | 16.00 | 10.00 |
| | Bis-Ethylhexyloxyphenol | 1.00 | 2.00 | 3.00 | 4.00 | 5.00 | 6.00 | 7.00 | 2.00 | 4.00 |
| | Methoxyphenyl Triazine | | | | | | | | | |
| | (Tinosorb S) | | | | | | | | | |
| Part B | Water | Qs to 100 | Qs to 100 | Qs to 100 | Qs to 100 | Qs to 100 | Qs to 100 | Qs to 100 | Qs to 100 | Qs to 100 |
| | Disodium EDTA | 0.20 | 0.20 | 0.20 | 0.20 | 0.20 | 0.20 | 0.20 | 0.20 | 0.20 |
| | Sodium polyacrylate | 0.70 | 0.70 | 0.70 | 0.70 | 0.70 | 0.70 | 0.70 | 0.70 | 0.70 |
| Part C | Dimethicone | 3.00 | 3.00 | 3.00 | 3.00 | 3.00 | 3.00 | 3.00 | 3.00 | 3.00 |
| | Corn Starch modified | 3.00 | 3.00 | 3.00 | 3.00 | 3.00 | 3.00 | 3.00 | 3.00 | 3.00 |
| Part D | Methylene Bis-Benzotriazolyl | 4.00 | 4.00 | 4.00 | 4.00 | 4.00 | 4.00 | 4.00 | 4.00 | 4.00 |
| | Tetramethylbutylphenol (and) | | | | | | | | | |
| | Aqua (and) Decyl Glucoside | | | | | | | | | |
| | (and) Propylene Glycol (and) | | | | | | | | | |
| | Xanthan Gum | | | | | | | | | |
| Part E | Phenoxyethanol (and) Methyl- | 1.00 | 1.00 | 1.00 | 1.00 | 1.00 | 1.00 | 1.00 | 1.00 | 1.00 |
| | paraben (and) Ethylparaben | | | | | | | | | |
| | (and) Butylparaben (and) | | | | | | | | | |
| | Propylparaben | | | | | | | | | |
| | (and) Isobutylparaben | | | | | | | | | |

Example 12: O/W Sun Cream Nonionic

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Bis-PEG/PPG-16/16 | 2.00 | | | 2.00 | | | 2.00 | | |
| | PEG/PPG-16/16 Dimethicone | | | | | | | | | |
| | (and) Caprylic/Capric | | | | | | | | | |
| | Triglyceride | | | | | | | | | |
| | Sorbitan stearate (and) Sorbitol | | 5.00 | | | 5.00 | | | 5.00 | |
| | monolaurate | | | | | | | | | |
| | Sucrose polystearate | | | 5.00 | | | 5.00 | | | 5.00 |
| | Neopentyl Glycol Diheptanoate | 6.00 | | 5.00 | | | | | | |
| | Propylene Glycol Dibenzoate | 4.00 | | | | | | | | |
| | Polyester-7 and Neopenthyl | | | | | 8.00 | | | | |
| | Glycol Diheptanoate | | | | | | | | | |
| | Diethylhexyl Syringylidene- | | 6.00 | | | | | | | |
| | malonate | | | | | | | | | |
| | Polyester 8 | | | 4.00 | | | | | | |
| | Diethylhexyl Malate | | | | 7.00 | | | | | |
| | PPG-3 Myristyl Ether | | | | | | 6.00 | | | |
| | Neoheptanoate | | | | | | | | | |
| | Phenethyl Benzoate | | | | | | | 8.00 | | |
| | Isopropyl PPG-2 Isodeceth-7 | | | | | | | | 4.00 | |
| | Carboxylate | | | | | | | | | |
| | Isopropyl C12-15-Pareth-9 | | | | | | | | 4.00 | |
| | Carboxylate | | | | | | | | | |
| | Cetyl PPG-2 Isodeceth-7 | | | | | | | | 4.00 | |
| | Carboxylate | | | | | | | | | |
| | Ethylhexyl Methoxycinnamate | 5.00 | 5.00 | 5.00 | 5.00 | 5.00 | 5.00 | 5.00 | 5.00 | 5.00 |
| | Isoamyl p-Methoxycinnamate | 5.00 | 5.00 | 5.00 | 5.00 | 5.00 | 5.00 | 5.00 | 5.00 | 5.00 |
| | Tocopheryl Acetate | 0.50 | 0.50 | 0.50 | 0.50 | 0.50 | 0.50 | 0.50 | 0.50 | 0.50 |
| | Avocado (Persea Gratissima) | 1.00 | 1.00 | 1.00 | 1.00 | 1.00 | 1.00 | 1.00 | 1.00 | 1.00 |
| | Oil | | | | | | | | | |
| | Isopropyl Palmitate | 0.40 | 0.40 | 0.40 | 0.40 | 0.40 | 0.40 | 0.40 | 0.40 | 0.40 |
| | Decyl Cocoate | 1.00 | 1.00 | 1.00 | 1.00 | 1.00 | 1.00 | 1.00 | 1.00 | 2.00 |
| | C12-15 Alkyl Benzoate | 1.00 | 1.00 | 1.00 | 1.00 | 1.00 | 1.00 | 1.00 | 1.00 | 3.00 |
| | Dimethyl capramide | 2.00 | 4.00 | 6.00 | 8.00 | 9.00 | 12.00 | 14.00 | 16.00 | 10.00 |
| | Bis-Ethylhexyloxyphenol | 1.00 | 2.00 | 3.00 | 4.00 | 5.00 | 6.00 | 7.00 | 2.00 | 4.00 |
| | Methoxyphenyl | | | | | | | | | |
| | Triazine (Tinosorb S) | | | | | | | | | |
| Part B | Aqua | Qs to 100 | Qs to 100 | Qs to 100 | Qs to 100 | Qs to 100 | Qs to 100 | Qs to 100 | Qs to 100 | Qs to 100 |
| | Glycerin | 2.00 | 2.00 | 2.00 | 2.00 | 2.00 | 2.00 | 2.00 | 2.00 | 2.00 |
| | Polysorbate 80 | 0.20 | 0.20 | 0.20 | 0.20 | 0.20 | 0.20 | 0.20 | 0.20 | 0.20 |
| | Trisodium EDTA | 0.15 | 0.15 | 0.15 | 0.15 | 0.15 | 0.15 | 0.15 | 0.15 | 0.15 |
| | Sodium Carboxymethyl | 0.10 | 0.10 | 0.10 | 0.10 | 0.10 | 0.10 | 0.10 | 0.10 | 0.10 |
| | Betaglucan | | | | | | | | | |
| Part C | Carbomer | 0.30 | 0.30 | 0.30 | 0.30 | 0.30 | 0.30 | 0.30 | 0.30 | 0.30 |
| | Xanthan Gum | 0.10 | 0.10 | 0.10 | 0.10 | 0.10 | 0.10 | 0.10 | 0.10 | 0.10 |
| | Isopropyl Palmitate | 1.60 | 1.60 | 1.60 | 1.60 | 1.60 | 1.60 | 1.60 | 1.60 | 1.60 |
| Part D | Methylene Bis-Benzotriazolyl | 8.00 | 8.00 | 8.00 | 8.00 | 8.00 | 8.00 | 8.00 | 8.00 | 8.00 |
| | Tetramethylbutylphenol (and) | | | | | | | | | |
| | Aqua (and) Decyl Glucoside | | | | | | | | | |
| | (and) Propylene Glycol (and) | | | | | | | | | |
| | Xanthan Gum | | | | | | | | | |
| Part E | Phenoxyethanol (and) Methyl- | 0.70 | 0.70 | 0.70 | 0.70 | 0.70 | 0.70 | 0.70 | 0.70 | 0.70 |
| | paraben (and) Ethylparaben | | | | | | | | | |
| | (and) Butylparaben (and) | | | | | | | | | |
| | Propylparaben | | | | | | | | | |
| | (and) Isobutylparaben | | | | | | | | | |
| | Sodium Hydoxide 10% | Qs | Qs | Qs | Qs | Qs | Qs | Qs | Qs | Qs |

Example 13: Sun Cream

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Cetearyl glucoside | 4.00 | | | 4.00 | | | 4.00 | | |
| | Dicaprylyl Carbonate | 8.00 | 8.00 | 8.00 | 8.00 | 8.00 | 8.00 | 8.00 | 8.00 | 8.00 |
| | Octocrylene | 10.00 | 10.00 | 10.00 | 10.00 | 10.00 | 10.00 | 10.00 | 10.00 | 10.00 |
| | Butyl Methoxydibenzoyl- | 4.00 | 4.00 | 4.00 | 4.00 | 4.00 | 4.00 | 4.00 | 4.00 | 4.00 |
| | methane | | | | | | | | | |
| | Butylene Glycol Cocoate | 4.00 | 6.00 | 4.00 | 3.00 | 2.00 | 1.00 | 2.50 | 2.00 | 4.00 |
| | Ethylhexyl salicyate | 3.00 | 3.00 | 3.00 | 3.00 | 3.00 | 3.00 | 3.00 | 3.00 | 3.00 |
| | Glyceryl Dibehenate (and) | | | 2.00 | | 4.00 | | | | |
| | Tribehenin (and) Glyceryl | | | | | | | | | |
| | Behenate | | | | | | | | | |
| | Ethylhexyl Methoxycinnamate | 1.80 | 1.80 | 1.80 | 1.80 | 1.80 | 1.80 | 1.80 | 1.80 | 1.80 |
| | Sorbitan stearate (and) Sorbitol | | 5.00 | | | 5.00 | | | 5.00 | |
| | monolaurate | | | | | | | | | |
| | Sucrose polystearate | | | 5.00 | | | 5.00 | | | 5.00 |
| | Dimethyl capramide | 2.00 | 4.00 | 6.00 | 8.00 | 9.00 | 12.00 | 14.00 | 16.00 | 10.00 |
| | Bis-Ethylhexyloxyphenol | 1.00 | 2.00 | 3.00 | 4.00 | 5.00 | 6.00 | 7.00 | 2.00 | 4.00 |
| | Methoxyphenyl Triazine | | | | | | | | | |
| | (Tinosorb S) | | | | | | | | | |
| Part B | Water | Qs to 100 | Qs to 100 | Qs to 100 | Qs to 100 | Qs to 100 | Qs to 100 | Qs to 100 | Qs to 100 | Qs to 100 |
| | Disodium EDTA | 0.20 | 0.20 | 0.20 | 0.20 | 0.20 | 0.20 | 0.20 | 0.20 | 0.20 |
| | Glycerin | 2.00 | 2.00 | 2.00 | 2.00 | 2.00 | 2.00 | 2.00 | 2.00 | 2.00 |
| | Sodium polyacrylate | 0.70 | 0.70 | 0.70 | 0.70 | 0.70 | | | | 0.70 |
| Part C | Dimethicone | 3.00 | 3.00 | 3.00 | 3.00 | 3.00 | 3.00 | 3.00 | 3.00 | 3.00 |
| | Corn Starch modified | 3.00 | 3.00 | 3.00 | 3.00 | 3.00 | 3.00 | 3.00 | 3.00 | 3.00 |
| Part D | Methylene Bis-Benzotriazolyl | 4.00 | 4.00 | 4.00 | 4.00 | 4.00 | 4.00 | 4.00 | 4.00 | 4.00 |
| | Tetramethylbutylphenol (and) | | | | | | | | | |
| | Aqua (and) Decyl Glucoside | | | | | | | | | |
| | (and) Propylene Glycol (and) | | | | | | | | | |
| | Xanthan Gum | | | | | | | | | |
| | Styrene/Acrylates Copolymer | 5.00 | | | | | 3.00 | | 1.00 | |
| | PEG-6 Isostearate (and) | | 3.00 | | | | 1.00 | 4.00 | | 1.00 |
| | Hesperetin | | | | | | | | | |
| | Laurate | | | | | | | | | |
| | Polyacrylate-15 (and) Poly- | | | 4.00 | | | | | 2.00 | |
| | acrylate-17 | | | | | | | | | |
| | Carthamus Tinctorius | | | | 5.00 | | | | | 2.00 |
| | (Safflower) | | | | | | | | | |
| | Oleosomes | | | | | | | | | |
| | PTFE | | | | | 4.00 | | | | 2.00 |
| Part E | Phenoxyethanol (and) Methyl- | 1.00 | 1.00 | 1.00 | 1.00 | 1.00 | 1.00 | 1.00 | 1.00 | 1.00 |
| | paraben (and) Ethylparaben | | | | | | | | | |
| | (and) Butylparaben (and) | | | | | | | | | |
| | Propylparaben | | | | | | | | | |
| | (and) Isobutylparaben | | | | | | | | | |

Example 14: O/W Sun Cream Nonionic

| | | A | B | C | D | E | F | G | H | I |
|---|---|---|---|---|---|---|---|---|---|---|
| | INCI-Name | % | % | % | % | % | % | % | % | % |
| Part A | Bis-PEG/PPG-16/16 | 2.00 | | | 2.00 | | | 2.00 | | |
| | PEG/PPG-16/16 Dimethicone | | | | | | | | | |
| | (and) Caprylic/Capric Triglyceride | | | | | | | | | |
| | Sorbitan stearate (and) Sorbitol | | 5.00 | | | 5.00 | | | 5.00 | |
| | monolaurate | | | | | | | | | |
| | Sucrose polystearate | | | 5.00 | | | 5.00 | | | 5.00 |
| | Ethylhexyl Methoxycinnamate | 5.00 | 5.00 | 5.00 | 5.00 | 5.00 | 5.00 | 5.00 | 5.00 | 5.00 |
| | Butylene Glycol Cocoate | 4.00 | 6.00 | 4.00 | 3.00 | 2.00 | 1.00 | 2.50 | 2.00 | 4.00 |
| | Isoamyl p-Methoxycinnamate | 5.00 | 5.00 | 5.00 | 5.00 | 5.00 | 5.00 | 5.00 | 5.00 | 5.00 |
| | Tocopheryl Acetate | 0.50 | 0.50 | 0.50 | 0.50 | 0.50 | 0.50 | 0.50 | 0.50 | 0.50 |
| | Avocado (Persea Gratissima) | 1.00 | 1.00 | 1.00 | 1.00 | 1.00 | 1.00 | 1.00 | 1.00 | 1.00 |
| | Oil | | | | | | | | | |
| | Pentaerythrityl Distearate | | 1.50 | | | | | 3.00 | | 5.00 |
| | Glyceryl Dibehenate (and) | | | 2.00 | | 4.00 | | | | |
| | Tribehenin (and) Glyceryl | | | | | | | | | |
| | Behenate | | | | | | | | | |
| | Decyl Cocoate | 2.00 | 2.00 | 2.00 | 2.00 | 2.00 | 2.00 | 2.00 | 2.00 | 2.00 |
| | Dimethyl capramide | 2.00 | 4.00 | 6.00 | 8.00 | 9.00 | 12.00 | 14.00 | 16.00 | 10.00 |
| | Bis-Ethylhexyloxyphenol Methoxyphenyl | 1.00 | 2.00 | 3.00 | 4.00 | 5.00 | 6.00 | 7.00 | 2.00 | 4.00 |
| | Triazine (Tinosorb S) | | | | | | | | | |
| Part B | Aqua | Qs to | Qs to | Qs to | Qs to | Qs to | Qs to | Qs to | Qs to | Qs to |
| | | 100 | 100 | 100 | 100 | 100 | 100 | 100 | 100 | 100 |
| | Polysorbate 80 | 0.20 | 0.20 | 0.20 | 0.20 | 0.20 | 0.20 | 0.20 | 0.20 | 0.20 |
| | Trisodium EDTA | 0.15 | 0.15 | 0.15 | 0.15 | 0.15 | 0.15 | 0.15 | 0.15 | 0.15 |
| | Sodium Carboxymethyl Betaglucan | 0.10 | 0.10 | 0.10 | 0.10 | 0.10 | 0.10 | 0.10 | 0.10 | 0.10 |
| Part C | Carbomer | 0.30 | 0.30 | 0.30 | 0.30 | 0.30 | 0.30 | 0.30 | 0.30 | 0.30 |
| | Xanthan Gum | 0.10 | 0.10 | 0.10 | 0.10 | 0.10 | 0.10 | 0.10 | 0.10 | 0.10 |
| | Isopropyl Palmitate | 1.60 | 1.60 | 1.60 | 1.60 | 1.60 | 1.60 | 1.60 | 1.60 | 1.60 |
| Part D | Methylene Bis-Benzotriazolyl | 5.00 | 5.00 | 5.00 | 5.00 | 5.00 | 5.00 | 5.00 | 5.00 | 5.00 |
| | Tetramethylbutylphenol (and) | | | | | | | | | |
| | Aqua (and) Decyl Glucoside | | | | | | | | | |
| | (and) Propylene Glycol (and) | | | | | | | | | |
| | Xanthan Gum | | | | | | | | | |
| Part E | Phenoxyethanol (and) Methylparaben | 0.70 | 0.70 | 0.70 | 0.70 | 0.70 | 0.70 | 0.70 | 0.70 | 0.70 |
| | (and) Ethylparaben | | | | | | | | | |
| | (and) Butylparaben (and) | | | | | | | | | |
| | Propylparaben (and) Isobutyl- | | | | | | | | | |
| | paraben | | | | | | | | | |
| | Styrene/Acrylates Copolymer | 5.00 | | | | | 3.00 | | 1.00 | |
| | PEG-6 Isostearate (and) Hesperetin | | 3.00 | | | | 1.00 | 4.00 | | 1.00 |
| | Laurate | | | | | | | | | |
| | Polyacrylate-15 (and) Poly- | | | 4.00 | | | | | 2.00 | |
| | acrylate-17 | | | | | | | | | |
| | Carthamus Tinctorius (Safflower) | | | | 5.00 | | | | | 2.00 |
| | Oleosomes | | | | | | | | | |
| | PTFE | | | | | 4.00 | | | | 2.00 |
| | Sodium Hydoxide 10% | Qs | Qs | Qs | Qs | Qs | Qs | Qs | Qs | Qs |

Example 15: Clear Sunscreen Gel

| | | A | B | C | D | E | F | G | H | I |
|---|---|---|---|---|---|---|---|---|---|---|
| Part | Ingredient | Wt. % | Wt. % | Wt. % | Wt. % | Wt. % | Wt. % | Wt. % | Wt. % | Wt. % |
| A | SD Alcohol 40 | Qs to | Qs to | Qs to | Qs to | Qs to | Qs to | Qs to | Qs to | Qs to |
| | | 100 | 100 | 100 | 100 | 100 | 100 | 100 | 100 | 100 |
| A | Dimethyl capramide | 2.00 | 4.00 | 6.00 | 8.00 | 9.00 | 12.00 | 14.00 | 16.00 | 10.00 |
| A | Bis-Ethylhexyloxyphenol Methoxyphenyl | 1.00 | 2.00 | 3.00 | 4.00 | 5.00 | 6.00 | 7.00 | 2.00 | 4.00 |
| | Triazine (Tinosorb S) | | | | | | | | | |
| A | Hydroxypropyl Cellulose | 1.70 | 1.70 | 1.70 | 1.70 | 1.70 | 1.70 | 1.70 | 1.70 | 1.70 |
| B | Isopropyl PPG-2 Isodeceth-7 | 9.70 | 9.70 | 9.70 | 9.70 | 9.70 | 9.70 | 9.70 | 9.70 | 9.70 |
| | Carboxylate | | | | | | | | | |
| B | Ethylhexyl Methoxycinnamate | 7.50 | 7.50 | 7.50 | 7.50 | 7.50 | 7.50 | 7.50 | 7.50 | 7.50 |
| B | Butyl Methoxydibenzoyl- | 1.00 | 1.00 | 1.00 | 1.00 | 1.00 | 1.00 | 1.00 | 1.00 | 1.00 |
| | methane | | | | | | | | | |
| B | Ethylhexyl Triazone | 1.50 | 1.50 | 1.50 | 1.50 | 1.50 | 1.50 | 1.50 | 1.50 | 1.50 |
| B | Benzophenone-3 | 2.00 | 2.00 | 2.00 | 2.00 | 2.00 | 2.00 | 2.00 | 2.00 | 2.00 |
| B | SD Alcohol 40 | 24.00 | 24.00 | 24.00 | 24.00 | 24.00 | 24.00 | 24.00 | 24.00 | 24.00 |
| B | Fragrance | 0.60 | 0.60 | 0.60 | 0.60 | 0.60 | 0.60 | 0.60 | 0.60 | 0.60 |
| C | Aqua | 8.00 | 8.00 | 8.00 | 8.00 | 8.00 | 8.00 | 8.00 | 8.00 | 8.00 |

Example 16: Sun Spray

| | | A | B | C | D | E | F | G | H | I |
|---|---|---|---|---|---|---|---|---|---|---|
| | INCI-Name | Wt. % | Wt. % | Wt. % | Wt. % | Wt. % | Wt. % | Wt. % | Wt. % | Wt. % |
| Part A | Butyl Methoxydibenzoyl- | 4.50 | 4.50 | 4.50 | 4.50 | 4.50 | 4.50 | 4.50 | 4.50 | 4.50 |
| | methane | | | | | | | | | |
| | Ethylhexyl Methoxycinnamate | 9.80 | 9.30 | 0.50 | 9.80 | 9.30 | 0.50 | 9.80 | 9.30 | 0.50 |
| | Octocrylene | | | 3.50 | | | 3.50 | | | 3.50 |
| | Ethylhexyl Salicylate | | | 1.80 | | | 1.80 | | | 1.80 |
| | Homosalate | | | 7.40 | | | 7.40 | | | 7.40 |
| | Diethylhexyl Butamido triazone | | | 2.50 | | | 2.50 | | | 2.50 |
| | Butylene Glycol Dicaprylate/ | 10.00 | 10.00 | 7.00 | 10.00 | 10.00 | 7.00 | 10.00 | 10.00 | 7.00 |
| | Dicaprate | | | | | | | | | |
| | C18-36 Acid Triglyceride | 0.70 | 0.70 | 0.70 | 0.70 | 0.70 | 0.70 | 0.70 | 0.70 | 0.70 |
| | Ceteareth-20 | 0.60 | 0.60 | 0.65 | 0.60 | 0.60 | 0.65 | 0.60 | 0.60 | 0.65 |
| | C12-15 Alkyl Benzoate | | | | | | | | | |
| | Taurine | 0.40 | 0.40 | | 0.40 | 0.40 | | 0.40 | 0.40 | |
| | Titanium Dioxide particles size | | | 1.40 | | | 1.40 | | | 1.40 |
| | 10 to 100 nm | | | | | | | | | |
| | PVP/Hexadecene Copolymer | 0.50 | 0.50 | 0.50 | 0.50 | 0.50 | 0.50 | 0.50 | 0.50 | 0.50 |
| | Dimethyl capramide | 2.00 | 4.00 | 6.00 | 8.00 | 9.00 | 12.00 | 14.00 | 16.00 | 10.00 |
| | Bis-Ethylhexyloxyphenol Methoxyphenyl | 1.00 | 2.00 | 3.00 | 4.00 | 5.00 | 6.00 | 7.00 | 2.00 | 4.00 |
| | Triazine (Tinosorb S) | | | | | | | | | |
| Part B | Aqua | Qs to | Qs to | Qs to | Qs to | Qs to | Qs to | Qs to | Qs to | Qs to |
| | | 100 | 100 | 100 | 100 | 100 | 100 | 100 | 100 | 100 |
| | Methylpropanediol | 1.00 | | | 1.00 | | | 1.00 | | |
| | Phenylbenzimidazole Sulfonic | | 1.00 | 1.50 | | 1.00 | 1.50 | | 1.00 | 1.50 |
| | Acid | | | | | | | | | |
| | Acrylates/C10-30 Alkyl Acrylate | 0.18 | 0.20 | 0.25 | 0.18 | 0.20 | 0.25 | 0.18 | 0.20 | 0.25 |
| | Crosspolymer | | | | | | | | | |
| | Trisodium EDTA | 0.10 | 0.10 | 0.10 | 0.10 | 0.10 | 0.10 | 0.10 | 0.10 | 0.10 |
| Part C | Alcohol Denatured | 2.00 | 2.00 | 2.00 | 2.00 | 2.00 | 2.00 | 2.00 | 2.00 | 2.00 |
| Part D | Phenoxyethanol (and) Methyl- | 0.70 | 0.70 | 0.70 | 0.70 | 0.70 | 0.70 | 0.70 | 0.70 | 0.70 |
| | paraben (and) Ethylparaben | | | | | | | | | |
| | (and) Butylparaben (and) Propylparaben | | | | | | | | | |
| | (and) Isobutylparaben | | | | | | | | | |
| | Tocopheryl Acetate | 0.50 | 0.50 | 0.50 | 0.50 | 0.50 | 0.50 | 0.50 | 0.50 | 0.50 |

Example 17: Sun Spray

| | | A | B | C | D | E | F | G | H | I |
|---|---|---|---|---|---|---|---|---|---|---|
| | INCI-Name | Wt. % | Wt. % | Wt. % | Wt. % | Wt. % | Wt. % | Wt. % | Wt. % | Wt. % |
| Part A | Behenyl Alcohol (and) Glyceryl | 0.50 | 0.50 | 0.50 | 0.50 | 0.50 | 0.50 | 0.50 | 0.50 | 0.50 |
| | Stearate (and) Glyceryl Stearate | | | | | | | | | |
| | Citrate (and) Disodium | | | | | | | | | |
| | Ethylene Di(Cocamide PEG- | | | | | | | | | |
| | 15 Disulfate) | | | | | | | | | |
| | Ethylhexyl Methoxycinnamate | 5.00 | 5.00 | 5.00 | 5.00 | 5.00 | 5.00 | 5.00 | 5.00 | 5.00 |
| | Butyl Methoxydibenzoyl- | 0.50 | 0.50 | 0.50 | 0.50 | 0.50 | 0.50 | 0.50 | 0.50 | 0.50 |
| | methane | | | | | | | | | |
| | Isotrideceth-12 | 0.50 | 0.50 | 0.50 | 0.50 | 0.50 | 0.50 | 0.50 | 0.50 | 0.50 |
| | Ethylhexyl Salicylate | 4.00 | 4.00 | 4.00 | 4.00 | 4.00 | 4.00 | 4.00 | 4.00 | 4.00 |
| | Hydrogenated Coco- | 1.50 | 1.50 | 1.50 | 1.50 | 1.50 | 1.50 | 1.50 | 1.50 | 1.50 |
| | glycerides | | | | | | | | | |
| | C12-15 Alkyl Benzoate | 8.00 | 8.00 | 8.00 | 8.00 | 8.00 | 8.00 | 8.00 | 8.00 | 8.00 |
| | Dimethyl capramide | 2.00 | 4.00 | 6.00 | 8.00 | 9.00 | 12.00 | 14.00 | 16.00 | 10.00 |
| | Bis-Ethylhexyloxyphenol | 1.00 | 2.00 | 3.00 | 4.00 | 5.00 | 6.00 | 7.00 | 2.00 | 4.00 |
| | Methoxyphenyl Triazine (Tinosorb S) | | | | | | | | | |
| Part B | Aqua | 5.00 | 5.00 | 5.00 | 5.00 | 5.00 | 5.00 | 5.00 | 5.00 | 5.00 |
| | Glycerin | 4.00 | 4.00 | 4.00 | 4.00 | 4.00 | 4.00 | 4.00 | 4.00 | 4.00 |
| | Galactoarabinan | 0.25 | 0.25 | 0.25 | 0.25 | 0.25 | 0.25 | 0.25 | 0.25 | 0.25 |
| | Disodium Ethylene | 1.00 | 1.00 | 1.00 | 1.00 | 1.00 | 1.00 | 1.00 | 1.00 | 1.00 |
| | Di(Cocamide PEG-15 Disulfate) | | | | | | | | | |
| | (and) Sodium Lauroyl | | | | | | | | | |
| | Lactylate | | | | | | | | | |
| Part C | Water | Qs to | Qs to | Qs to | Qs to | Qs to | Qs to | Qs to | Qs to | Qs to |
| | | 100 | 100 | 100 | 100 | 100 | 100 | 100 | 100 | 100 |
| Part D | Phenoxyethanol (and) Methyl- | 0.70 | 0.70 | 0.70 | 0.70 | 0.70 | 0.70 | 0.70 | 0.70 | 0.70 |
| | paraben (and) Ethylparaben | | | | | | | | | |
| | (and) Butylparaben (and) Propylparaben | | | | | | | | | |
| | (and) Isobutyl- | | | | | | | | | |
| | paraben | | | | | | | | | |
| | Alcohol Denatured | 10.00 | 10.00 | 10.00 | 10.00 | 10.00 | 10.00 | 10.00 | 10.00 | 10.00 |
| | Tocopheryl Acetate | 0.60 | 0.60 | 0.60 | 0.60 | 0.60 | 0.60 | 0.60 | 0.60 | 0.60 |

Example 18: Sun Spray Foaming

| | | A | B | C | D | E | F | G | H | I |
|---|---|---|---|---|---|---|---|---|---|---|
| | INCI-Name | Wt. % | Wt. % | Wt. % | Wt. % | Wt. % | Wt. % | Wt. % | Wt. % | Wt. % |
| Part A | Behenyl Alcohol (and) Glyceryl | 0.50 | 0.50 | 0.50 | 0.50 | 0.50 | 0.50 | 0.50 | 0.50 | 0.50 |
| | Stearate (and) Glyceryl Stearate | | | | | | | | | |
| | Citrate (and) Disodium | | | | | | | | | |
| | Ethylene Di(Cocamide PEG- | | | | | | | | | |
| | 15 Disulfate) | | | | | | | | | |
| | Butyl Methoxydibenzoyl- | 0.80 | 0.80 | 0.80 | 0.80 | 0.80 | 0.80 | 0.80 | 0.80 | 0.80 |
| | methane | | | | | | | | | |
| | Ethylhexyl Methoxycinnamate | 5.00 | 5.00 | 5.00 | 5.00 | 5.00 | 5.00 | 5.00 | 5.00 | 5.00 |
| | Isotrideceth-12 | 0.50 | 0.50 | 0.50 | 0.50 | 0.50 | 0.50 | 0.50 | 0.50 | 0.50 |
| | Ethylhexyl Salicylate | 4.00 | 4.00 | 4.00 | 4.00 | 4.00 | 4.00 | 4.00 | 4.00 | 4.00 |
| | Hydrogenated Coco- | 1.50 | 1.50 | 1.50 | 1.50 | 1.50 | 1.50 | 1.50 | 1.50 | 1.50 |
| | glycerides | | | | | | | | | |
| | Dimethyl capramide | 2.00 | 4.00 | 6.00 | 8.00 | 9.00 | 12.00 | 14.00 | 16.00 | 10.00 |
| | Bis-Ethylhexyloxyphenol Methoxyphenyl | 1.00 | 2.00 | 3.00 | 4.00 | 5.00 | 6.00 | 7.00 | 2.00 | 4.00 |
| | Triazine (Tinosorb S) | | | | | | | | | |
| Part B | C12-15 Alkyl Benzoate | 8.00 | 8.00 | 8.00 | 8.00 | 8.00 | 8.00 | 8.00 | 8.00 | 8.00 |
| | Aqua | 5.00 | 5.00 | 5.00 | 5.00 | 5.00 | 5.00 | 5.00 | 5.00 | 5.00 |
| | Glycerin | 4.00 | 4.00 | 4.00 | 4.00 | 4.00 | 4.00 | 4.00 | 4.00 | 4.00 |
| | Galactoarabinan | 0.25 | 0.25 | 0.25 | 0.25 | 0.25 | 0.25 | 0.25 | 0.25 | 0.25 |
| Part C | Disodium Ethylene Di(Cocamide | 1.00 | 1.00 | 1.00 | 1.00 | 1.00 | 1.00 | 1.00 | 1.00 | 1.00 |
| | PEG-15 Disulfate) (and) | | | | | | | | | |
| | Sodium Lauroyl Lactylate | | | | | | | | | |
| Part D | Water | Qs to | Qs to | Qs to | Qs to | Qs to | Qs to | Qs to | Qs to | Qs to |
| | | 100 | 100 | 100 | 100 | 100 | 100 | 100 | 100 | 100 |
| | Phenoxyethanol (and) Methyl- | 0.70 | 0.70 | 0.70 | 0.70 | 0.70 | 0.70 | 0.70 | 0.70 | 0.70 |
| | paraben (and) Ethylparaben | | | | | | | | | |
| | (and) Butylparaben (and) Propylparaben | | | | | | | | | |
| | (and) Isobutylparaben | | | | | | | | | |
| | Tocopheryl Acetate | 0.60 | 0.60 | 0.60 | 0.60 | 0.60 | 0.60 | 0.60 | 0.60 | 0.60 |

Example 19: Continuous Spray Fresh Cooling

| | A | B | C | D | E | F | G | H | I |
|---|---|---|---|---|---|---|---|---|---|
| | % | % | % | % | % | % | % | % | % |
| | b.w. | b.w. | b.w. | b.w. | b.w. | b.w. | b.w. | b.w. | b.w. |
| Emulsion | Vol. % | Vol. % | Vol. % | Vol. % | Vol. % | Vol. % | Vol. % | Vol. % | Vol. % |
| Butyl Methoxydibenzoyl- | 3.00 | 3.00 | 3.00 | 3.00 | 3.00 | 3.00 | 3.00 | 3.00 | 3.00 |
| methane | | | | | | | | | |
| Homosalate | 10.00 | 10.00 | 10.00 | 10.00 | 10.00 | 10.00 | 10.00 | 10.00 | 10.00 |
| Ethylhexyl Salicylate | 5.00 | 5.00 | 5.00 | 5.00 | 5.00 | 5.00 | 5.00 | 5.00 | 5.00 |
| Octocrylene | 2.35 | 2.35 | 2.35 | 2.35 | 2.35 | 2.35 | 2.35 | 2.35 | 2.35 |
| Benzophenone-3 | 2.00 | 2.00 | 2.00 | 2.00 | 2.00 | 2.00 | 2.00 | 2.00 | 2.00 |
| SD-Alcohol 40-2 | 30.00 | 30.00 | 30.00 | 30.00 | 30.00 | 30.00 | 30.00 | 30.00 | 30.00 |
| Diethylhexyl 2,6-Naphthalate | 6.00 | 6.00 | 6.00 | 6.00 | 6.00 | 6.00 | 6.00 | 6.00 | 6.00 |
| Trisiloxane (and) Dimethicone | 4.00 | 4.00 | 4.00 | 4.00 | 4.00 | 4.00 | 4.00 | 4.00 | 4.00 |
| Acrylates/Octylacrylamide Co- | 3.00 | 3.00 | 3.00 | 3.00 | 3.00 | 3.00 | 3.00 | 3.00 | 3.00 |
| polymer | | | | | | | | | |
| PPG-5-Ceteth-20 | 2.00 | 2.00 | 2.00 | 2.00 | 2.00 | 2.00 | 2.00 | 2.00 | 2.00 |
| Tocopheryl Acetate | 1.00 | 1.00 | 1.00 | 1.00 | 1.00 | 1.00 | 1.00 | 1.00 | 1.00 |
| Ascorbyl Palmitate | 0.3 | 0.3 | 0.3 | 0.3 | 0.3 | 0.3 | 0.3 | 0.3 | 0.3 |
| Retinyl Palmitate | 0.3 | 0.3 | 0.3 | 0.3 | 0.3 | 0.3 | 0.3 | 0.3 | 0.3 |
| Cyclopentasiloxane (and) Acrylates/ | 1.00 | 1.00 | 1.00 | 1.00 | 1.00 | 1.00 | 1.00 | 1.00 | 1.00 |
| Dimethicone Copolymer | | | | | | | | | |
| Propylene Glycol (and) Ethoxydiglycol | 1.50 | 1.50 | 1.50 | 1.50 | 1.50 | 1.50 | 1.50 | 1.50 | 1.50 |
| (and) Menthyl PCA | | | | | | | | | |
| (and) Lauryl PCA | | | | | | | | | |
| Dimethyl capramide | 2.00 | 4.00 | 6.00 | 8.00 | 9.00 | 12.00 | 14.00 | 16.00 | 10.00 |
| Bis-Ethylhexyloxyphenol Methoxyphenyl | 1.00 | 2.00 | 3.00 | 4.00 | 5.00 | 6.00 | 7.00 | 2.00 | 4.00 |
| Triazine (Tinosorb S) | | | | | | | | | |
| Parfume | q.s. | q.s. | q.s. | q.s. | q.s. | q.s. | q.s. | q.s. | q.s. |
| Aqua | ad 100 | ad 100 | ad 100 | ad 100 | ad 100 | ad 100 | ad 100 | ad 100 | ad 100 |
| Propellent | Qs | Qs | Qs | Qs | Qs | Qs | Qs | Qs | Qs |

Example 20: Continuous Spray Sun Protection

| | A | B | C | D | E | F | G | H | I |
|---|---|---|---|---|---|---|---|---|---|
| | % b.w. | % b.w. | % b.w. | % b.w. | % b.w. | % b.w. | % b.w. | % b.w. | % b.w. |
| Emulsion | Vol. % | Vol. % | Vol. % | Vol. % | Vol. % | Vol. % | Vol. % | Vol. % | Vol. % |
| Ethylhexyl Methoxy- | 7.50 | 7.50 | 7.50 | 7.50 | 7.50 | 7.50 | 7.50 | 7.50 | 7.50 |
| cinnamate | | | | | | | | | |
| Homosalate | 5.00 | 5.00 | 5.00 | 5.00 | 5.00 | 5.00 | 5.00 | 5.00 | 5.00 |
| Ethylhexyl Salicylate | 5.00 | 5.00 | 5.00 | 5.00 | 5.00 | 5.00 | 5.00 | 5.00 | 5.00 |
| Benzophenone-3 | 3.00 | 3.00 | 3.00 | 3.00 | 3.00 | 3.00 | 3.00 | 3.00 | 3.00 |
| SD-Alcohol 40-2 | 60.00 | 60.00 | 60.00 | 60.00 | 60.00 | 60.00 | 60.00 | 60.00 | 60.00 |
| | V/V | V/V | V/V | V/V | V/V | V/V | V/V | V/V | V/V |
| Dimethyl capramide | 2.00 | 4.00 | 6.00 | 8.00 | 9.00 | 12.00 | 14.00 | 16.00 | 10.00 |
| Bis-Ethylhexyloxyphenol | 1.00 | 2.00 | 3.00 | 4.00 | 5.00 | 6.00 | 7.00 | 2.00 | 4.00 |
| Methoxyphenyl | | | | | | | | | |
| Triazine (Tinosorb S) | | | | | | | | | |
| Acrylates/Octyl- | 3.00 | 3.00 | 3.00 | 3.00 | 3.00 | 3.00 | 3.00 | 3.00 | 3.00 |
| acrylamide Copolymer | | | | | | | | | |
| Parfume | q.s. | q.s. | q.s. | q.s. | q.s. | q.s. | q.s. | q.s. | q.s. |
| Propellent | Qs | Qs | Qs | Qs | Qs | Qs | Qs | Qs | Qs |

Example 21: Continuous Spray Kids

| | A | B | C | D | E | F | G | H | I |
|---|---|---|---|---|---|---|---|---|---|
| | % | % | % | % | % | % | % | % | % |
| | b.w. | b.w. | b.w. | b.w. | b.w. | b.w. | b.w. | b.w. | b.w. |
| Emulsion | Vol. % | Vol. % | Vol. % | Vol. % | Vol. % | Vol. % | Vol. % | Vol. % | Vol. % |
| Butyl Methoxydibenzoylmethane | 1.00 | 1.00 | 1.00 | 1.00 | 1.00 | 1.00 | 1.00 | 1.00 | 1.00 |
| Ethylhexyl Methoxycinnamate | 7.50 | 7.50 | 7.50 | 7.50 | 7.50 | 7.50 | 7.50 | 7.50 | 7.50 |
| Ethylhexyl Salicylate | 2.00 | 2.00 | 2.00 | 2.00 | 2.00 | 2.00 | 2.00 | 2.00 | 2.00 |
| Octocrylene | 0.80 | 0.80 | 0.80 | 0.80 | 0.80 | 0.80 | 0.80 | 0.80 | 0.80 |
| Benzophenone-3 | 2.00 | 2.00 | 2.00 | 2.00 | 2.00 | 2.00 | 2.00 | 2.00 | 2.00 |
| Acrylates/C12-22 Alkyl | 3.00 | 3.00 | 3.00 | 3.00 | 3.00 | 3.00 | 3.00 | 3.00 | 3.00 |
| Methacrylate Copolymer | | | | | | | | | |
| Octyldodecyl Neopentanoate | 3.00 | 3.00 | 3.00 | 3.00 | 3.00 | 3.00 | 3.00 | 3.00 | 3.00 |
| Ceteareth-20 | 1.00 | 1.00 | 1.00 | 1.00 | 1.00 | 1.00 | 1.00 | 1.00 | 1.00 |
| Titanium Dioxide particles | 3.00 | 3.00 | 3.00 | 3.00 | 3.00 | 3.00 | 3.00 | 3.00 | 3.00 |
| size 10 to 100 nm | | | | | | | | | |
| Caprylyl Glycol | 1.00 | 1.00 | 1.00 | 1.00 | 1.00 | 1.00 | 1.00 | 1.00 | 1.00 |
| Aloe Barbadensis Leaf | 1.00 | 1.00 | 1.00 | 1.00 | 1.00 | 1.00 | 1.00 | 1.00 | 1.00 |
| Extract (Aloe Vera) | | | | | | | | | |
| Tocopheryl Acetate | 0.70 | 0.70 | 0.70 | 0.70 | 0.70 | 0.70 | 0.70 | 0.70 | 0.70 |
| Dimethyl capramide | 2.00 | 4.00 | 6.00 | 8.00 | 9.00 | 12.00 | 14.00 | 16.00 | 10.00 |
| Bis-Ethylhexyloxyphenol | 1.00 | 2.00 | 3.00 | 4.00 | 5.00 | 6.00 | 7.00 | 2.00 | 4.00 |
| Methoxyphenyl Triazine | | | | | | | | | |
| (Tinosorb S) | | | | | | | | | |
| Microcrystalline Cellulose | 0.30 | 0.30 | 0.30 | 0.30 | 0.30 | 0.30 | 0.30 | 0.30 | 0.30 |
| (and) Cellulose Gum | | | | | | | | | |
| Disodium Ethylene | 2.00 | 2.00 | 2.00 | 2.00 | 2.00 | 2.00 | 2.00 | 2.00 | 2.00 |
| Di(Cocamide PEG-15 | | | | | | | | | |
| Disulfate) (and) Sodium | | | | | | | | | |
| Lauroyl Lactylate | | | | | | | | | |
| Behenyl Alcohol (and) | 0.50 | 0.50 | 0.50 | 0.50 | 0.50 | 0.50 | 0.50 | 0.50 | 0.50 |
| Glyceryl Stearate (and) | | | | | | | | | |
| Glyceryl Stearate Citrate | | | | | | | | | |
| (and) Disodium Ethylene | | | | | | | | | |
| Di(Cocamide PEG-15 | | | | | | | | | |
| Disulfate) | | | | | | | | | |
| Acacia Gum | 0.20 | 0.20 | 0.20 | 0.20 | 0.20 | 0.20 | 0.20 | 0.20 | 0.20 |
| Triethanolamine | 0.15 | 0.15 | 0.15 | 0.15 | 0.15 | 0.15 | 0.15 | 0.15 | 0.15 |
| Methyldibromo Glutaronitrile | 0.5 | 0.5 | 0.5 | 0.5 | 0.5 | 0.5 | 0.5 | 0.5 | 0.5 |
| (and) Methyl- | | | | | | | | | |
| chloroisothiazolinone | | | | | | | | | |
| (and) Methylisothiazolinone | | | | | | | | | |
| (and) Phenoxy- | | | | | | | | | |
| ethanol | | | | | | | | | |
| Acrylates/C10-30 Alkyl | 0.15 | 0.15 | 0.15 | 0.15 | 0.15 | 0.15 | 0.15 | 0.15 | 0.15 |
| Acrylate Crosspolymer | | | | | | | | | |
| Disodium EDTA | 0.10 | 0.10 | 0.10 | 0.10 | 0.10 | 0.10 | 0.10 | 0.10 | 0.10 |
| BHT | 0.05 | 0.05 | 0.05 | 0.05 | 0.05 | 0.05 | 0.05 | 0.05 | 0.05 |
| Parfume | q.s. | q.s. | q.s. | q.s. | q.s. | q.s. | q.s. | q.s. | q.s. |
| Water dest. | ad 100 | ad 100 | ad 100 | ad 100 | ad 100 | ad 100 | ad 100 | ad 100 | ad 100 |
| Propellent | Qs | Qs | Qs | Qs | Qs | Qs | Qs | Qs | Qs |

Example 22: Lait écran extrême peaux sensibles

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Ethylhexyl Methoxycinnamate | 9.00 | 9.00 | 9.00 | 9.00 | 9.00 | 9.00 | 9.00 | 9.00 | 9.00 |
| | C12-15 Alkyl Benzoate | 3.50 | 3.50 | 3.50 | 3.50 | 3.50 | 3.50 | 3.50 | 3.50 | 3.50 |
| | Cyclopentasiloxane | 9.00 | 9.00 | 9.00 | 9.00 | 9.00 | 9.00 | 9.00 | 9.00 | 9.00 |
| | (and) Cyclohexasiloxane | | | | | | | | | |
| | Polyglyceryl-4 Diiso- | | 3.00 | | | 3.00 | | | | 2.00 |
| | stearate polyhydroxystearate/ | | | | | | | | | |
| | Sebacate | | | | | | | | | |
| | Sorbitan sesquioleate | | | 3.00 | | | 3.00 | | 1.50 | |
| | Trioleate sorbitan | | | | 3.00 | | | 3.00 | | 1.50 |
| | PEG-30 Dipolyhydroxystearate | 3.00 | 1.00 | 1.00 | 1.00 | 1.00 | 1.00 | 1.00 | 1.00 | 1.00 |
| | PEG-45/Dodecyl Glycol | 1.00 | 1.00 | 1.00 | 1.00 | 1.00 | 1.00 | 1.00 | 1.00 | 1.00 |
| | Copolymer | | | | | | | | | |
| | Hydrogenated Castor Oil | 1.50 | 1.50 | 1.50 | 1.50 | 1.50 | 1.50 | 1.50 | 1.50 | 1.50 |
| | Tribehenin | 0.40 | 0.40 | 0.40 | 0.40 | 0.40 | 0.40 | 0.40 | 0.40 | 0.40 |
| | Disteardimonium Hectorite | 0.60 | 0.60 | 0.60 | 0.60 | 0.60 | 0.60 | 0.60 | 0.60 | 0.60 |
| | Titanium Dioxide particles | 3.50 | 3.50 | 3.50 | 3.50 | 3.50 | 3.50 | 3.50 | 3.50 | 3.50 |
| | size 10 to 100 nm | | | | | | | | | |
| | Zinc Oxide | 0.50 | 0.50 | 0.50 | 0.50 | 0.50 | 0.50 | 0.50 | 0.50 | 0.50 |
| | Dimethyl capramide | 2.00 | 4.00 | 6.00 | 8.00 | 9.00 | 12.00 | 14.00 | 16.00 | 10.00 |
| | Bis-Ethylhexyloxyphenol | 1.00 | 2.00 | 3.00 | 4.00 | 5.00 | 6.00 | 7.00 | 2.00 | 4.00 |
| | Methoxyphenyl Triazine | | | | | | | | | |
| | (Tinosorb S) | | | | | | | | | |
| Part B | AQUA | Qs | Qs | Qs | Qs | Qs | Qs | Qs | Qs | Qs |
| | Glycerin | 2.00 | 2.00 | 2.00 | 2.00 | 2.00 | 2.00 | 2.00 | 2.00 | 2.00 |
| Part C | Methylene Bis-Benzotriazolyl | 10.00 | 10.00 | 10.00 | 10.00 | 10.00 | 10.00 | 10.00 | 10.00 | 10.00 |
| | Tetramethylbutyl- | | | | | | | | | |
| | phenol (and) Aqua (and) | | | | | | | | | |
| | Decyl Glucoside (and) | | | | | | | | | |
| | Propylene Glycol (and) | | | | | | | | | |
| | Xanthan Gum | | | | | | | | | |
| Part D | Phenoxyethanol (and) | 0.70 | 0.70 | 0.70 | 0.70 | 0.70 | 0.70 | 0.70 | 0.70 | 0.70 |
| | Methylparaben (and) | | | | | | | | | |
| | Ethylparaben (and) Butylparaben | | | | | | | | | |
| | (and) Propyl- | | | | | | | | | |
| | paraben (and) Isobutyl- | | | | | | | | | |
| | paraben | | | | | | | | | |
| | Tocopheryl Acetate | 0.50 | 0.50 | 0.50 | 0.50 | 0.50 | 0.50 | 0.50 | 0.50 | 0.50 |
| | Silica Dimethyl Silylate | 0.60 | 0.60 | 0.60 | 0.60 | 0.60 | 0.60 | 0.60 | 0.60 | 0.60 |

Example 23: Sun Milk W/O

| | | A | B | C | D | E | F | G | H | I |
|---|---|---|---|---|---|---|---|---|---|---|
| | | % | % | % | % | % | % | % | % | % |
| | INCI-Name | w/w | w/w | w/w | w/w | w/w | w/w | w/w | w/w | w/w |
| Part A | Butylen Glycol Dicaprylate/ | 5.50 | 5.50 | 5.50 | 5.50 | 5.50 | 5.50 | 5.50 | 5.50 | 5.50 |
| | Dicaprate | | | | | | | | | |
| | Ethylhexyl Methoxy- | 7.50 | 7.50 | 7.50 | 7.50 | 7.50 | 7.50 | 7.50 | 7.50 | 7.50 |
| | cinnamate | | | | | | | | | |
| | Butyl Methoxydibenzoylmethane | 3.50 | 3.50 | 3.50 | 3.50 | 3.50 | 3.50 | 3.50 | 3.50 | 3.50 |
| | Octocrylene | 2.50 | 2.50 | 2.50 | 2.50 | 2.50 | 2.50 | 2.50 | 2.50 | 2.50 |
| | C18-36 Acid Triglyceride | 0.50 | 0.50 | 0.50 | 0.50 | 0.50 | 0.50 | 0.50 | 0.50 | 0.50 |
| | Isopropyl Stearate | 3.50 | 3.50 | 3.50 | 3.50 | 3.50 | 3.50 | 3.50 | 3.50 | 3.50 |
| | Polyglyceryl-4 Diisostearate | | 3.00 | | | 3.00 | | | | 2.00 |
| | polyhydroxystearate/ | | | | | | | | | |
| | Sebacate | | | | | | | | | |
| | Sorbitan sesquioleate | | | 3.00 | | | 3.00 | | 1.50 | |
| | Trioleate sorbitan | | | | 3.00 | | | 3.00 | | 1.50 |
| | Titanium Dioxide particles | 3.70 | 3.70 | 3.70 | 3.70 | 3.70 | 3.70 | 3.70 | 3.70 | 3.70 |
| | size 10 to 100 nm | | | | | | | | | |
| | Polyglyceryl-2 Dipolyhydroxystearate | 5.00 | | | | | | | | |
| | Diethylhexyl Butamido | 0.50 | 0.50 | 0.50 | 0.50 | 0.50 | 0.50 | 0.50 | 0.50 | 0.50 |
| | Triazone | | | | | | | | | |
| | Cetyl Dimethicone | 0.50 | 0.50 | 0.50 | 0.50 | 0.50 | 0.50 | 0.50 | 0.50 | 0.50 |
| | Dimethyl capramide | 2.00 | 4.00 | 6.00 | 8.00 | 9.00 | 12.00 | 14.00 | 16.00 | 10.00 |
| | Bis-Ethylhexyloxyphenol | 1.00 | 2.00 | 3.00 | 4.00 | 5.00 | 6.00 | 7.00 | 2.00 | 4.00 |
| | Methoxyphenyl Triazine | | | | | | | | | |
| | (Tinosorb S) | | | | | | | | | |
| Part B | Aqua | Qs | Qs | Qs | Qs | Qs | Qs | Qs | Qs | Qs |
| | Butylene Glycol | 3.50 | 3.50 | 3.50 | 3.50 | 3.50 | 3.50 | 3.50 | 3.50 | 3.50 |
| | Aluminum Starch Octenylsuccinate | 0.40 | 0.40 | 0.40 | 0.40 | 0.40 | 0.40 | 0.40 | 0.40 | 0.40 |
| | Magnesium Sulfate Heptahydrate | 0.30 | 0.30 | 0.30 | 0.30 | 0.30 | 0.30 | 0.30 | 0.30 | 0.30 |
| | Trisodium EDTA | 0.10 | 0.10 | 0.10 | 0.10 | 0.10 | 0.10 | 0.10 | 0.10 | 0.10 |
| Part C | Alcohol Denatured | 2.00 | 2.00 | 2.00 | 2.00 | 2.00 | 2.00 | 2.00 | 2.00 | 2.00 |
| Part D | BHT | 0.05 | 0.05 | 0.05 | 0.05 | 0.05 | 0.05 | 0.05 | 0.05 | 0.05 |
| | Phenoxyethanol (and) | 0.50 | 0.50 | 0.50 | 0.50 | 0.50 | 0.50 | 0.50 | 0.50 | 0.50 |
| | Methylparaben (and) Ethylparaben | | | | | | | | | |
| | (and) Butylparaben | | | | | | | | | |
| | (and) Propylparaben | | | | | | | | | |
| | (and) Isobutylparaben | | | | | | | | | |
| | Tocopheryl Acetate | 1.00 | 1.00 | 1.00 | 1.00 | 1.00 | 1.00 | 1.00 | 1.00 | 1.00 |

Example 24: W/Si Sun Cream

| | | A | B | C | D | E | F | G | H | I |
|---|---|---|---|---|---|---|---|---|---|---|
| | INCI-Name | % | % | % | % | % | % | % | % | % |
| Part A | Cyclopentasiloxane | 3.00 | 3.00 | 3.00 | 3.00 | 3.00 | 3.00 | 3.00 | 3.00 | 3.00 |
| | (and) Dimethicone/Vinyl | | | | | | | | | |
| | Dimethicone Crosspolymer | | | | | | | | | |
| | PEG-10 Dimethicone | 0.30 | 0.30 | 0.30 | 0.30 | 0.30 | 0.30 | 0.30 | 0.30 | 0.30 |
| | Dimethicone (and) Dimethicone/ | 4.00 | 4.00 | 4.00 | 4.00 | 4.00 | 4.00 | 4.00 | 4.00 | 4.00 |
| | PEG-10/15 | | | | | | | | | |
| | Crosspolymer | | | | | | | | | |
| | Dimethicone | 10.70 | 10.70 | 10.70 | 10.70 | 10.70 | 10.70 | 10.70 | 10.70 | 10.70 |
| | Dimethyl capramide | 2.00 | 4.00 | 6.00 | 8.00 | 9.00 | 12.00 | 14.00 | 16.00 | 10.00 |
| | Bis-Ethylhexyloxyphenol | 1.00 | 2.00 | 3.00 | 4.00 | 5.00 | 6.00 | 7.00 | 2.00 | 4.00 |
| | Methoxyphenyl Triazine | | | | | | | | | |
| | (Tinosorb S) | | | | | | | | | |
| Part B | 1.3-Butylen Glycol | 8.00 | 8.00 | 8.00 | 8.00 | 8.00 | 8.00 | 8.00 | 8.00 | 8.00 |
| | Sodium Citrate | 0.20 | 0.20 | 0.20 | 0.20 | 0.20 | 0.20 | 0.20 | 0.20 | 0.20 |
| | Ethyl Alcohol | 5.00 | 5.00 | 5.00 | 5.00 | 5.00 | 5.00 | 5.00 | 5.00 | 5.00 |
| | Sodium Chloride | 0.50 | 0.50 | 0.50 | 0.50 | 0.50 | 0.50 | 0.50 | 0.50 | 0.50 |
| | Aqua | Qs to | Qs to | Qs to | Qs to | Qs to | Qs to | Qs to | Qs to | Qs to |
| | | 100 | 100 | 100 | 100 | 100 | 100 | 100 | 100 | 100 |

Example 25: W/Si Sun Cream

| | | A | B | C | D | E | F | G | H | I |
|---|---|---|---|---|---|---|---|---|---|---|
| | INCI-Name | % | % | % | % | % | % | % | % | % |
| Part A | Cyclopentasiloxane | 10.00 | 10.00 | 10.00 | 10.00 | 10.00 | 10.00 | 10.00 | 10.00 | 10.00 |
| | (and) PEG/PPG-18/18 | | | | | | | | | |
| | Dimethicone | | | | | | | | | |
| | Cyclomethicone | 9.00 | 9.00 | 9.00 | 9.00 | 9.00 | 9.00 | 9.00 | 9.00 | 9.00 |
| | Cyclomethicone (and) | 3.50 | 3.50 | 3.50 | 3.50 | 3.50 | 3.50 | 3.50 | 3.50 | 3.50 |
| | Trimethylsiloxysilicate | | | | | | | | | |
| | Cyclopentasiloxane | 7.00 | 7.00 | 7.00 | 7.00 | 7.00 | 7.00 | 7.00 | 7.00 | 7.00 |
| | (and) Dimethicone | | | | | | | | | |
| | Crosspolymer | | | | | | | | | |
| | Dimethyl capramide | 2.00 | 4.00 | 6.00 | 8.00 | 9.00 | 12.00 | 14.00 | 16.00 | 10.00 |
| | Bis-Ethylhexyloxyphenol | 1.00 | 2.00 | 3.00 | 4.00 | 5.00 | 6.00 | 7.00 | 2.00 | 4.00 |
| | Methoxyphenyl Triazine | | | | | | | | | |
| | (Tinosorb S) | | | | | | | | | |
| Part B | Divinyldimethicone/Di- | 2.30 | 2.30 | 2.30 | 2.30 | 2.30 | 2.30 | 2.30 | 2.30 | 2.30 |
| | methicone Copolymer | | | | | | | | | |
| | (and) C12-13 Pareth-23 | | | | | | | | | |
| | (and) C12-13 Pareth-3 | | | | | | | | | |
| | Sodium Chloride | 2.00 | 2.00 | 2.00 | 2.00 | 2.00 | 2.00 | 2.00 | 2.00 | 2.00 |
| | Aqua | Qs to | Qs to | Qs to | Qs to | Qs to | Qs to | Qs to | Qs to | Qs to |
| | | 100 | 100 | 100 | 100 | 100 | 100 | 100 | 100 | 100 |

Example 26: W/Si Sun Cream

| | | A | B | C | D | E | F | G | H | I |
|---|---|---|---|---|---|---|---|---|---|---|
| | INCI-Name | % | % | % | % | % | % | % | % | % |
| Part A | Bis-PEG/PPG-14/14 Di- | 2.00 | 2.00 | 2.00 | 2.00 | 2.00 | 2.00 | 2.00 | 2.00 | 2.00 |
| | methicone (and) Cyclo- | | | | | | | | | |
| | pentasiloxane | | | | | | | | | |
| | Cetyl PEG/PPG-10/1 | 1.70 | 1.70 | 1.70 | 1.70 | 1.70 | 1.70 | 1.70 | 1.70 | 1.70 |
| | Dimethicone | | | | | | | | | |
| | Cyclopentasiloxane | 13.00 | 13.00 | 13.00 | 13.00 | 13.00 | 13.00 | 13.00 | 13.00 | 13.00 |
| | (and) Cyclohexasiloxane | | | | | | | | | |
| | Ethylhexyl Palmitate | 1.50 | 1.50 | 1.50 | 1.50 | 1.50 | 1.50 | 1.50 | 1.50 | 1.50 |
| | Cetyl Dimethicone | 1.00 | 1.00 | 1.00 | 1.00 | 1.00 | 1.00 | 1.00 | 1.00 | 1.00 |
| | Ethylhexyl methoxycinnamate | 3.00 | 3.00 | 3.00 | 3.00 | 3.00 | 3.00 | 3.00 | 3.00 | 3.00 |
| | Ethylhexyl Salicylate | 3.00 | 3.00 | 3.00 | 3.00 | 3.00 | 3.00 | 3.00 | 3.00 | 3.00 |
| | Dimethyl capramide | 2.00 | 4.00 | 6.00 | 8.00 | 9.00 | 12.00 | 14.00 | 16.00 | 10.00 |
| | Bis-Ethylhexyloxyphenol | 1.00 | 2.00 | 3.00 | 4.00 | 5.00 | 6.00 | 7.00 | 2.00 | 4.00 |
| | Methoxyphenyl Triazine | | | | | | | | | |
| | (Tinosorb S) | | | | | | | | | |
| Part B | Propylene Glycol | 2.00 | 2.00 | 2.00 | 2.00 | 2.00 | 2.00 | 2.00 | 2.00 | 2.00 |
| | Magnesium Sulfate Heptahydrate | 2.50 | 2.50 | 2.50 | 2.50 | 2.50 | 2.50 | 2.50 | 2.50 | 2.50 |
| | Glycerin | 2.00 | 2.00 | 2.00 | 2.00 | 2.00 | 2.00 | 2.00 | 2.00 | 2.00 |
| | Alcohol Denatured | 1.00 | 1.00 | 1.00 | 1.00 | 1.00 | 1.00 | 1.00 | 1.00 | 1.00 |
| | Aqua | Qs to | Qs to | Qs to | Qs to | Qs to | Qs to | Qs to | Qs to | Qs to |
| | | 100 | 100 | 100 | 100 | 100 | 100 | 100 | 100 | 100 |

Example 27: Lipstick Composition

| INCI-Name | A | B | C | D | E | F | G | H | I |
|---|---|---|---|---|---|---|---|---|---|
| Ingredients | % | % | % | % | % | % | % | % | % |
| Isoeicosane | 10.40 | | 13.00 | | | | | | |
| Isododecane | | | | | | | | 5.00 | 5.00 |
| polyisobutene | 7.60 | | 12.00 | | | | | | |
| polybutene | | 8.00 | | | | | | | |
| Tridecyl Trimellitate | 14.55 | 10.00 | 17.40 | | | | | | |
| Diisostearate Dimerate | | | | | | | | 15.00 | 15.00 |
| Octyl palmitate | 10.00 | | 15.00 | | | | | | |
| Caprylic/Capric Triglycerides | | | | | 12.00 | 20.00 | | | |
| PEG-4 Diheptanoate | 7.60 | | | | | | | | |
| Polyglyceryl-3 | | | | | | | | 3.00 | 3.00 |
| Diisostearate | | | | | | | | | |
| Castor (Ricinus communis) | 4.00 | | | | | | | | |
| oil (and) colorants | | | | | | | | | |
| Castor (Ricinus communis) | | | 4.00 | 50.00 | 22.00 | 25.00 | | | |
| oil | | | | | | | | | |
| Trihydroxystearin | | 1.00 | | | | | | | |
| Ozokerite | | | | | 4.50 | 1.50 | | | |
| Otyldodecanol (and) Quaternium- | | 10.00 | | | | | | | |
| 18 Hectorite (and) | | | | | | | | | |
| Propylene carbonate | | | | | | | | | |
| Synthetic wax | | | | | | | | 10.10 | 9.40 |
| Microcrystalline wax | | | | | 2.00 | | | | |
| Bees wax | 12.25 | | 7.00 | | | | 25.00 | | |
| Candellila wax | 5.70 | | 7.50 | 15.00 | 7.00 | 8.00 | | | |
| Carnauba wax | 12.40 | | 8.00 | 5.00 | 3.00 | 3.50 | | | |
| Cera Alba | | 5.00 | | | | | | | |
| Ceresine wax | | 5.00 | | | | 5.00 | | | |
| Tribehenin | | 12.00 | | | | | | | |
| Octyldodecanol | | 24.00 | 10.50 | | 17.00 | | 54.00 | | |
| polyethylene | 1.50 | | | | | | | | |
| Calcium Aluminum Boro- | | | 1.00 | | | | | | |
| silicate Cetearyl Alcohol | | 10.00 | | | | | | | |
| Paraffin | | | | | | | | 7.60 | 7.60 |
| Acetylated Lanolin Alcohol | | | | 5.00 | 5.00 | 10.00 | | | |
| Dimethyl capramide | 2.00 | 4.00 | 6.00 | 8.00 | 9.00 | 12.00 | 14.00 | 16.00 | 10.00 |
| Bis-Ethylhexyloxyphenol | 1.00 | 2.00 | 3.00 | 4.00 | 5.00 | 6.00 | 7.00 | 2.00 | 4.00 |
| Methoxyphenyl Triazine | | | | | | | | | |
| (Tinosorb S) | | | | | | | | | |
| Pigments/pearls | | | | | | | | 13.00 | 13.00 |
| Colorants | | | | 12.00 | 13.00 | 13.00 | 9.50 | | |
| Tocopherol Acetate | | 0.40 | 0.30 | | | | | 0.20 | 0.20 |
| Cyclomethicone | | | | | | | | 21.00 | 32.70 |
| Preservative | | 0.10 | 0.30 | | | | | 0.10 | 0.10 |
| Butyl Methoxydibenzoylmethane | 1.00 | 1.50 | 2.00 | 1.00 | 1.50 | 2.00 | 0.50 | 2.00 | 2.00 |
| Ethylhexyl Triazone | 3.00 | 3.00 | 2.00 | 2.00 | 3.00 | 2.00 | 1.00 | 2.00 | 2.00 |

Example 28: Sunscreen Oleogel

| | | A | B | C | D | E | F | G | H | I |
|---|---|---|---|---|---|---|---|---|---|---|
| Part | Ingredient | Wt. % | Wt. % | Wt. % | Wt. % | Wt. % | Wt. % | Wt. % | Wt. % | Wt. % |
| | Isopropyl Myristate | 38.00 | 38.00 | 38.00 | 38.00 | 38.00 | 38.00 | 33.00 | 23.00 | 13.00 |
| | C12-15 Alkyl Benzoate | 10.00 | 10.00 | 10.00 | 10.00 | 10.00 | 10.00 | 10.00 | 10.00 | 10.00 |
| | Caprylic/Capric Triglyceride | 25.00 | 25.00 | 20.00 | 15.00 | 10.00 | 5.00 | 5.00 | 5.00 | 5.00 |
| | Perfume | q.s. | q.s. | q.s. | q.s. | q.s. | q.s. | q.s. | q.s. | q.s. |
| | Disteardimonium Hectorite | 15.00 | 15.00 | 15.00 | 15.00 | 15.00 | 15.00 | 15.00 | 15.00 | 15.00 |
| | Dimethyl capramide | 2.00 | 4.00 | 6.00 | 8.00 | 9.00 | 12.00 | 14.00 | 16.00 | 10.00 |
| | Bis-Ethylhexyloxyphenol | 1.00 | 2.00 | 3.00 | 4.00 | 5.00 | 6.00 | 7.00 | 2.00 | 4.00 |
| | Methoxyphenyl Triazine | | | | | | | | | |
| | (Tinosorb S) | | | | | | | | | |
| | Propylene Carbonate | 5.00 | 5.00 | 5.00 | 5.00 | 5.00 | 5.00 | 5.00 | 5.00 | 5.00 |

Example 29: Sunscreen Oleogel

| | A | B | C | D | E | F | G | H | I |
|---|---|---|---|---|---|---|---|---|---|
| Ingredient | Wt. % | Wt. % | Wt. % | Wt. % | Wt. % | Wt. % | Wt. % | Wt. % | Wt. % |
| Prunus Amygdalus Dulcis | 23.00 | 21.00 | 15.00 | 10.00 | 5.00 | 5.00 | 5.00 | 5.00 | 5.00 |
| (Sweet Almond) Oil | | | | | | | | | |
| Polyglyceryl-3 Beeswax | 14.00 | 14.00 | 14.00 | 14.00 | 14.00 | 9.00 | 4.00 | 4.00 | 4.00 |
| Isopropyl Palmitate | 15.00 | 15.00 | 15.00 | 15.00 | 15.00 | 15.00 | 15.00 | 5.00 | 5.00 |
| Simmondsia Chinensis (Jojoba) | 12.00 | 12.00 | 12.00 | 12.00 | 12.00 | 12.00 | 12.00 | 12.00 | 2.00 |
| Seed Oil | | | | | | | | | |
| Sesamum Indicum (Sesame) | 9.00 | 9.00 | 9.00 | 9.00 | 9.00 | 9.00 | 9.00 | 9.00 | 9.00 |
| Seed Oil | | | | | | | | | |
| C12-15 Alkyl Benzoate | 10.00 | 10.00 | 10.00 | 10.00 | 10.00 | 10.00 | 10.00 | 10.00 | 10.00 |
| Persea Gratissima (Avocado) | 9.00 | 9.00 | 9.00 | 9.00 | 9.00 | 9.00 | 9.00 | 9.00 | 9.00 |
| Oil | | | | | | | | | |
| Cetyl Stearyl Alcohol | 4.00 | 4.00 | 4.00 | 4.00 | 4.00 | 4.00 | 4.00 | 4.00 | 4.00 |
| Ozokerite | 1.00 | 1.00 | 1.00 | 1.00 | 1.00 | 1.00 | 1.00 | 1.00 | 1.00 |
| Copernicia Cerifera (Carnauba) | 0.80 | 0.80 | 0.80 | 0.80 | 0.80 | 0.80 | 0.80 | 0.80 | 0.80 |
| Wax | | | | | | | | | |
| Retinyl Palmitate | 0.10 | 0.10 | 0.10 | 0.10 | 0.10 | 0.10 | 0.10 | 0.10 | 0.10 |
| Dimethyl capramide | 2.00 | 4.00 | 6.00 | 8.00 | 9.00 | 12.00 | 14.00 | 16.00 | 10.00 |
| Bis-Ethylhexyloxyphenol Methoxyphenyl | 1.00 | 2.00 | 3.00 | 4.00 | 5.00 | 6.00 | 7.00 | 2.00 | 4.00 |
| Triazine (Tinosorb | | | | | | | | | |
| S) | | | | | | | | | |
| Tocopheryl Acetate | 0.10 | 0.10 | 0.10 | 0.10 | 0.10 | 0.10 | 0.10 | 0.10 | 0.10 |

Example 30: O/W/O Soft Cream

| | | A | B | C | D | E | F | G | H | I |
|---|---|---|---|---|---|---|---|---|---|---|
| Part | Ingredient | Wt. % | Wt. % | Wt. % | Wt. % | Wt. % | Wt. % | Wt. % | Wt. % | Wt. % |
| A | PEG-60 Hydrogenated | 0.40 | 0.40 | 0.40 | 0.40 | 0.40 | 0.40 | 0.40 | 0.40 | 0.40 |
| | Castor Oil | | | | | | | | | |
| A | Water | 0.90 | 0.90 | 0.90 | 0.90 | 0.90 | 0.90 | 0.90 | 0.90 | 0.90 |
| B | Tocopheryl Acetate | 2.00 | 2.00 | 2.00 | 2.00 | 2.00 | 2.00 | 2.00 | 2.00 | 2.00 |
| B | Retinyl Palmitate | 0.50 | 0.50 | 0.50 | 0.50 | 0.50 | 0.50 | 0.50 | 0.50 | 0.50 |
| B | Caprylic/Capric Triglyceride | 2.50 | 2.50 | 2.50 | 2.50 | 2.50 | 2.50 | 2.50 | 2.50 | 2.50 |
| B | Dimethyl capramide | 2.00 | 4.00 | 6.00 | 8.00 | 9.00 | 12.00 | 14.00 | 16.00 | 10.00 |
| B | Bis-Ethylhexyloxyphenol | 1.00 | 2.00 | 3.00 | 4.00 | 5.00 | 6.00 | 7.00 | 2.00 | 4.00 |
| | Methoxyphenyl Triazine | | | | | | | | | |
| | (Tinosorb S) | | | | | | | | | |
| C | Water | q.s. | q.s. | q.s. | q.s. | q.s. | q.s. | q.s. | q.s. | q.s. |
| C | Sodium Chloride | 0.40 | 0.40 | 0.40 | 0.40 | 0.40 | 0.40 | 0.40 | 0.40 | 0.40 |
| D | Cetyl PEG/PPG-10/1 | 2.00 | 2.00 | 2.00 | 2.00 | 2.00 | 2.00 | 2.00 | 2.00 | 2.00 |
| | Dimethicone | | | | | | | | | |
| D | Microcrystalline Wax | 0.90 | 0.90 | 0.90 | 0.90 | 0.90 | 0.90 | 0.90 | 0.90 | 0.90 |
| D | Hydrogenated Castor Oil | 0.60 | 0.60 | 0.60 | 0.60 | 0.60 | 0.60 | 0.60 | 0.60 | 0.60 |
| D | Decyl Oleate | 10.50 | 10.50 | 10.50 | 10.50 | 10.50 | 10.50 | 10.50 | 10.50 | 10.50 |
| D | Caprylic/Capric Triglyceride | 10.00 | 10.00 | 10.00 | 10.00 | 10.00 | 10.00 | 10.00 | 10.00 | 10.00 |
| D | Jojoba (Buxus Chinensis) | 2.00 | 2.00 | 2.00 | 2.00 | 2.00 | 2.00 | 2.00 | 2.00 | 2.00 |
| | Oil | | | | | | | | | |
| D | Preservative, Parfum | q.s. | q.s. | q.s. | q.s. | q.s. | q.s. | q.s. | q.s. | q.s. |

Example 31: W/O/W Multiple Emulsion

| | | A | B | C | D | E | F | G | H | I |
|---|---|---|---|---|---|---|---|---|---|---|
| Part | Ingredient | Wt. % | Wt. % | Wt. % | Wt. % | Wt. % | Wt. % | Wt. % | Wt. % | Wt. % |
| | I. Primary emulsion W/O: | | | | | | | | | |
| A | Isohexadecane | 15.00 | 15.00 | 15.00 | 15.00 | 15.00 | 15.00 | 15.00 | 15.00 | 15.00 |
| A | Caprylic/capric triglycerides | 7.50 | 7.50 | 7.50 | 7.50 | 7.50 | 7.50 | 7.50 | 7.50 | 7.50 |
| A | PPG-15 stearyl ether | 7.50 | 7.50 | 7.50 | 7.50 | 7.50 | 7.50 | 7.50 | 7.50 | 7.50 |
| A | PEG-30 Dipolyhydroxystearate | 4.00 | 4.00 | 4.00 | 4.00 | 4.00 | 4.00 | 4.00 | 4.00 | 4.00 |
| A | Dimethyl capramide | 2.00 | 4.00 | 6.00 | 8.00 | 9.00 | 12.00 | 14.00 | 16.00 | 10.00 |
| A | Bis-Ethylhexyloxyphenol | 1.00 | 2.00 | 3.00 | 4.00 | 5.00 | 6.00 | 7.00 | 2.00 | 4.00 |
| | Methoxyphenyl Triazine | | | | | | | | | |
| | (Tinosorb S) | | | | | | | | | |
| B | Water | q.s. 100% | q.s. 100% | q.s. 100% | q.s. 100% | q.s. 100% | q.s. 100% | q.s. 100% | q.s. 100% | q.s. 100% |
| B | Sodium chloride | 0.40 | 0.40 | 0.40 | 0.40 | 0.40 | 0.40 | 0.40 | 0.40 | 0.40 |
| B | Preservative | q.s. | q.s. | q.s. | q.s. | q.s. | q.s. | q.s. | q.s. | q.s. |
| | II. Secondary emulsion | | | | | | | | | |
| | W/O/W: | | | | | | | | | |
| A & B | Primary emulsion (W/O) | 70.00 | 70.00 | 70.00 | 70.00 | 70.00 | 70.00 | 70.00 | 70.00 | 70.00 |
| C | Water | q.s. 100% | q.s. 100% | q.s. 100% | q.s. 100% | q.s. 100% | q.s. 100% | q.s. 100% | q.s. 100% | q.s. 100% |
| C | Poloxamer 407 | 2.00 | 2.00 | 2.00 | 2.00 | 2.00 | 2.00 | 2.00 | 2.00 | 2.00 |
| D | Carbomer | 0.50 | 0.50 | 0.50 | 0.50 | 0.50 | 0.50 | 0.50 | 0.50 | 0.50 |
| E | Sodium chloride | 0.20 | 0.20 | 0.20 | 0.20 | 0.20 | 0.20 | 0.20 | 0.20 | 0.20 |
| F | Preservative | q.s. | q.s. | q.s. | q.s. | q.s. | q.s. | q.s. | q.s. | q.s. |
| G | Triethanolamine | 0.50 | 0.50 | 0.50 | 0.50 | 0.50 | 0.50 | 0.50 | 0.50 | 0.50 |

Example 32: Conventional Powder

| | A | B | C | D | E | F | G | H | I |
|---|---|---|---|---|---|---|---|---|---|
| Ingredients | Wt. % | Wt. % | Wt. % | Wt. % | Wt. % | Wt. % | Wt. % | Wt. % | Wt. % |
| Kaolin | | | 7.00 | | 10.00 | 20.00 | | | |
| Mica | | | | | | | | 56.7 | |
| Talc | q.s. 100% | q.s. 100% | q.s. 100% | q.s. 100% | q.s. 100% | q.s. 100% | q.s. 100% | q.s. 100% | q.s. 100% |
| Titanium Dioxide particles | 5.00 | 9.00 | 10.00 | 8.00 | 5.00 | 3.00 | | | 5.00 |
| size 10 to 100 nm | | | | | | | | | |
| Precipitated Chalk | 8.00 | 10.00 | 3.00 | | | | | | 8.00 |
| Bismuth oxychloride | | | | | | | | 10.0 | |
| Magnesium Carbonate | | | 2.00 | 3.00 | 2.00 | 2.00 | | | |
| Zinc Stearate | 7.00 | 6.00 | 5.00 | 5.00 | | | | | 7.00 |
| Dimethyl capramide | 2.00 | 4.00 | 6.00 | 8.00 | 9.00 | 12.00 | 14.00 | 16.00 | 10.00 |
| Bis-Ethylhexyloxyphenol | 1.00 | 2.00 | 3.00 | 4.00 | 5.00 | 6.00 | 7.00 | 2.00 | 4.00 |
| Methoxyphenyl Triazine | | | | | | | | | |
| (Tinosorb S) | | | | | | | | | |
| Fumed Silica | | | | | 15.00 | 15.00 | | | |
| Color (and) Fragrance | qs | qs | qs | qs | qs | qs | | qs | qs |
| Ethylhexyl Methoxycinnamate | 0.7 | 0.50 | 0.80 | 0.70 | 0.50 | 0.80 | | 0.30 | 0.7 |
| Ethylhexyl Salicylate | 4.80 | 4.50 | 6.20 | | | | | | 4.80 |
| Octocrylene | | | | 4.30 | 3.50 | 6.20 | | 3.00 | |

Example 33: Cream-to-powder

| | A | B | C | D | E | F | G | H | I |
|---|---|---|---|---|---|---|---|---|---|
| Ingredients | Wt. % | Wt. % | Wt. % | Wt. % | Wt. % | Wt. % | Wt. % | Wt. % | Wt. % |
| Isoeicosane | 10.00 | 26.90 | | 5.00 | 20.90 | | 5.00 | 28.90 | |
| Polyisobutene | 2.00 | | | 2.00 | | | 2.00 | | |
| Squalene | | | 10.00 | | | 10.00 | | | 5.00 |
| Mineral oil | | | 3.00 | | | 3.00 | | | 3.00 |
| Cetearyl Octanoate | 20.50 | | | 20.50 | | | 20.50 | | |
| Octyl Hydroxystearate | | | 7.00 | | | 7.00 | | | 7.00 |
| Diemthicone | | | 17.00 | | | 10.00 | | | 7.00 |
| Pentaerythrityl Hydrogenated | | 12.00 | | | 12.00 | | | 12.00 | |
| Rosinate | | | | | | | | | |
| Oleyl alcohol | 5.00 | | | 5.00 | | | 5.00 | | |
| Lecithin (and) Polysorbate- | | 0.20 | | | 0.20 | | | 0.20 | |
| 20 (and) | | | | | | | | | |
| Sorbitan Laurate | | | | | | | | | |
| (and) Propylene Glycol | | | | | | | | | |
| Stearate (and) | | | | | | | | | |
| Propylene Glycol | | | | | | | | | |
| Laurate | | | | | | | | | |
| Polyglyceryl-3 | | | 3.00 | | | 3.00 | | | 3.00 |
| Diisostearate | | | | | | | | | |
| Ceresine wax | 4.50 | | | 4.50 | | | 4.50 | | |
| Carnauba wax | | 4.00 | | | 4.00 | | | 4.00 | |
| Microcrystalline wax | | | 7.00 | | | 7.00 | | | 7.00 |
| Mica | | 14.00 | 18.20 | | 14.00 | 18.20 | | 4.00 | 11.20 |
| Talc | 11.60 | | | 11.60 | | | 11.60 | | |
| Talc (and) Ethylene | | | 8.00 | | | 8.00 | | | 8.00 |
| methacrylate co- | | | | | | | | | |
| polymer (and) Iso- | | | | | | | | | |
| propyl Titanium Tri- | | | | | | | | | |
| isostearate | | | | | | | | | |
| Titanium Dioxide | 15.00 | 7.00 | | 15.00 | 7.00 | | 10.00 | 7.00 | |
| particles size 10 to | | | | | | | | | |
| 100 nm | | | | | | | | | |
| Titanium Dioxide | | | 16.00 | | | 16.00 | | | 16.00 |
| particles size 10 to | | | | | | | | | |
| 100 nm | | | | | | | | | |
| Polyethylene | 4.00 | | | 4.00 | | | 4.00 | | |
| Mica (and) Silica | | 3.00 | | | 3.00 | | | 3.00 | |
| Dimethyl capramide | 2.00 | 4.00 | 6.00 | 8.00 | 9.00 | 12.00 | 14.00 | 16.00 | 10.00 |
| Bis- | 1.00 | 2.00 | 3.00 | 4.00 | 5.00 | 6.00 | 7.00 | 2.00 | 4.00 |
| Ethylhexyloxyphenol | | | | | | | | | |
| Methoxyphenyl Triazine | | | | | | | | | |
| (Tinosorb S) | | | | | | | | | |
| Silica | 17.75 | | | 17.75 | | | 12.75 | | |
| Calcium Aluminum | 1.00 | | | 1.00 | | | 1.00 | | |
| Borosilicate (and) | | | | | | | | | |
| Bismuth oxychloride | | | | | | | | | |
| Calcium Aluminum | | 10.00 | | | 10.00 | | | 5.00 | |
| Borosilicate | | | | | | | | | |
| Bismuth oxychloride | | 10.00 | | | 10.00 | | | 5.00 | |
| (and) Silica (and) | | | | | | | | | |
| Mica | | | | | | | | | |
| Iron Oxides | 3.40 | 1.70 | | 3.40 | 1.70 | | 3.40 | 1.70 | |
| Tocopherol Acetate | 0.25 | | | 0.25 | | | 0.25 | | |
| Preservative | q.s | 0.20 | | | 0.20 | | | 0.20 | |
| Ethylhexyl Triazone | 0.50 | 1.00 | 0.60 | 0.50 | 1.00 | 0.60 | 0.50 | 1.00 | 0.60 |
| Methoxycinnamates | 4.50 | 8.00 | 7.20 | 4.50 | 8.00 | 7.20 | 4.50 | 8.00 | 7.20 |
| UV absorbers | | | | | | | | | |

Example 34: Pressed Powder

| | A | B | C | D | E | F | G | H | I |
|---|---|---|---|---|---|---|---|---|---|
| Ingredients | Wt. % | Wt. % | Wt. % | Wt. % | Wt. % | Wt. % | Wt. % | Wt. % | Wt. % |
| Isoeicosane | | | | | | 5.00 | | | |
| Squalane | | | 5.00 | 1.80 | | | | | 5.00 |
| Trioctanoin | | | 1.50 | | | | | | 1.50 |
| PEG-4 Diheptanoate | | 2.00 | | | 2.00 | | | 2.00 | |
| Pentaerythritol Tetraoctanoate | | | | 1.20 | | | | | |
| Petrolatum | 1.00 | | 1.00 | | | | 1.00 | | 1.00 |
| Dimethicone (and) | | | | 5.00 | | | | | |
| Trimethyl Siloxy Silicate | | | | | | | | | |
| Synthetic wax | | | 1.00 | | | | | | 1.00 |
| Dimethyl capramide | 2.00 | 4.00 | 6.00 | 8.00 | 9.00 | 12.00 | 14.00 | 16.00 | 10.00 |
| Bis-Ethylhexyloxyphenol | 1.00 | 2.00 | 3.00 | 4.00 | 5.00 | 6.00 | 7.00 | 2.00 | 4.00 |
| Methoxyphenyl Triazine (Tinosorb | | | | | | | | | |
| S) | | | | | | | | | |
| Zinc Stearate | | | | | | 4.00 | | | |
| Mica | 34.50 | | | 57.00 | | | 24.50 | | |
| Mica (and) Methicone | | | | | 10.00 | | | | |
| Mica dimethicone coated | | | 40.00 | | | | | | 20.00 |
| Talc (and) Methicone | | 54.00 | | | 32.00 | 10.00 | | q.s. 100% | |
| Talc dimethicone coated | | | qsp | | | | | | qsp |
| Talc | 35.00 | | | | | q.s 100% | q.s 100% | | |
| Silica | | | | | | 2.50 | | | |
| Titanium Dioxide particles | 10.00 | 4.00 | | | 4.00 | 10.70 | 10.00 | 4.00 | |
| size 10 to 100 nm | | | | | | | | | |
| PTFE | 2.00 | 10.00 | 10.00 | | 10.00 | | 2.00 | 10.00 | 10.00 |
| Polyethylene | 8.15 | 15.00 | | | 16.00 | | | 15.00 | |
| Iron oxides (and) Silica | | | | | | 2.95 | | | |
| Ethylene acrylate acid co- | | | | 18.00 | | | | | |
| polymer | | | | | | | | | |
| Iron Oxides | 3.35 | 7.00 | q.s | | 7.00 | 0.25 | 3.35 | 7.00 | q.s |
| Iron oxides (and) Isopropyl | | | | 2.00 | | | | | |
| Titanium Triisostearate | | | | | | | | | |
| Dimethicone/Vinyl dimethicone | | | 8.00 | | | | | | 8.00 |
| Crosspolymer | | | | | | | | | |
| Preservative | | 0.50 | | | 0.50 | 0.50 | | 0.50 | |
| Methylene Bis-Benzotriazolyl | | 3.00 | 6.00 | 4.00 | 5.00 | | | 3.00 | 6.00 |
| Tetramethylbutyl- | | | | | | | | | |
| phenol | | | | | | | | | |
| Benzophenones UV absorbers | 5.00 | 2.50 | 4.00 | 3.00 | 3.50 | 3.00 | 5.00 | 2.50 | 4.00 |

Example 35: Foundations: Anhydrous forms

| | A | B | C | D | E | F | G | H | I |
|---|---|---|---|---|---|---|---|---|---|
| Ingredients | Wt. % | Wt. % | Wt. % | Wt. % | Wt. % | Wt. % | Wt. % | Wt. % | Wt. % |
| Tribehenin | | | | 3.00 | | | | 3.00 | 3.00 |
| Isoeicosane | | 11.50 | | 15.00 | | 1.50 | | 5.00 | 5.00 |
| Isododecane | | | | 17.50 | | | | 7.50 | 7.50 |
| Isododecane (and) Quaternium- | | | | 24.5 | | | | 20.00 | 20.0 |
| 18 Hectorite | | | | | | | | | |
| (and) Propylene carbonate | | | | | | | | | |
| Polyisobutene | | 8.2 | | | | 2.50 | | | |
| Isononyl Isononanoate | qsp | | 15.10 | | qsp | | 15.10 | | |
| Cetearyl Octanoate | | 23.20 | | | | 13.20 | | | |
| Sorbitan Sesquioleate | 0.50 | | | | 0.50 | | | | |
| Sorbitan Trioleate | | | 0.20 | | | | 0.20 | | |
| Polyglyceryl-4 | | 0.80 | | | | 0.80 | | | |
| Isostearate (and) Cetyl | | | | | | | | | |
| Dimethicone | | | | | | | | | |
| Polyglyceryl-4 | | | | 1.00 | | | | 1.00 | 1.00 |
| Isostearate (and) Cetyl | | | | | | | | | |
| Dimethicone copolyol | | | | | | | | | |
| (and) Hexyl Laurate | | | | | | | | | |
| Dimethiconol Beeswax | | | 5.00 | | | | 5.00 | | |
| Stearyl dimethicone | | | 10.00 | | | | 10.00 | | |
| Oleyl Alcohol | | 4.00 | | | | 4.00 | | | |
| Cyclopentasiloxane | 15.00 | | | | 15.00 | | | | |
| Cylopentasiloxane (and) | 15.00 | | | | 15.00 | | | | |
| Quaternium-18 Hectorite | | | | | | | | | |
| Polyethylene | | 4.50 | | | | 4.50 | | | |
| Microcrystalline wax | | | 3.00 | | | | 3.00 | | |
| Ceresin wax | | 5.50 | | | | 5.50 | | | |
| Carnauba Wax | | | 4.00 | | | | 4.00 | | |
| Sericite | | | 12.00 | | | | 2.00 | | |
| Talc | 5.00 | 7.75 | | | 5.00 | 7.75 | | | |
| Titanium Dioxide particles | 15.00 | 12.00 | 7.00 | 12.00 | 15.00 | 7.00 | 7.00 | 20.0 | 17.0 |
| size 10 to 100 nm | | | | | | | | | |
| Dimethyl capramide | 2.00 | 4.00 | 6.00 | 8.00 | 9.00 | 12.00 | 14.00 | 16.00 | 10.00 |
| Bis-Ethylhexyloxyphenol | 1.00 | 2.00 | 3.00 | 4.00 | 5.00 | 6.00 | 7.00 | 2.00 | 4.00 |
| Methoxyphenyl Triazine | | | | | | | | | |
| (Tinosorb S) | | | | | | | | | |
| Spherical Silica | | 16.00 | 26.00 | 3.00 | | 16.00 | 16.00 | 3.00 | 3.00 |
| Iron oxides | 2.50 | 4.25 | 1.70 | 1.25 | 2.50 | 4.25 | 1.70 | 1.20 | 1.20 |
| Bismuth Oxychloride | | | | 3.80 | | | | 3.80 | 3.80 |
| PVP/eicosane copolymer | 1.00 | | | | 1.00 | | | | |
| Tocopherol Acetate | 0.50 | | | 0.50 | 0.50 | | | 0.50 | 0.50 |
| Ethylhexyl Triazone | 0.80 | 3.00 | 2.00 | 1.00 | 0.80 | 3.00 | 2.00 | 1.00 | 1.00 |
| Benzophenone UV absorbers | 3.00 | 5.00 | 4.00 | 2.00 | 3.00 | 5.00 | 4.00 | 2.00 | 2.00 |

Example 36: Foundations; O/W forms

| | A | B | C | D | E | F | G | H | I |
|---|---|---|---|---|---|---|---|---|---|
| Ingredients | Wt. % | Wt. % | Wt. % | Wt. % | Wt. % | Wt. % | Wt. % | Wt. % | Wt. % |
| Butylene Glycol | 6.50 | 6.50 | 6.50 | 6.50 | 6.50 | 6.50 | 6.50 | 6.50 | 6.50 |
| Magnesium Aluminum | 0.50 | 0.50 | 0.50 | 0.50 | 0.50 | 0.50 | 0.50 | 0.50 | 0.50 |
| Silicate | | | | | | | | | |
| Sodium Carboxymethyl- | 0.30 | 0.30 | 0.30 | 0.30 | 0.30 | 0.30 | 0.30 | 0.30 | 0.30 |
| cellulose | | | | | | | | | |
| Xanthan Gum | 0.10 | 0.10 | 0.10 | 0.10 | 0.10 | 0.10 | 0.10 | 0.10 | 0.10 |
| Triethanolamine | 1.00 | 1.00 | 1.00 | 1.00 | 1.00 | 1.00 | 1.00 | 1.00 | 1.00 |
| Steareth-21 | | 0.30 | | 0.30 | | 0.30 | | 0.30 | 0.30 |
| Steareth-2 | | 0.70 | | 0.70 | | 0.70 | | 0.70 | 0.70 |
| Polysorbate 20 | 0.25 | | 0.25 | | 0.25 | | 0.25 | | |
| Sericite | 7.00 | 6.00 | 7.00 | 6.00 | 7.00 | 6.00 | 7.00 | 6.00 | 6.00 |
| Titanium Dioxide particles | 7.00 | 7.00 | 7.00 | 7.00 | 7.00 | 7.00 | 7.00 | 7.00 | 7.00 |
| size 10 to 100 nm | | | | | | | | | |
| Iron oxides | 1.10 | 1.00 | 1.10 | 1.00 | 1.10 | 1.00 | 1.10 | 1.00 | 1.00 |
| PMMA | | 3.00 | | 3.00 | | 3.00 | | 3.00 | 3.00 |
| Spherical Silica | 1.00 | | 1.00 | | 1.00 | | 1.00 | | |
| Cetearyl octanoate | 10.00 | | 10.00 | | 10.00 | | 10.00 | | |
| Stearyl Dimethicone | | 4.00 | | 4.00 | | 4.00 | | 4.00 | 4.00 |
| Dimethicone | | 2.00 | | 2.00 | | 2.00 | | 2.00 | 2.00 |
| Stearic Acid | 2.50 | 1.00 | 2.50 | 1.00 | 2.50 | 1.00 | 2.50 | 1.00 | 1.00 |
| Isostearic Acid | | 1.00 | | 1.00 | | 1.00 | | 1.00 | 1.00 |
| Isononyl isononanoate | | 4.00 | | 4.00 | | 4.00 | | 4.00 | 4.00 |
| Glyceryl stearate | 2.00 | | 2.00 | | 2.00 | | 2.00 | | |
| Glyceryl monostearate | | 1.50 | | 1.50 | | 1.50 | | 1.50 | 1.50 |
| Tridecyl trimellilate | 2.00 | | 2.00 | | 2.00 | | 2.00 | | |
| Dimethyl capramide | 2.00 | 4.00 | 6.00 | 8.00 | 9.00 | 12.00 | 14.00 | 16.00 | 10.00 |
| Bis-Ethylhexyloxyphenol | 1.00 | 2.00 | 3.00 | 4.00 | 5.00 | 6.00 | 7.00 | 2.00 | 4.00 |
| Methoxyphenyl Triazine | | | | | | | | | |
| (Tinosorb S) | | | | | | | | | |
| Water | qsp | qsp | qsp | qsp | qsp | qsp | qsp | qsp | qsp |
| Preservative | 0.55 | 0.55 | 0.55 | 0.55 | 0.55 | 0.55 | 0.55 | 0.55 | 0.55 |
| Methylene Bis-Benzotriazolyl | 5.00 | 6.00 | 5.00 | 6.00 | 5.00 | 6.00 | 5.00 | 6.00 | 6.00 |
| Tetramethylbutyl- | | | | | | | | | |
| phenol (and) Aqua (and) | | | | | | | | | |
| Decyl Glucoside (and) Propylene | | | | | | | | | |
| Glycol (and) Xanthan | | | | | | | | | |
| Gum | | | | | | | | | |

Example 37: Hydrous and Anhydrous Gels

| | A | B | C | D | E | F | G | H | I |
|---|---|---|---|---|---|---|---|---|---|
| Ingredients | Wt. % | Wt. % | Wt. % | Wt. % | Wt. % | Wt. % | Wt. % | Wt. % | Wt. % |
| Isoeicosane | 43.05 | | 2.00 | 43.85 | | 2.00 | 43.85 | | 2.00 |
| Polyisobutene | | | 2.00 | | | 2.00 | | | 2.00 |
| Zinc Laurate | | | 4.00 | | | 4.00 | | | 4.00 |
| Sorbitan Isostearate | | | 0.50 | | | 0.50 | | | 0.50 |
| Synthetic wax | | 5.00 | | | 5.00 | | | 5.00 | |
| Carnauba wax | 3.00 | | | 3.00 | | | 3.00 | | |
| Isododecane (and) Quaternium- | 6.00 | | | 6.00 | | | 6.00 | | |
| 18 Hectorite | | | | | | | | | |
| (and) Propylene Carbonate | | | | | | | | | |
| Dipentaerythrityl Hexacaprylate/ | 1.00 | | | 1.00 | | | 1.00 | | |
| Hexacaprate | | | | | | | | | |
| Mica | q.s | q.s | q.s 100% | q.s 100% | q.s 100% | q.s 100% | q.s 100% | q.s 100% | q.s 100% |
| | 100% | 100% | | | | | | | |
| Talc | | | 20.0 | | | 20.0 | | | 20.0 |
| Titanium Dioxide particles | | 2.00 | | | 2.00 | | | 2.00 | |
| size 10 to 100 nm | | | | | | | | | |
| PTFE | | | 2.00 | | | 2.00 | | | 2.00 |
| Silica | | | 0.50 | | | 0.50 | | | 0.50 |
| Calcium Aluminum Boro- | | 2.00 | | | 2.00 | | | 2.00 | |
| silicate | | | | | | | | | |
| Dimethyl capramide | 2.00 | 4.00 | 6.00 | 8.00 | 9.00 | 12.00 | 14.00 | 16.00 | 10.00 |
| Bis-Ethylhexyloxyphenol | 1.00 | 2.00 | 3.00 | 4.00 | 5.00 | 6.00 | 7.00 | 2.00 | 4.00 |
| Methoxyphenyl Triazine (Tinosorb | | | | | | | | | |
| S) | | | | | | | | | |
| Bismuth oxychloride | 10.00 | | 4.00 | 10.00 | | 4.00 | 10.00 | | 4.00 |
| Iron Oxides | 5.75 | 6.00 | 6.00 | 5.75 | 6.00 | 6.00 | 5.75 | 6.00 | 6.00 |
| Preservative | 0.20 | 0.25 | 0.45 | 0.20 | 0.25 | 0.45 | 0.20 | 0.25 | 0.45 |
| Ethylhexyl Triazone | 1.00 | 2.00 | 3.00 | 1.00 | 2.00 | 3.00 | 1.00 | 2.00 | 3.00 |
| Micronized ZnO | 4.00 | 2.00 | 3.00 | 4.00 | 2.00 | 3.00 | 4.00 | 2.00 | 3.00 |

Example 38: Microemulsion

| | | A | B | C | D | E | F | G | H | I |
|---|---|---|---|---|---|---|---|---|---|---|
| Part | Ingredient | Wt. % | Wt. % | Wt. % | Wt. % | Wt. % | Wt. % | Wt. % | Wt. % | Wt. % |
| A | Glyceryl Cocoate/Citrate/Lactate | 30.00 | 30.00 | 30.00 | 30.00 | 30.00 | 30.00 | 30.00 | 30.0 | 30.0 |
| A | C12-15 Alkyl Benzoate | 10.00 | 10.00 | 10.00 | 10.00 | 10.00 | 10.00 | 10.00 | 10.0 | 10.0 |
| A | Caprylic/Capric Triglyceride | 20.00 | 20.00 | 20.00 | 20.00 | 20.00 | 20.00 | 20.00 | 20.0 | 20.0 |
| A | PEG-6 Caprylic/Capric Glycerides | 20.00 | 20.00 | 20.00 | 20.00 | 20.00 | 20.00 | 20.00 | 20.0 | 10.0 |
| A | Preservative | q.s. | q.s. | q.s. | q.s. | q.s. | q.s. | q.s. | q.s. | q.s. |
| | Dimethyl capramide | 2.00 | 4.00 | 6.00 | 8.00 | 9.00 | 12.00 | 14.00 | 16.00 | 10.00 |
| | Bis-Ethylhexyloxyphenol | 1.00 | 2.00 | 3.00 | 4.00 | 5.00 | 6.00 | 7.00 | 2.00 | 4.00 |
| | Methoxyphenyl | | | | | | | | | |
| | Triazine (Tinosorb S) | | | | | | | | | |
| A | Water | Qs to 100 | Qs to 100 | Qs to 100 | Qs to 100 | Qs to 100 | Qs to 100 | Qs to 100 | Qs to 100 | Qs to 100 |
| B | Fragrance | q.s. | q.s. | q.s. | q.s. | q.s. | q.s. | q.s. | q.s. | q.s. |

Example 39: Microemulsion

| | | A | B | C | D | E | F | G | H | I |
|---|---|---|---|---|---|---|---|---|---|---|
| Part | Ingredient | Wt. % | Wt. % | Wt. % | Wt. % | Wt. % | Wt. % | Wt. % | Wt. % | Wt. % |
| A | Caprylic/Capric Triglyceride | 15.00 | 15.00 | 15.00 | 15.00 | 15.00 | 15.00 | 15.00 | 15.0 | 15.0 |
| A | C12-15 Alkyl Benzoate | 10.00 | 10.00 | 10.00 | 10.00 | 10.00 | 10.00 | 10.00 | 10.0 | 10.0 |
| A | PEG-6 Caprylic/Capric Glycerides | 27.00 | 27.00 | 27.00 | 27.00 | 27.00 | 27.00 | 27.00 | 27.0 | 27.0 |
| A | Glyceryl Cocoate/Citrate Lactate | 28.00 | 28.00 | 28.00 | 28.00 | 28.00 | 28.00 | 28.00 | 28.0 | 28.0 |
| | Dimethyl capramide | 2.00 | 4.00 | 6.00 | 8.00 | 9.00 | 12.00 | 14.00 | 16.00 | 10.00 |
| | Bis-Ethylhexyloxyphenol | 1.00 | 2.00 | 3.00 | 4.00 | 5.00 | 6.00 | 7.00 | 2.00 | 4.00 |
| | Methoxyphenyl | | | | | | | | | |
| | Triazine (Tinosorb S) | | | | | | | | | |
| A | Water | Qs to 100 | Qs to 100 | Qs to 100 | Qs to 100 | Qs to 100 | Qs to 100 | Qs to 100 | Qs to 100 | Qs to 100 |

Example 40: Cationic O/W Sun Cream

| | | A | B | C | D | E | F | G | H | I |
|---|---|---|---|---|---|---|---|---|---|---|
| | INCI-Name | % | % | % | % | % | % | % | % | % |
| Part | Distearyldimonium | 3.50 | 3.50 | 3.50 | 3.50 | 3.50 | 3.50 | 3.50 | 3.50 | 3.50 |
| A | Chloride | | | | | | | | | |
| | Glyceryl Stearate | 1.50 | 1.50 | 1.50 | 1.50 | 1.50 | 1.50 | 1.50 | 1.50 | 1.50 |
| | Stearyl Alcohol | 1.00 | 1.00 | 1.00 | 1.00 | 1.00 | 1.00 | 1.00 | 1.00 | 1.00 |
| | C12-15 Alkyl | 2.00 | 2.00 | 2.00 | 2.00 | 2.00 | 2.00 | 2.00 | 2.00 | 2.00 |
| | Benzoate | | | | | | | | | |
| | Diethylhexyl | 3.00 | 3.00 | 3.00 | 3.00 | 3.00 | 3.00 | 3.00 | 3.00 | 3.00 |
| | Carbonate | | | | | | | | | |
| | Triisostearin | 1.00 | 1.00 | 1.00 | 1.00 | 1.00 | 1.00 | 1.00 | 1.00 | 1.00 |
| | Butyl | 2.00 | 2.00 | 2.00 | 2.00 | 2.00 | 2.00 | 2.00 | 2.00 | 2.00 |
| | Methoxydibenzoyl- | | | | | | | | | |
| | methane | | | | | | | | | |
| | Octocrylene | 7.00 | 7.00 | 7.00 | 7.00 | 7.00 | 7.00 | 7.00 | 7.00 | 7.00 |
| | Ethylhexyl | 1.50 | 1.50 | 1.50 | 1.50 | 1.50 | 1.50 | 1.50 | 1.50 | 1.50 |
| | Methoxycinnamate | | | | | | | | | |
| | Dimethyl capramide | 2.00 | 4.00 | 6.00 | 8.00 | 9.00 | 12.00 | 14.00 | 16.00 | 10.00 |
| | Bis- | 1.00 | 2.00 | 3.00 | 4.00 | 5.00 | 6.00 | 7.00 | 2.00 | 4.00 |
| | Ethylhexyloxyphenol | | | | | | | | | |
| | Methoxyphenyl | | | | | | | | | |
| | Triazine (Tinosorb S) | | | | | | | | | |
| Part | Aqua | Qs to 100 | Qs to 100 | Qs to 100 | Qs to 100 | Qs to 100 | Qs to 100 | Qs to 100 | Qs to 100 | Qs to 100 |
| B | Glycerin | 2.00 | 2.00 | 2.00 | 2.00 | 2.00 | 2.00 | 2.00 | 2.00 | 2.00 |
| | Trisodium EDTA | 0.20 | 0.20 | 0.20 | 0.20 | 0.20 | 0.20 | 0.20 | 0.20 | 0.20 |
| Part | Phenoxyethanol (and) | 0.70 | 0.70 | 0.70 | 0.70 | 0.70 | 0.70 | 0.70 | 0.70 | 0.70 |
| C | Methylparaben | | | | | | | | | |
| | (and) Ethylparaben | | | | | | | | | |
| | (and) Butylparaben | | | | | | | | | |
| | (and) Propylparaben | | | | | | | | | |
| | (and) Isobutyl- | | | | | | | | | |
| | paraben | | | | | | | | | |
| | Tocopheryl Acetate | 0.80 | 0.80 | 0.80 | 0.80 | 0.80 | 0.80 | 0.80 | 0.80 | 0.80 |

Example 41: Cationic O/W Sun Cream

| | | A | B | C | D | E | F | G | H | I |
|---|---|---|---|---|---|---|---|---|---|---|
| | INCI-Name | % | % | % | % | % | % | % | % | % |
| Part A | Palmitamidopropyltrimonium | 6.00 | 6.00 | 6.00 | 6.00 | 6.00 | 6.00 | 6.00 | 6.00 | 6.00 |
| | Chloride | | | | | | | | | |
| | Stearyl Alcohol | 2.00 | 2.00 | 2.00 | 2.00 | 2.00 | 2.00 | 2.00 | 2.00 | 2.00 |
| | Isocetyl Palmitate | 4.00 | 4.00 | 4.00 | 4.00 | 4.00 | 4.00 | 4.00 | 4.00 | 4.00 |
| | Decyl Cocoate | 3.00 | 3.00 | 3.00 | 3.00 | 3.00 | 3.00 | 3.00 | 3.00 | 3.00 |
| | C12-15 Alkyl Benzoate | 2.00 | 2.00 | 2.00 | 2.00 | 2.00 | 2.00 | 2.00 | 2.00 | 2.00 |
| | Cetyl Dimethicone | 0.75 | 0.75 | 0.75 | 0.75 | 0.75 | 0.75 | 0.75 | 0.75 | 0.75 |
| | Titanium Dioxide particles size | 2.00 | 2.00 | 2.00 | 2.00 | 2.00 | 2.00 | 2.00 | 2.00 | 2.00 |
| | 10 to 100 nm | | | | | | | | | |
| | Butyl Methoxydibenzoyl- | 1.00 | 1.00 | 1.00 | 1.00 | 1.00 | 1.00 | 1.00 | 1.00 | 1.00 |
| | methane | | | | | | | | | |
| | Octocrylene | 6.00 | 6.00 | 6.00 | 6.00 | 6.00 | 6.00 | 6.00 | 6.00 | 6.00 |
| | Polysilicone-15 | 0.70 | 0.70 | 0.70 | 0.70 | 0.70 | 0.70 | 0.70 | 0.70 | 0.70 |
| | Dimethyl capramide | 2.00 | 4.00 | 6.00 | 8.00 | 9.00 | 12.00 | 14.00 | 16.00 | 10.00 |
| | Bis-Ethylhexyloxyphenol Methoxyphenyl | 1.00 | 2.00 | 3.00 | 4.00 | 5.00 | 6.00 | 7.00 | 2.00 | 4.00 |
| | Triazine (Tinosorb S) | | | | | | | | | |
| Part B | Aqua | Qs | Qs | Qs | Qs | Qs to 100 | Qs to 100 | Qs to 100 | Qs to 100 | Qs to 100 |
| | | to 100 | to 100 | to 100 | to 100 | | | | | |
| | Glycerin | 2.00 | 2.00 | 2.00 | 2.00 | 2.00 | 2.00 | 2.00 | 2.00 | 2.00 |
| | Trisodium EDTA | 0.10 | 0.10 | 0.10 | 0.10 | 0.10 | 0.10 | 0.10 | 0.10 | 0.10 |
| Part C | Capryl/Capramidopropyl Betaine | 8.00 | 8.00 | 8.00 | 8.00 | 8.00 | 8.00 | 8.00 | 8.00 | 8.00 |
| Part D | Phenoxyethanol (and) Methylparaben | 0.70 | 0.70 | 0.70 | 0.70 | 0.70 | 0.70 | 0.70 | 0.70 | 0.70 |
| | (and) Ethylparaben | | | | | | | | | |
| | (and) Butylparaben (and) Propylparaben | | | | | | | | | |
| | (and) Isobutylparaben | | | | | | | | | |
| | Tocopheryl Acetate | 0.80 | 0.80 | 0.80 | 0.80 | 0.80 | 0.80 | 0.80 | 0.80 | 0.80 |

Exmple 42: Si/W Sun Cream

| | | A | B | C | D | E | F | G | H | I |
|---|---|---|---|---|---|---|---|---|---|---|
| | INCI-Name | % | % | % | % | % | % | % | % | % |
| Part A | Cyclopentasiloxane (and) Dimethicone/ | 8.00 | 8.00 | 8.00 | 8.00 | 8.00 | 8.00 | 8.00 | 8.00 | 8.00 |
| | Vinyl Dimethicone | | | | | | | | | |
| | Crosspolymer | | | | | | | | | |
| | Dimethicone (and) Dimethicone/ | 30.00 | 30.00 | 30.00 | 30.00 | 30.00 | 30.00 | 30.00 | 30.00 | 10.00 |
| | Vinyl Dimethicone | | | | | | | | | |
| | Crosspolymer | | | | | | | | | |
| | Cyclopentasiloxane | 10.00 | 10.00 | 10.00 | 10.00 | 10.00 | 10.00 | 10.00 | 10.00 | 5.00 |
| | Dimethyl capramide | 2.00 | 4.00 | 6.00 | 8.00 | 9.00 | 12.00 | 14.00 | 16.00 | 10.00 |
| | Bis-Ethylhexyloxyphenol Methoxyphenyl | 1.00 | 2.00 | 3.00 | 4.00 | 5.00 | 6.00 | 7.00 | 2.00 | 4.00 |
| | Triazine (Tinosorb | | | | | | | | | |
| | S) | | | | | | | | | |
| Part B | 1.3-Butylen Glycol | 3.00 | 3.00 | 3.00 | 3.00 | 3.00 | 3.00 | 3.00 | 3.00 | 3.00 |
| | Polyglyceryl-3 Disiloxane Dimethicone | 0.60 | 0.60 | 0.60 | 0.60 | 0.60 | 0.60 | 0.60 | 0.60 | 0.60 |
| | Polyglyceryl-3 Polydimethylsiloxyethyl | 0.30 | 0.30 | 0.30 | 0.30 | 0.30 | 0.30 | 0.30 | 0.30 | 0.30 |
| | Dimethicone | | | | | | | | | |
| | Acrylamide/Sodium Acryloyldimethyltaurate | 0.60 | 0.60 | 0.60 | 0.60 | 0.60 | 0.60 | 0.60 | 0.60 | 0.60 |
| | Copolymer | | | | | | | | | |
| | (and) Isohexadecane (and) | | | | | | | | | |
| | Polysorbate 80 | | | | | | | | | |
| | Ammonium Acryloyldimethyltaurate/ | 0.65 | 0.65 | 0.65 | 0.65 | 0.65 | 0.65 | 0.65 | 0.65 | 0.65 |
| | VP Copolymer | | | | | | | | | |
| | Sodium Chloride | 0.10 | 0.10 | 0.10 | 0.10 | 0.10 | 0.10 | 0.10 | 0.10 | 0.10 |
| | Aqua | Qs | Qs | Qs | Qs | Qs | Qs to 100 | Qs to 100 | Qs to 100 | Qs to 100 |
| | | to 100 | to 100 | to 100 | to 100 | to 100 | | | | |

Example 43: Sunscreen Emulsion

| | | A | B | C | D | E |
|---|---|---|---|---|---|---|
| | INCI-Name | % w/w | % w/w | % w/w | % w/w | % w/w |
| Part A | Glyceryl stearate | 1.80 | 1.80 | 1.80 | 1.80 | 1.80 |
| | Stearyl alcohol | 2.50 | 2.50 | 2.50 | 2.50 | 2.50 |
| | Dimethyl Capramide | 5.00 | 5.00 | 5.00 | 5.00 | 5.00 |
| | Ethylhexyl Triazone | | 3.00 | | | |
| | Octocrylene | 6.00 | 6.00 | 6.00 | 6.00 | 6.00 |
| | Butyl Methoxydibenzoyl- | 2.00 | 2.00 | 2.00 | 2.00 | 2.00 |
| | methane | | | | | |
| | Titanium Dioxide and Aluminum | | | | 3.80 | |
| | Hydroxide and | | | | | |
| | Stearic Acid | | | | | |
| | Ethylhexyl Methoxycinnnamate | | | | | 3.00 |
| | Bis-Ethylhexyloxyphenol Methoxyphenyl | 1.00 | 1.00 | 1.00 | 1.00 | 1.00 |
| | Triazine (Tinosorb | | | | | |
| | S) | | | | | |
| Part B | Aqua | Qs to 100 | Qs to 100 | Qs to 100 | Qs to 100 | Qs to 100 |
| | Disodium EDTA | 0.20 | 0.20 | 0.20 | 0.20 | 0.20 |
| | Xanthane gum | 0.30 | 0.30 | 0.30 | 0.30 | 0.30 |
| | Potassium Cetyl Phosphate | 2.50 | 2.50 | 2.50 | 2.50 | 2.50 |
| Part C | Aqua | | | 5.00 | | |
| | Phenylbenzimidazole Sulfonic | | | 3.00 | | |
| | Acid | | | | | |
| | Tromethamine | | | 1.35 | | |
| Part D | Methylene Bis-Benzotriazolyl | 4.50 | 4.50 | 4.50 | 4.50 | 4.50 |
| | Tetramethylbutylphenol | | | | | |
| | (and) Aqua (and) Decyl | | | | | |
| | Glucoside (and) Propylene | | | | | |
| | Glycol (and) Xanthan Gum | | | | | |
| Part E | Sodium Acrylates Copolymer | 0.50 | 0.50 | | | 0.50 |
| | and Mineral Oil and | | | | | |
| | PPG-1 Trideceth-6 | | | | | |
| Part F | Phenoxyethanol (and) Methylparaben | 1.00 | 1.00 | 1.00 | 1.00 | 1.00 |
| | (and) Ethylparaben | | | | | |
| | (and) Butylparaben | | | | | |
| | (and) Propylparaben (and) | | | | | |
| | Isobutylparaben | | | | | |
| Part G | NaOH 30% | | | 11 drops | | |
| | In Vitro SPF | 20.7 | 38.8 | 26.4 | 32.6 | 28.8 |
| | UVA/UVB ratio | 0.85**** | 0.67*** | 0.75*** | 0.78*** | 0.77*** |

1. UV filter composition comprising
(a) an organic UV absorber of formula (1) 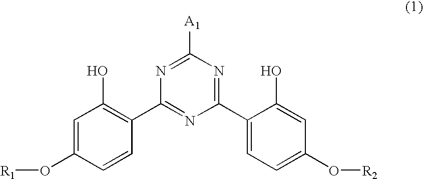
wherein
R₁ and R₂, independently of one another, are C₃-C₁₈alkyl; C₂-C₁₈alkenyl; a radical of the formula —CH₂—CH(OH)—CH₂—O-T₁; or
R₁ and R₂ are a radical of the formula (4a) 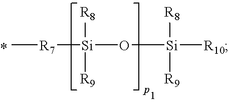
R₇ is a direct bond; a straight-chain or branched C₁-C₄alkylene radical or a radical of the formula 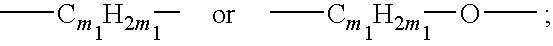
R₈, R₉ and R₁₀, independently of one another, are C₁-C₁₈alkyl; C₁-C₁₈alkoxy or a radical of the formula 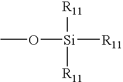
R₁₁ is C₁-C₅alkyl;
m₁ is a number from 1 to 4;
p₁ is 0; or a number from 1 to 5;
A₁ is a radical of the formula (1b), (1c) or (1d) 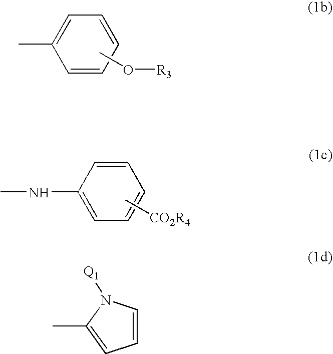
R₃ is hydrogen; C₁-C₁₀alkyl; —(CH₂CHR₅—O)ₙ_{₁}T₂; or —CH₂—CH(—OH)—CH₂—O-T₂;
R₄ is hydrogen; M; C₁-C₅alkyl; or —(CH₂)ₘ_{₂}—O-T₃;
R₅ is hydrogen; or methyl;
T₁, T₂ and T₃ independently from each other are hydrogen; or C₁-C₈alkyl;
Q₁ is C₁-C₁₈alkyl;
M is a metal cation;
m₂ is 1 to 4; and
n₁ is 1-16;
(b) dimethyl capramide;
(c) a cosmetically acceptable carrier; and
water.

2. Composition according to claim 1, wherein in formula (1) A₁ is a radical of the formula (1a₁), (1a₂), (1b₁) or (1b₂) 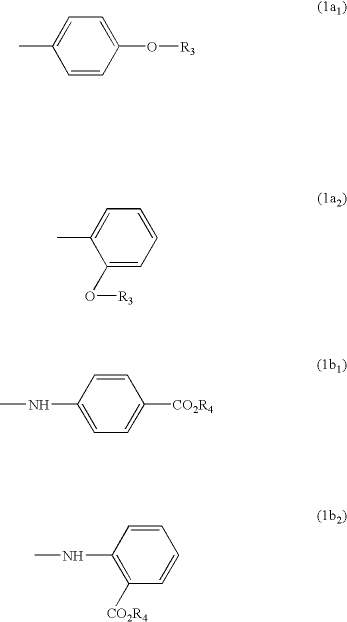
wherein
R₃ and R₄ are as defined in claim 1.

3. A composition according to claim 1, wherein
(a) corresponds to the formula (2a), (2b), (3a) or (3b) 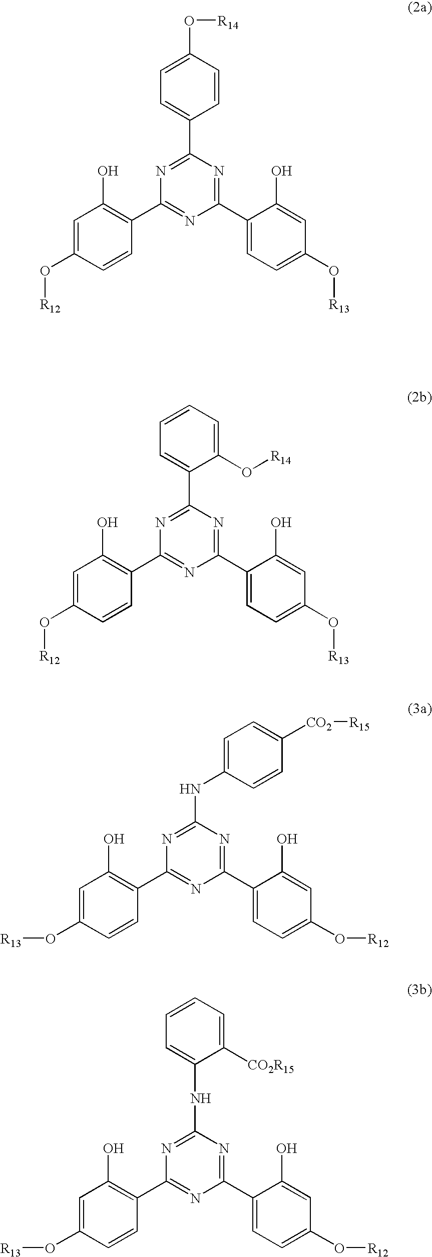
in which
R₁₂ and R₁₃, independently of one another, are C₃-C₁₈alkyl; or —CH₂—CH(—OH)—CH₂—O-T₁;
R₁₄ is C₁-C₁₀alkyl; or a radical of the formula 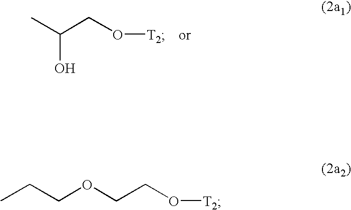
R₁₅ is hydrogen; M; C₁-C₅alkyl; or a radical of the formula —(CH₂)ₘ—O-T₂;
M is an alkali metal cation;
T₁ and T₂, independently of one another, are hydrogen; or C₁-C₅alkyl; and
m is 1 to 4.

4. Composition according to claim 3, wherein in the formulae (2a) and (2b),
R₁₂ and R₁₃, independently of one another, are C₃-C₁₈alkyl or —CH₂—CH(—OH)—CH₂—O-T₁;
R₁₄ is C₁-C₁₀alkyl; and
T₁ is hydrogen; or C₁-C₅alkyl.

5. Composition according to claim 3, wherein the formulae (3a) and (3b),
R₁₂ and R₁₃, independently of one another, are C₃-C₁₈alkyl or —CH₂—CH(—OH)—CH₂—O-T₁;
R₁₅ is hydrogen; M; or C₁-C₅alkyl; and
T₁ is hydrogen; or C₁-C₅alkyl.

6. Composition according to claim 3, wherein the formula (2a), (2b), (3a) or (3b), R₁₂ and R₁₃ have the same meaning.

7. Composition according to claim 1, wherein the organic UV absorber (a) is 2,4-Bis-{[4-(2-ethyl-hexyloxy)-2-hydroxy]-phenyl}-6-(4-methoxyphenyl)-1,3,5-triazine.

8. Composition according to claim 1, wherein one or more than one additional UV absorber (d) is used.

9. Composition according to claim 1, which comprises
20 to 70% of component (a),
25 to 75% b.w. of component (b), and
5 to 20% b.w. of component (c),
based on the total weight of the UV absorber composition.

10. Composition according to claim 1, which comprises an additional lipophilic solubilizing agent selected from benzoate esters or derivatives, glyceryl esters or derivatives, alkoxylated alcohol esters or ethers, fats, oils, fatty alcohols, hydrocarbons and ethers.

11. Cosmetic or pharmaceutical preparation comprising 0.05% to 80%, based on the total weight of the composition, of the UV absorber composition according to claim 1.

12. (canceled)

13. (canceled)
